(19) United States
(12) Patent Application Publication
(10) Pub. No.: US 2025/0170183 A1
(43) Pub. Date: May 29, 2025

(54) METHOD FOR ENRICHING MUSE CELLS AND OBTAINING EXOSOMES, MICROVESICLES OR THE SECRETOME THEREFROM

(71) Applicant: JLM ExoGrade, LLC, Austin, TX (US)

(72) Inventors: Michael COLGAN, Austin, TX (US); Adas DARINSKAS, Kaunas (LT)

(21) Appl. No.: 18/841,035

(22) PCT Filed: Feb. 27, 2023
(86) PCT No.: PCT/US2023/013996

§ 371 Date: Aug. 23, 2024

Related U.S. Application Data
(60) Provisional application No. 63/268,645, filed on Feb. 28, 2022.

(51) Int. Cl. A61K 35/28; A61P 29/00; C12N 5/0775

(52) U.S. Cl. CPC A61K 35/28; A61P 29/00; C12N 5/0668; C12N 2500/25; C12N 2500/38; C12N 2501/113; C12N 2501/125; C12N 2501/13; C12N 2501/165

(57) ABSTRACT

The invention relates to a method of enriching Muse cells and obtaining the exosomes, microvesicles, or the secretome therefrom, in several embodiments, the exosomes, microvesicles, secretome or Muse cells may be used in therapeutic methods or non-therapeutic methods.

### FIELD OF THE INVENTION

The present application relates generally to methods of enriching Muse cells in vitro, and producing extracellular vesicles (EVs) from such Muse cell-enriched cultures. The application further relates to using the Muse cell-derived EVs or fractions thereof, such as exosomes or microvesicles, or compositions comprising the same, in therapeutic methods in order to repair and/or regenerate damage or diseased tissues. The application further provides the EVs or fractions thereof, such as exosomes or microvesicles, or compositions comprising the same, for use in non-therapeutic methods.

### BACKGROUND

Mesenchymal stem cells (MSCs) are easily accessible from mesenchymal tissues, such as bone marrow and adipose tissue, and consist of a heterogeneous population. Recent studies suggest that a small subpopulation called multilineage-differentiating stress-enduring (Muse) cells might represent the cells that confer pluripotent-like properties to MSCs. Muse cells can form cells representative of all three germ layers, and can self-renew, but in contrast to MSCs are non-tumorigenic.

Muse cells are further attractive for therapeutic applications, because they can efficiently migrate and integrate into damaged tissue when administered and spontaneously differentiate into cells compatible with the homing tissue.

Exosomes and microvesicles are small membranous vesicles secreted by most cell types, including stem cells, such as mesenchymal stem cells (MSCs). Recent studies suggest that the secretome, i.e. the entirety of secreted agents including vesicles, peptides, cytokines, growth factors, proteins, lipids, metabolites, and nucleic acids, might confer regenerative capacity to MSCs, which is an active area of studies. However, not much is known about the exosomes and microvesicles, or the secretome, of Muse cells, which is partly due to the difficulty of obtaining Muse cells in suitable quantities.

The present invention solves this problem by providing a method of highly enriching Muse cells. The enrichment methods thus allows for the first time to obtain the secretome, the exosome or the microvesicles from Muse cells or Muse cell-enriched cultures.

### SUMMARY OF THE INVENTION

The inventors have surprisingly demonstrated that it is possible to enrich Muse cells to a high degree by using the provided method, which enables the isolation of extracellular vesicles (EVs) from Muse cells or cultures enriched in Muse cells, which are used interchangeably herein. In particular, the Muse cell-derived EVs have immunomodulatory and regenerative properties that make them useful for the treatment of including, but not limited to inflammatory disorders, and disorders characterized by neuro-inflammation, including Covid-19, traumatic brain injury (TBI) and chronic traumatic encephalopathy (CTE) and migraine.

In the following, the first aspect which pertains to plurality of extracellular vesicles (EVs) obtained from Muse cells or a tissue comprising Muse cells; the second aspect which pertains to a composition comprising EVs; the third aspect, which pertains to a method for preparing EVs from Muse cells; the fourth aspect which pertains to EVs prepared from said methods; the fifth aspect which pertains to a pharmaceutical composition; the sixth aspect which pertains to the pharmaceutical compositions for use in various therapies; the seventh aspect which pertains to non-therapeutic uses of the pharmaceutical compositions; the eighth aspect which pertains to a dosage form; the ninth aspect which pertains to use of the pharmaceutical composition for the manufacture of a medicament, will be described in further detail.

It shall be understood that topics which are discussed for, e.g., the first aspect will also apply for the second aspect, and any other aspects unless the technical context requires otherwise or unless the discussed topic clearly pertains to one aspect only. For example, in the context of the first aspect it is mentioned that extracellular vesicles (EVs) are obtained from Muse cells or a tissue comprising Muse cells. If in the context of the second aspect or another aspect, the EVs are mentioned, this also means that the same EVs of the first aspect are used, even if this was not explicitly mentioned for the second or further aspect.

The disclosure for the first, second, and any other aspect shall thus be assessed in combination as reflecting embodiments of the same overarching principles, and not as unrelated, separate chapters.

In a first aspect of the invention relates to plurality of extracellular vesicles (EVs) obtained from Muse cells or a tissue comprising Muse cells. The EVs comprise the entirety of secreted vesicles from Muse cells.

The Muse cells or tissue comprising Muse cells can be cultured cells which are enriched for Muse cells, e.g. comprise Muse cells in a concentration that is enriched by a factor of 2, 3, 4, 5, 10, 100, 10³, 10⁴, 10⁵ or more relative to the amount of Muse cells present prior to enrichment.

In some embodiments, the plurality of EVs, such as EV100 or EV500 or a combination thereof, is present in a concentration of about 10³-10¹¹, 10³-10¹⁰, 10⁴-10⁹, 10⁵-10¹¹ per/ml. In a particular embodiment, the plurality of EVs, such as EV100 or EV500 or a combination thereof, is present in a concentration of about 10%-10¹¹ per/ml, optionally about 10⁷-10¹¹ EVs per/ml.

In some embodiments, the EVs have a mean diameter of about 1000 nm or less, preferably, wherein the mean diameter is 550 nm or less.

In some embodiments, the plurality of EVs is isolated. In some embodiments, the EVs are selected from a group comprising exosomes (EV100) and/or microvesicles (EV500). The EVs can be substantially free of other agents, i.e. are purified. The EVs can be provided comprised in a composition comprising other agents and soluble factors secreted by the Muse cells or cell culture enriched for Muse cells. The other agent or soluble factors secreted by the Muse cells or cell culture enriched for Muse cell can be the secretome of said Muse cells or culture.

In a second aspect, the invention provides a composition comprising the plurality of EVs obtained from Muse cells or a tissue comprising Muse cells. The composition can further comprise EVs from mesenchymal stem cells (MSCs).

In some embodiments, the composition further comprises soluble factors, such as proteins, cytokines, secreted by the Muse cells or Muse cell enriched culture. In some embodiments, the composition comprises the secretome of Muse cells or Muse cell enriched culture. In some embodiments, the composition of the invention according to the second aspect comprises soluble proteins of the secretome. In some embodiments, the composition of the invention according to the second aspect comprises nucleic acids, such as miRNAs, or IncRNA that are comprised in the secretome of Muse cells. In some embodiments, the composition of the invention according to the second aspect comprises cytokines, hormone-like substances and/or immunomodulatory substances.

In a third aspect, the invention provides a method for preparing EVs from Muse cells comprising the steps of:
(a) providing Muse cells;
(b) culturing the Muse cells of (a) in a first medium for a time period sufficient to expand Muse cells in an undifferentiated state;
(c) culturing the Muse cells of (c) in a second medium for a time period sufficient to induce production of EVs, wherein the EVs are released into the second medium; and
(d) harvesting the EVs for one or more times, wherein each harvesting comprises collecting the second medium comprising the EVs to obtain a harvest.

The inventive method for preparing EVs is based on enriching the Muse cell population in a MSC culture, and obtaining EVs from said Muse cell enriched culture. In some embodiments, the Muse cells are enriched by a factor of at least 2, 5, 10, 20, 30, 40, 50 or 60. In some embodiments, the Muse cell-derived EVs are repeatedly harvested to further increase the yield of the obtained EVs. Embodiments discussed in the context of the methods and/or compositions of the invention may be employed with respect to any other method or composition described herein. Thus, an embodiment pertaining to one method or composition may be applied to other methods and compositions of the invention as well.

In some embodiments, the Muse cells are obtained from mesenchymal stem cells, adipose-derived stem cells, bone-marrow-derived stem cells, placenta, umbilical cord, cord blood, Wharton's jelly, cartilage, adipose tissue, bone marrow or dermal fibroblastic tissue.

In a fourth aspect, the invention provides a plurality of EVs obtainable by the inventive method.

The method of the invention provides an efficient method of highly enriching a MSC culture for Muse cells. The plurality of EVs obtained therefrom will be consequently also enriched from Muse cell-derived EVs.

In a fifth aspect, the invention provides a pharmaceutical composition comprising (i) a pharmaceutically effective amount of a plurality of EVs or the composition according to the invention, and a pharmaceutically acceptable carrier.

In some embodiments, the pharmaceutical composition further comprises an immunomodulatory agent, anti-inflammatory agent or therapeutic agent. In some embodiments, the immunomodulatory agent, anti-inflammatory agent or therapeutic agent is comprised in the EVs. In some embodiments, the immunomodulatory agent, anti-inflammatory agent or therapeutic agent comprises a cytokine, protein, growth factor, lipid, metabolite miRNA or IncRNA.

In some embodiment, the immunomodulatory agent, anti-inflammatory agent or therapeutic agent is conjugated to the EVs. In some embodiments, the conjugated immunomodulatory agent, anti-inflammatory agent or therapeutic agent comprises an antibody, cytotoxic agent, chemotherapeutic agent or other drug.

In some embodiments, the pharmaceutical composition is formulated for administration via the rectal, intranasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir into the subject.

The pharmaceutical composition can be administered topically or systemically.

In a sixth aspect, the invention provides the pharmaceutical composition of the invention for use in therapy.

In some embodiments, the invention provides the pharmaceutical composition of the invention for use in a method of treating cancer or a tumor in a subject.

In some embodiments, the tumor or cancer is cancer of the breast, liver, pancreas, lymph nodes, lung, skin, bone marrow, adipose tissue, nerve tissue, glioma, stomach, colon, blood, kidney, or endometrium. cancer of the breast, liver, pancreas, lymph nodes, lung, skin, bone marrow, adipose tissue, nerve tissue, glioma, stomach, colon, blood, kidney, or endometrium.

The pharmaceutical composition for the therapeutic use comprises a pharmaceutically effective amount of a plurality of EVs, wherein the EVs may comprise exosomes (EV100), microvesicles (EV500) or both. In some embodiments, the pharmaceutical composition for the therapeutic uses comprises a pharmaceutically effective amount of EV100. In some embodiments, the pharmaceutical composition for the therapeutic uses comprises a pharmaceutically effective amount of EV500.

In some embodiments, the pharmaceutical composition for the therapeutic uses further comprises soluble factors or the secretome secreted by Muse cells. In some embodiments, the secreted factors comprise secreted proteins, antibodies, and/or cytokines. In some embodiments, the secreted factors enhance the anti-tumorigenic effect of the pharmaceutical composition.

In some embodiments, the pharmaceutical composition is administered with at least one additional anti-cancer agent. In some embodiments, the anti-cancer agent is an alkylating agent, antimetabolite, hormone, miRNA, chemotherapeutic agent, or cytotoxic agent.

In a further embodiment, the invention provides a pharmaceutical composition for use in a method of treating inflammation or an inflammatory disorder in a subject in need thereof, said method comprising administering a therapeutically effective amount of said composition to said subject.

In some embodiments, the inflammation or an inflammatory disorder is a recurrent inflammation or an inflammatory disorder. In some embodiments, the inflammation or an inflammatory disorder is an acute inflammation or an inflammatory disorder. In some embodiments, the inflammation or an inflammatory disorder is a chronic inflammation or an inflammatory disorder.

In some embodiments, the inflammation or an inflammatory disorder comprises rheumatoid arthritis, osteoarthritis, diabetes mellitus, Type 1 diabetes mellitus, inflammatory bowel disease, asthma, endometriosis, cancer, alopecia, inflammatory arthritis, psoriatic arthritis, lupus, juvenile idiopathic arthritis, gout, systemic lupus erythematosus, and seronegative spondyloarthropathies, relapsing polychondritis neurological inflammation, multiple sclerosis, migraine, Traumatic Brain Injury (TBI) and Chronic Traumatic Encephalopathy (CTE). In a particular embodiment, the EVs, such as EV100 or EV500, or the pharmaceutical composition is used in a method of treating an inflammation of cartilage, such as osteoarthritis.

In one embodiment, the pharmaceutical composition is administered with at least one anti-inflammatory agent. In some embodiments, the anti-inflammatory agent comprises an anti-inflammatory peptide or protein, or an anti-inflammatory nucleic acid.

In a particular embodiment, the EVs, such as EV100 or EV500, or the pharmaceutical composition is used in a method of treating an inflammation of cartilage, such as osteoarthritis in combination with at least one anti-inflammatory agent. In some embodiments, the anti-inflammatory agent comprises an anti-inflammatory peptide or protein, or an anti-inflammatory nucleic acid.

In a particular embodiment, the EVs, such as EV100 or EV500, or the pharmaceutical composition is used in a method of treating a systemic inflammation in combination with at least one anti-inflammatory agent. In some embodiments, the anti-inflammatory agent comprises an anti-inflammatory peptide or protein, or an anti-inflammatory nucleic acid. The systemic inflammation comprises one or more of cardiovascular disease, cancer, diabetes mellitus, chronic kidney disease, non-alcoholic fatty liver disease, autoimmune and neurodegenerative disorders or coronary heart disease. In some embodiments, the systemic inflammation is associated with cancer. In a further embodiment, the systemic inflammation is associated with diabetes mellitus.

In some embodiments, the anti-inflammatory RNA comprises noncoding RNAs (ncRNAs), or microRNAs (miRNAs), optionally the miRNAs of Table 5 or 6. In some embodiments, the miRNAs comprise one or more of miR-199a-3p, miR-143-3p, miR-21-5p, miR-125b-5p, let-7b-5p, miR-29a-3p, let-7i-5p, miR-125a-5p, miR-16-5p, miR-221-3p, miR-432-5p, miR-127-3p, miR-382-5p, miR-146a-5p, miR-431-5p, miR-199b-3p, miR-100-5p, miR-6504-3p, miR-26a-5p, let-7a-5p, let-7f-5p, miR-26a-5p, miR-431-5p, miR-126-3p, miR-181a-5p, or let-7e-5p.

In another embodiment, the miRNAs comprise one or more of miR-199a-3p, miR-143-3p, miR-21-5p, miR-125b-5p, let-7b-5p, miR-29a-3p, let-7i-5p, miR-125a-5p, miR-16-5p, miR-221-3p, miR-432-5p, miR-127-3p, miR-382-5p, miR-146a-5p, miR-431-5p, miR-199b-3p, miR-100-5p, miR-6504-3p, miR-26a-5p, let-7a-5p, let-7f-5p, miR-126-3p, miR-181a-5p, let-7c-5p, or let-7g-5p.

In another embodiment, the miRNAs comprise one or more of miR-16-5p, miR-21-5p, miR-26a-5p, miR-29a-3p, miR-100-5p, miR-125a-5p, miR-125b-5p, miR-126-3p, miR-127-3p, miR-143-3p, miR-146a-5p, miR-181a-5p, miR-199a-3p, miR-199b-3p, miR-382-5p, miR-432-5p, miR-6504-3p, let-7a-5p, let-7b-5p, let-7c-5p, let-7f-5p, let-7g-5p, or let-7i-5p.

In a further embodiment, the invention provides a pharmaceutical composition for use in a method of restoring health in a subject in need thereof, said method comprising administering a therapeutically effective amount of said composition to said subject. In some embodiments, the subject suffers from stroke, myocardial infarction, age-related cognitive decline, or age-related loss of motor function. In further embodiments, the subject suffers from acute or chronic inflammation, such as inflammation of the cartilage.

In some embodiments, the subject is above the age of 50, 60, 70 or 80 years. In some embodiments, the administration of the EVs, such as EV100, EV500 or a combination thereof, or the pharmaceutical composition of the invention induces repair or regeneration of cells or tissues affected by a wide variety of tissue damage or disease. The EVs, such as EV100, EV500 or a combination thereof, or the pharmaceutical composition of the invention are for use in a method of treating inherited diseases, cellular or bodily dysfunctions, aberrant cellular ageing, and to modulate immune function.

In some embodiments, the administration of the pharmaceutical composition reverses aberrant cellular ageing. Cellular ageing can be determined by the presence of or status of one or more epigenetic marks.

In a seventh aspect, the invention provides non-therapeutic uses of the pharmaceutical compositions.

In one embodiment, the non-therapeutic use is directed to preventing hair loss or promoting hair growth in a subject. In some embodiments, the administration of the pharmaceutical composition of the invention inhibits or decreases hair loss. In some embodiments, hair loss affects the scalp or the full body. In some embodiments, the subject is human, wherein the human is female or male. In some embodiments, the hair loss is male pattern baldness. In some embodiments, the hair loss is female pattern baldness.

In some embodiments, the pharmaceutical composition is administered in conjunction with a further agent, wherein the further agent is optionally Minoxidil.

The non-therapeutic use can be directed to improving the appearance of wrinkles of the skin of a subject. The improvement of the appearance of wrinkles of the skin comprises a reduction of the number of wrinkles, a reduction of the density of wrinkles, or reduction of depth of wrinkles.

In an eight aspect, invention provides a dosage form comprising the pharmaceutical composition of the invention.

In some embodiments, the dosage form is selected from the group comprising tablet, capsule, gel-capsule, aerosol, liquid solution, eye-drop, film, cream, gel, film, or ointment.

In a ninth aspect, the invention provides a use of the pharmaceutical composition for the manufacture of a medicament.

### BRIEF DESCRIPTION OF THE FIGURES

FIG. 1 is a graphical overview of the production method of extracellular vesicles (EVs) from Muse cells, wherein the Muse cells are isolated, expanded and enriched resulting in a culture that comprises at least 10% and up to 80% Muse cells. The EVs from the Muse cell enriched cultures can be collected, i.e. harvested.

FIG. 2 is a graphical overview of the production method of extracellular vesicles (EVs) from Muse cells, showing that the method of the invention uses an adherent culture.

FIG. 3 shows FACS plots of a Muse cell enriched culture from umbilical cord. A: quantification for SSEA-3; B: quantification of CD105; C: quantification of CD73; D: quantification of CD90.

FIG. 4 shows the particle distribution of an EV100 sample using Nanosight.

FIG. 5 shows the immediate response of the anti-inflammatory effect of EV100 or Muse cells in the paw edema model of inflammation. The measurements were taken 1.5 hours after a single administration of the therapeutic agents. Saline was used as negative control. Dexamethasone was used as a positive control.

FIG. 6 shows the early response of the anti-inflammatory effect of EV100 or Muse cells in the paw edema model of inflammation 1 day post-administration of a single therapeutic agents. Saline was used as a negative control. Dexamethasone was used as a positive control.

FIG. 7 shows the intermediate response of the anti-inflammatory effect of EV100 or Muse cells in the paw edema model of inflammation 10 days post-administration of a single therapeutic agents. Saline was used as negative control. Dexamethasone was used as a positive control.

FIG. 8 shows the late response of the anti-inflammatory effect of EV100 or Muse cells in the paw edema model of inflammation 2 weeks post-administration of a single therapeutic agents. Saline was used as a negative control. Dexamethasone was used as a positive control.

FIG. 9 shows the anti-inflammatory effect of EV100 in the paw edema model of inflammation 1 day (24 hours) post-administration of the last dose. The therapeutic agents were administered on 5 consecutive days: day 0 (0.5 hours after the induction of inflammation by carrageenan), day 1, day 2, day 3 and day 4. Saline was used as negative control. Dexamethasone was used as a positive control.

FIG. 10 shows the anti-inflammatory effect of EV100 in the paw edema model of inflammation 6 days (146 hours) post-administration of the last dose. The therapeutic agents were administered on 5 consecutive days: day 0 (0.5 hours after the induction of inflammation by carrageenan), day 1, day 2, day 3 and day 4. Saline was used as negative control. Dexamethasone was used as a positive control.

FIG. 11 shows late the anti-inflammatory effect of EV100 in the paw edema model of inflammation 11 days (266 hours) post-administration of the last dose. The therapeutic agents were administered on 5 consecutive days: day 0 (0.5 hours after the induction of inflammation by carrageenan), day 1, day 2, day 3 and day 4. Saline was used as negative control. Dexamethasone was used as a positive control.

FIG. 12 shows FACS plots of a Muse cell enriched culture from human amniotic epithelial stem cells (hAESCs). The plots show the quantification for SSEA-3, CD90, CD105 and CD73 at passages 4, 6 and 8 of the same culture. The table provides the cell counts in percentages for each sample.

FIG. 13 shows serum levels of alanine aminotransferase (ALAT), aspartate aminotransferase (ASAT), and total protein before and 10 days after treatment of mice with vehicle control (VC), high Muse cell MSC low-dose (1×10⁹ cells/mouse), high Muse cell MSC medium-dose (5×10⁸ cells/mouse), high Muse cell MSC high-dose (10×10⁸ cells/mouse), high Muse cell MSC exosomes low-dose (10×10⁹ exosomes/mouse), high Muse cell MSC exosomes medium-dose (50×10⁹ exosomes/mouse), high Muse cell MSC exosomes high-dose (100×10⁹ exosomes/mouse). Mice in the VC group were administered PBS. High Muse cell MSC are labelled “MSC”, High Muse cell MSC exosomes are labelled “ExoMSC”.

FIG. 14 shows blood hematology results before and 10 days after treatment of mice with vehicle control (VC), high Muse cell MSC low-dose (1×10⁸ cells/mouse), high Muse cell MSC medium-dose (5×100 cells/mouse), high Muse cell MSC high-dose (10×10⁸ cells/mouse), high Muse cell MSC exosomes low-dose (10×10⁹ exosomes/mouse), high Muse cell MSC exosomes medium-dose (50×10⁹ exosomes/mouse), high Muse cell MSC exosomes high-dose (100×10⁹ exosomes/mouse). Mice in the VC group were administered PBS. High Muse cell MSC are labelled “MSC”, High Muse cell MSC exosomes are labelled “ExoMSC”. A: mean corpuscular volume (MCV), B: mean corpuscular hemoglobin (MCH), C: Neutrophils, D: Lymphocytes, E: Monocytes, F: Eosinophiles, G: Basophils, H: white blood cell count (WBC), I: Platelets, J: red blood cell count (RBC), K: plateletcrit (PCT), L: mean platelet volume (MVP), M: haematocrit (Hct), N: haemoglobin concentration (HGB), O: H&E staining of the histopathological sections of the lungs, kidneys, liver, heart and spleen in the control (PBS) and high-dose experimental groups (high Muse MSC and high Muse MSCExo). Scale bar 400× (50 μm).

FIG. 15 A shows the anti-inflammatory effect of high Muse cell MSC-derived exosomes on the Murine Sepsis Severity Score of the LPS-induced sepsis rodent model at 6 h and 24 h post-administration of LPS. The high Muse cell MSC-derived exosomes were administered 48 h and 24 h before LPS administration and 1 h after LPS administration. PBS was used as negative treatment control. Dexamethasone was used as a positive treatment control and was administered 1 h after LPS.

FIG. 15 B-L shows the effect of high Muse cell MSC-derived exosomes on blood biochemistry and hematology of the LPS-induced sepsis rodent model at 6 h post-administration of LPS. The high Muse cell MSC-derived exosomes were administered 48 h and 24 h before LPS administration and 1 h after LPS administration. PBS was used as negative treatment control. Dexamethasone was used as a positive treatment control and was administered 1 h after LPS. WBC=white blood cell count, ALAT=alanine aminotransferase, ASAT=aspartate aminotransferase.

FIG. 16 shows the anti-inflammatory effect of high Muse cell MSC-derived exosomes in plasma samples of the LPS-induced sepsis rodent model at 6 h and 24 h post-administration of LPS. The high Muse cell MSC-derived exosomes were administered 48 h and 24 h before LPS administration and 1 h after LPS administration. PBS was used as negative treatment control. Dexamethasone was used as a positive treatment control and was administered 1 h after LPS.

FIG. 17 shows the anti-inflammatory effect of high Muse cell MSC-derived exosomes in plasma (A) and lung (B) samples of the LPS-induced sepsis rodent model. Graph shows the relative change from 6 h to 24 h post LPS induction as percentage change at 24 h compared to 6 h. The high Muse cell MSC-derived exosomes were administered 48 h and 24 h before LPS administration and 1 h after LPS administration. PBS was used as negative treatment control. Dexamethasone was used as a positive treatment control and was administered 1 h after LPS.

### DETAILED DESCRIPTION AND PREFERRED EMBODIMENTS

### Definitions

Unless otherwise indicated, the terms listed below will be used and are intended to be defined as stated, unless otherwise indicated. Definitions for other terms can occur throughout the specification. It is intended that all singular terms also encompass the plural, active tense and past tense forms of a term, unless otherwise indicated.

As used herein, the term “Extracellular vesicles” (EV) refers to vesicles that are released from a variety of different cells, including cancer cells, stem cells, stem-like cells and Muse cells. Extracellular vesicles have a diameter of 30-550 nm and comprise exosomes (EV100) and microvesicles (EV500). Unless otherwise indicated, any disclosure for “EVs” is also intended to be individually disclosed for EV100 and EV500.

As described herein, two EV subpopulations, EV100 and EV500, have been identified. In certain embodiments, the EVs are EV100. In another embodiment, the EVs are EV500. The “EVs obtained from Muse cells or tissue comprising Muse cells” relates to EVs that are obtained in vitro from stem cell cultures that are enriched for Muse cells.

As used herein, the terms “exosomes” and “EV100” are used interchangeably. EV100 refers to a population of exosomes having a diameter of 30 to 220 nm. EV100 have regenerative and therapeutic capacity and do not trigger an immunologic response after administration. In some embodiments, exosomes have a diameter of about 30 nm to about 220 nm. In some embodiments, exosomes have a diameter of about 30 nm to about 100 nm. In some embodiments, exosomes have a diameter of about 30 nm to about 150 nm. In some embodiments, exosomes have a diameter of about 40 nm to about 150 nm. In some embodiments, exosomes have a diameter of about 40 nm to about 120 nm. EV100 can be identified by expression of one or more of CD9, CD63, CD81 or tetraspanins.

As used herein, the term “microvesicles” and “EV500” are used interchangeably. EV500 refers to a population of larger vesicles having a diameter of about >220 to 550 nm. EV500 have regenerative and therapeutic capacity and do not trigger an immunologic response after administration. In some embodiments, microvesides have a diameter of about 220 nm to about 550 nm. In some embodiments, microvesicles have a diameter of about 220 nm to about 300 nm.

The term “mesenchymal stem cell” or “MSC”, as used herein, refers to a multipotent somatic stem cell derived from mesoderm, having self-regenerating and differentiating capacity to produce progeny cells with a large phenotypic variety, including connective tissues, stroma of bone marrow, adipocytes, dermis and muscle, among others. MSCs generally have a cell marker expression profile characterized in that they are negative for the markers CD19, CD45, CD14 and HLA-DR, and positive for the markers CD105, CD106, CD90 and CD73. The MSCs can be derived from any animal, preferably a mammal including a non-primate (e g, a cow, pig, horse, cat, dog, rat, or mouse) and a primate (e g, a monkey, or a human). In a particular embodiment, the MSCs are derived from a human.

As understood in the art, the terms “tumor” and “cancer” are overlapping terms. A “tumor” is broadly considered to be a mass or growth found in an organism. A tumor cell is a cell derived from such a mass. A tumor can be benign or cancerous. A cancerous tumor, or “cancer” is a tissue growth that can spread out of control and invade other tissues, or in the case of blood cancers, overwhelm the circulatory system and/or seed cancers elsewhere in the body. A cancer cell is a cell derived from a cancer. For purposes of the invention, the terms “tumor cell” and “cancer cell”, or “tumor” and “cancer” are used interchangeably, with the understanding that both refer to mammalian cells found in tumors or cancers or derived from and cultured from tumors or cancers, and that replicate abnormally, without the limits exhibited by differentiated mammalian cells.

The term, “Muse cell” (multilineage-differentiating stress-enduring cell) is a pluripotent non-tumorigenic cell present in mesenchymal tissues. Muse cells are present in several tissues, including placenta, umbilical cord, cord blood, Wharton's jelly, cartilage, adipose tissue, bone marrow or dermal fibroblastic tissue, mesenchymal stem cells, adipose-derived stem cells, and bone-marrow-derived stem cells. In one embodiment, the Muse cells are obtained from umbilical cord, cord blood, placenta or Wharton's jelly.

Culturing Muse cells and expanding Muse cells in vitro is challenging, because Muse cells tend to differentiate in vitro and do not proliferate well in vitro.

Muse cells as used herein express stage-specific embryonic antigen 3 (SSEA-3) and the mesenchymal cell marker CD105. Muse cells may further express Nanog, Oct4, Klf4, c-Myc, and Sox2. Muse cells can differentiate in vitro into cells of the three germ layers, such as hepatocytes (endodermal), neural cells (ectodermal) and adipocytes, and osteocytes (mesodermal cells), i.e. they are pluripotent. Muse cells normally exist in a quiescent state, singularly activated by severe cellular stress in vitro and in vivo. Muse cells have the capacity for self-renewal while maintaining pluripotent cell characteristics indicated by the expression of pluripotent stem cell markers. Muse cells differentiate into cells representative of all three germ cell layers both spontaneously and under media-specific induction. In contrast to embryonic stem and induced pluripotent stem cells, Muse cells exhibit low telomerase activity, a normal karyotype, and do not undergo tumorigenesis once implanted in SCID mice.

While Muse cells share several mesenchymal stem cell markers with MSCs, such as CD105, CD73 and CD90, Muse cells in addition express SSEA3, which is not present in MSCs, and can be identified and/or isolated by SSEA3+ using FACS.

In contrast to MSCs, Muse cells have the capacity to home to damaged tissue to contribute to tissue regeneration and repair, and are thought to produce or carry along cytokines, trophic factors and anti-inflammatory factors.

However, until now, it has not been possible to culture Muse cells with high efficacy, which is required to produce and obtain Muse-cell derived extracellular vesicles, such as exosomes (EV100) and microvesicles (EV500).

There are major differences between Muse cells and non-Muse cells in present within mesenchymal cell population. When mesenchymal cells (sometimes called mesenchymal stem cells) are separated into Muse and non-Muse cells by SSEA-3 cell sorting, the following differences are observed:
(1) Muse cells, SSEA-3(+) form clusters (which are similar to embryoid bodies of ES cells) from a single cell in suspension, while non-Muse cells, SSEA-3(−) do not proliferate successfully in suspension and thus do not form these distinctive clusters.
(2) Basic expression level of pluripotency genes in non-Muse cells is very low or undetectable level compared to Muse cells.
(3) Non-Muse cells do not exhibit tissue reparation when infused into the blood stream.

While they do not integrate into the damaged tissue, they may indirectly contribute to tissue regeneration by their production of cytokines, trophic factors and anti-inflammatory factors.

In a preferred embodiment, Muse cells are obtained from umbilical cord, cord blood, placenta or Wharton's jelly. While it is feasible to enrich Muse cells from all tissues according to the method of the invention, the efficiency and yield is greatly increased if Muse cells are obtained from one or more of the preferred tissues, i.e. umbilical cord, cord blood, placenta and Wharton's jelly.

Muse cell can be derived from any animal, preferably a mammal including a non-primate (e g, a cow, pig, horse, cat, dog, rat, or mouse) and a primate (e g, a monkey, or a human). In a particular embodiment, the Muse cell or EVs obtained therefrom, are from a human.

Muse cells typically make up about 0.1-2% of cells in a tissue. However, after enrichment of Muse cells in culture using the method of the invention, Muse cells can be enriched from 2-fold to 10⁵-fold or more. Cultures of enriched Muse cells, for example umbilical cord-derived MSC, that have undergone the method of enriching Muse cells of the invention, are referred to as “high Muse cell” cultures, e.g. high Muse cell MSC cultures. In some embodiments, Muse cells are enriched 2, 5, 10, 100, 10³, 10⁴, 10⁵-fold relative to the concentration of Muse cells in the tissue the Muse cells were obtained from. In some embodiments, the Muse cells are enriched to be present in the high Muse cell culture after enrichment in a concentration of at least 10%, 15%, 20%, 30%, 35%, 40%, 45%, 50%, 55%, 60%, 65%, 70%, 80% or more.

In accordance with all aspects of the present invention, a “subject” or “patient” encompasses any animal, but preferably a mammal, e.g., human, non-human primate, a dog, a cat, a horse, a cow, or a rodent. More preferably, the subject or patient is a human. In some embodiments of the present invention, the subject has an inflammatory disorder, for example and without limitation, cancer, rheumatoid arthritis, osteoarthritis, a neuro-inflammatory disorder (such as Covid-19 or long Covid-19).

In some embodiments, the subject is treated by being administered a therapeutically effective dose of EVs, EV100, EV500 or Muse cells.

In some embodiments of the present invention, the subject has cancer, for example and without limitation, melanoma, breast cancer, or pancreatic cancer. In some embodiments, the cancer is a primary tumor, while in other embodiments, the cancer is a secondary or metastatic tumor.

The term “inflammatory conditions” refers to conditions that promote inflammation. For tissues in vivo, pro-inflammatory conditions include conditions that promote elevated levels of pro-inflammatory cytokines. For example, proinflammatory cytokines include, interferon-gamma, interleukin-la, interleukin-1-β, interleukin-6, tumor necrosis factor-a, and combinations thereof. The terms “inflammatory disorder” or “inflammation” are used interchangeably with “inflammatory conditions”.

The term “organ” or “tissue” refers to any solid or fluid organ of a subject, such as lung, pancreas, stomach, cartilage, intestine, bladder, kidney, ovary, testis, skin, colorectal, breast, prostate, brain, esophagus, liver, adipocyte, placenta, umbilical cord, Wharton's jelly, blood, plasma, serum, cerebrospinal fluid, amniotic fluid, and umbilical cord blood.

The terms “Exosomes” and “EV100” are used interchangeably and refer to small vesicles that are released from a variety of different cells, including Muse cells (i.e., “Muse cell-derived exosomes”). These small vesicles have a diameter of about 30-220 nm, and are derived from large multi vesicular endosomes and are secreted into the extracellular milieu. The precise mechanisms of exosome release/shedding remain unclear; however, this release is an energy-requiring phenomenon, modulated by extracellular signals. They appear to form by invagination and budding from the limiting membrane of late endosomes, resulting in vesicles that contain cytosol and that expose the extracellular domain of membrane-bound cellular proteins on their surface. Using electron microscopy, studies have shown fusion profiles of multivesicular endosomes with the plasma membrane, leading to the secretion of the internal vesicles into the extracellular environment. The rate of exosome release is significantly increased in most neoplastic cells and occurs continuously. Increased release of exosomes and their accumulation appear to be important in the malignant transformation process.

The term “culturing” refers to the in vitro maintenance, differentiation, and/or propagation of cells, preferably cells comprising Muse cells, in suitable media. The term “enriched” culture, refers to a composition or culture comprising components, e.g., Muse cells, present in a greater percentage of total cells than is found in the tissues where they are present in an organism or at the time prior to the enrichment process. As an example, a Muse cell-enriched culture obtained from umbilical cord tissue comprises a greater percentage of Muse cells than is found in umbilical cord tissue in an organism, i.e. in native umbilical cord.

The phrase “consisting essentially of” means that the composition or method may include additional ingredients and/or steps, but only if the additional ingredients and/or steps do not materially alter the basic and novel characteristics of the claimed composition or method, i.e., the additional ingredient and/or step(s) would serve no purpose material to the claimed composition or method.

An “effective amount” is an amount sufficient to effect beneficial or desired results. In an exemplary embodiment, the effective amount is the amount that results in the reduction of the levels of inflammatory cytokines or ameliorates the severity of symptoms associated with a disease or disorder. In a further exemplary embodiment, the effective amount brings about a reduced rate of progression of a tumor or cancer. An effective amount can be administered in one or more administrations, applications or dosages. The effective amount, i.e., a suitable dosage, will vary depending on body weight, age, health, disease or condition to be treated and route of administration. The dose of exosomes administered to a subject is in an amount effective to achieve the desired beneficial therapeutic response in the subject over time.

The person skilled in the art will be readily able to determine the amount of inactivated exosomes to be administered by titrating the dose and duration of administration to reach an optimal clinical response, such as a reduction in the severity or spread of an inflammation, the rate or degree of repair and/or regeneration, or the rate of progression and/or spread of a cancer.

### PREFERRED EMBODIMENTS

### Extracellular Vesicles and Compositions Comprising the Same

In a first aspect, the invention relates to a plurality of extracellular vesicles (EVs) obtained from Muse cells or a tissue comprising Muse cells. In a second aspect, the plurality of EVs are comprised in a composition.

In some embodiments, the Muse cells are cultured Muse cells. It is understood that Muse cells and Muse cell enriched culture are used interchangeably throughout the application.

In some embodiments, the plurality of EVs have a mean diameter of less than 1000 nm, 900 nm, 800 nm, 700 nm, 600 nm, 500 nm, 400 nm, 300 nm or 200 nm. Some embodiments, the EVs have a mean diameter of 30-1000 nm, 30-700 nm, or 30-550 nm.

In some embodiments, the plurality of EVs have a diameter of less than 550 nm, preferably wherein the EVs are obtained from Muse cells of the placenta, umbilical cord, cord blood or Wharton's jelly.

In some embodiments, the plurality of EVs have a diameter of 30-220 nm and are obtained from Muse cells of the placenta, umbilical cord, cord blood or Wharton's jelly. In some embodiments, the EVs have a diameter of 220-550 nm and are obtained from Muse cells of the placenta, umbilical cord, cord blood or Wharton's jelly.

In some embodiments, the plurality of EVs have a diameter of 30-220 nm and are obtained from Muse cells of the placenta, umbilical cord, cord blood or Wharton's jelly. In some embodiments, the EVs have a diameter of 220-550 nm and are obtained from Muse cells of the placenta, umbilical cord, cord blood or Wharton's jelly.

The particle size distribution can be determined by routine methods of the art. In some embodiments, the distribution of the EVs in a preparation or composition can be shown by D90 and/or D10 values.

In some embodiments, the D90 value of the plurality of EVs is 500 nm or less than 500 nm. In some embodiments, the D90 value is about 320-420 nm, preferably about 330 nm. In some embodiments, the D10 value is about 200 nm. In some embodiment, the D90 value is about 330 nm and the D10 value is about 220 nm.

In another embodiment, the D90 value of the plurality of EVs is 150 to 200, 160-180, preferably about 160-170 nm. In some embodiments, the D10 value is about 40-100 nm, 40-80 nm, preferably about 40-60 nm. In some embodiment, the D90 value is about 165 nm and the D10 value is about 45 nm.

The plurality of EVs, such as EV100 or EV500 or a combination thereof, can be substantially free of any excipients or additives. In some embodiments, the plurality of EVs according to the invention, wherein substantially all EVs are EV100 or EV500.

In some embodiments, the composition comprising the EVs of the first aspect further comprises EVs of MSCs. In some embodiments, the composition comprises further soluble factors, such as proteins, peptides, exomeres, growth factors, lipids, metabolites, or cytokines. In some embodiments, the composition comprises the secretome of the Muse cells or Muse cell enriched culture.

Tissue for obtaining Muse cells Muse cells can be enriched from any tissue type that comprises Muse cells, such as mesenchymal stem cells, adipose-derived stem cells, bone-marrow-derived stem cells, placenta, umbilical cord, cord blood, Wharton's jelly, cartilage, adipose tissue, bone marrow or dermal fibroblastic tissue. The naturally occurring relative amount of Muse cells in these tissues is typically between 0.1-2%, or 0.1-1%, which depends on the tissue type and age of the subject.

In some embodiments, the Muse cells are enriched from are obtained from umbilical cord, cord blood, placenta or Wharton's jelly. It is believed that these perinatal cell types retain particularly high therapeutic and regenerative capacity, rendering those preferred tissues or enriching Muse cells and obtaining EVs.

### Method of Enriching Muse Cells and Preparing EVs

In a third aspect, the invention relates to a method for preparing EVs derived from Muse cells, said method comprising
(a) providing Muse cells;
(b) culturing the Muse cells of (a) in a first medium for a time period sufficient to expand Muse cells in an undifferentiated state;
(c) culturing the Muse cells of (c) in a second medium for a time period sufficient to induce production of EVs, wherein the EVs are released into the second medium; and
(d) harvesting the EVs for one or more times, wherein each harvesting comprises collecting the second medium comprising the EVs to obtain a harvest.

The first medium is suitable to expand the Muse cells in an undifferentiated state. It is believed that Muse cells preferentially grow in the first medium, wherein the MSCs which are also present in the culture grow slower, thereby advantageously enriching for Muse cells.

In one embodiment, the first medium comprises growth factors and suitable nutrients to promote growth and proliferation of Muse cells. In some embodiments, the first medium comprises a mammalian cell adjusted basal medium (such as DMEM, RPMI or other), human cord blood plasma, human platelet lysate, fibroblast growth factor, stem cell factor, VEGF, transferrin, selenium, insulin, human growth hormone, nerve growth factor, vitamin C, D, E, A, and valproic acid.

In a particular embodiment, the first medium comprises DMEM 1-5 g/L glucose, 1-10% human cord blood plasma, 10-100 IU/ml heparin, 2-5% human PRP lysate, fibroblast growth factor 10-50 ng/L, stem cell factor 5-20 ng/L, VEGF 2-20 ng/L, transferrin 10-100 ng/L, selenium 50-200 ng/L, insulin 10-200 ng/L, human growth hormone 0.2-1 IU/L, nerve growth factor 50-200 ng/L, vitamin C, D, E, A, 0.2-5% and valproic acid 0.2-4%.

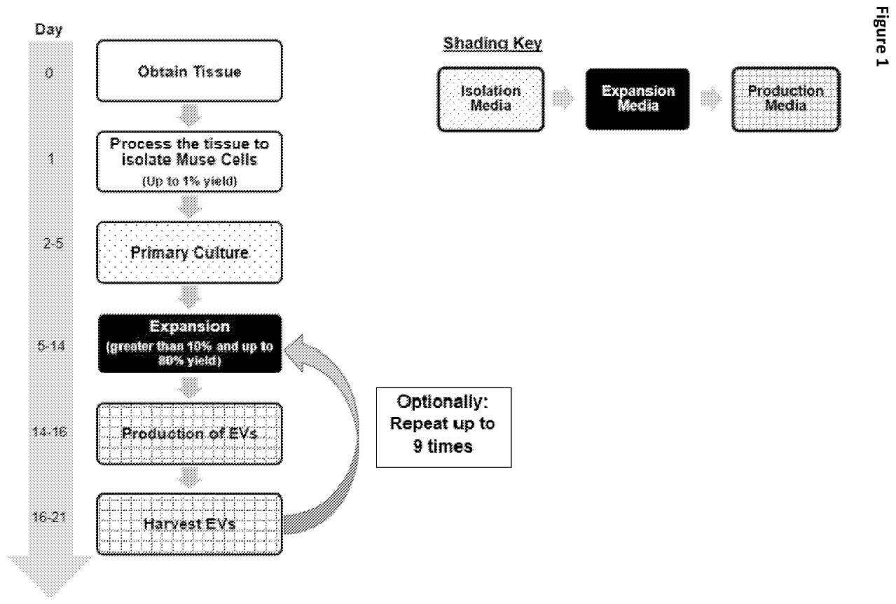

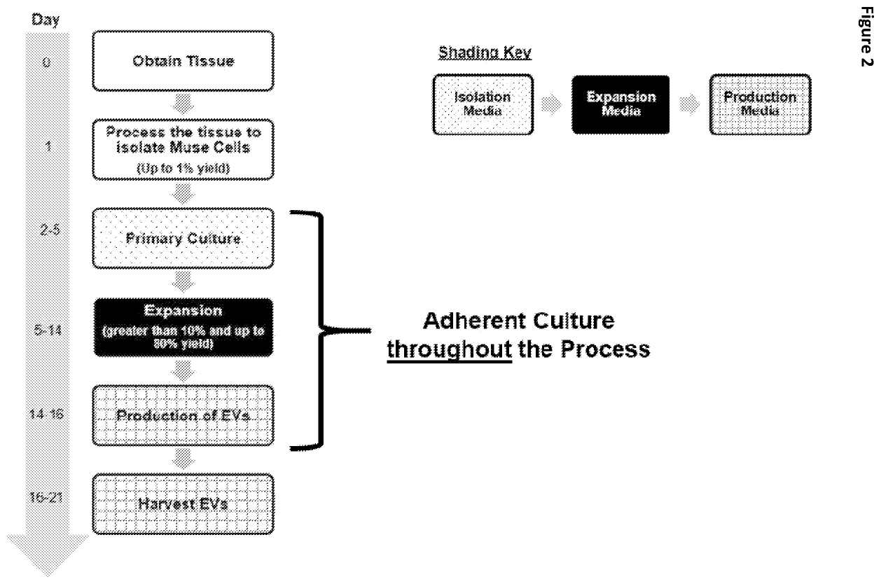

The first medium in the context of the inventive method is also called M2 (FIGS. 1 and 2). Prior to the expansion of Muse cells in the first medium, the tissue is minced and a single cell suspension is produced. The single cell suspension is plated in a plating medium (M1 in FIGS. 1 and 2) which supports the initial seeding and proliferation. The seeding medium, e.g. M1 medium comprises mammalian cell adjusted basal media (such as DMEM, RPMI or other), which has been supplemented with fetal calf serum, fetal bovine serum, human platelet lysate, insulin, or transferrin at a concentration of 1-25%.

In a particular embodiment, an exemplary M1 medium comprises DMEM with 1 g/L glucose, sodium pyruvate 110 mg/L, stable glutamine 200 mM±50, human platelet lysate (cryoprecipitate) 1-7.5%, fetal calf serum 1-10%, insulin 10-100 ng/L, transferrin 10-200 ng/L.

Alternatively, M1 medium consists of DMEM supplemented with human cord blood plasma, human platelet lysate, insulin, or transferrin at a concentration of 1-25%.

In one embodiment, M1 medium comprises DMEM with 1 g/L glucose, sodium pyruvate 110 mg/L, stable glutamine 200 mM±50, human platelet lysate (cryoprecipitate) 1-7.5%, human cord blood plasma 1-10%, insulin 10-100 ng/L, transferrin 10-200 ng/L.

In one embodiment, M1 medium comprises DMEM with 1 g/L glucose, sodium pyruvate 110 mg/L, stable glutamine 200 mM±50, human platelet lysate (cryoprecipitate) 5%, human cord blood plasma 5%, insulin 10-100 ng/L, transferrin 10-200 ng/L.

In some embodiments, the Muse cells are expanded and enriched during the proliferation phase by a factor of at least 2, 5, 10, 20, 30, 40, 50 or 60. For example, Muse cells which had an initial concentration of 0.1% can be enriched by said method to a concentration of about 40%, which corresponds to a 400-fold enrichment.

The enrichment and relative amount of Muse cells in the cell culture population can be determined by assaying for SSEA-3, which is specific for Muse cells and not expressed in the MSCs that are present in the culture, and support the undifferentiated state and growth of Muse cells.

After the Muse cells have been enriched and expanded, the cells, which are now highly enriched for Muse cells, can be induced to secrete EVs by switching the first medium, i.e. proliferation medium (e.g. M2) to a second medium, i.e. the EV production medium. The EV production medium does not support high proliferation, but in contrast induces the production of EVs into the cell culture medium.

The second medium that provides for the increased secretion of extracellular vesicles (EVs), including exosomes and microvesicles, comprises M3 phenol free media, DMEM or RMPI with a maximum of 5% of cord blood plasma or human platelet lysate.

The harvesting of (d) comprises replacing the medium comprising the EVs with a fresh second medium. The harvest comprises the secretome of the Muse cells. The Muse-cell enriched cell culture will then again secrete EVs into the freshly added second medium, e.g. M3, which may be harvested again. This step of adding new medium and harvesting the EVs, can be repeated once, twice, three times or more.

In some embodiments, the EV concentration is reduced with each subsequent harvest. To increase the output of EVs and maintain the undifferentiated state, the Muse cells can be re-plated (passaged) at sub-confluent density, repeating a cycle of expansion in the first medium, i.e. an expansion medium (e.g. M2 medium), followed by a switch to the second medium, i.e. the EV production medium (e.g. M3 medium). This harvest can again be repeated several times.

In some embodiments, the first medium comprises animal or human proteins, such as animal or human cord blood plasma, and/or PRP lysate. In some embodiments, the second medium does not comprise animal or human proteins. In some embodiments, the second medium comprises cytokines, biologically active proteins, ITS, FGF, VEGF, IGF-1 and/or glucose. In some embodiments, the second medium does not comprise animal or human proteins, and comprises cytokines, biologically active proteins, ITS, FGF, VEGF, IGF-1 and/or glucose.

The present invention thus allows to expand and highly enrich a MSC culture for Muse cells. This has several advantages, because Muse cells exhibit unlimited levels of pluripotency in all three germlines, yet in contrast to MSCs do not produce teratomas or other tumors. In fact, the EVs obtained from Muse cells have anti-inflammatory effect that is believed to be, at least in part, due to their secretome and/or EVs. The inventors surprisingly found that this is indeed the case, and that Muse cells as well as EVs from said Muse cell have a strong anti-inflammatory effect that is at least as potent and last longer than the corticosteroid dexamethasone. The EVs, such as EV100 and EV500, produced according to the inventive method have anti-inflammatory properties.

In a fourth aspect, the invention relates to a plurality of EVs obtained or obtainable by the method according to the third aspect. Throughout the application, “obtainable” and “obtained” are used interchangeably.

### Coating of Plates

Adherent cultures of MSCs and Muse cells is performed on coated dishes, such as culture dishes and flasks coated with collagen, poly-Lysine, gelatin, Matrigel, or a combination thereof.

The Muse cells may be cultured as 2D or 3D cultures according to routine methods.

The EVs, such as EV100 or EV500, may be further purified, concentrated and/or lyophilized. The separation of EV100 and EV500 is done by routine methods based on their different size. Methods include, but are not limited to, gel chromatography, differential centrifugation, tangential filtration, and ultrafiltration. As an example, after removing the cellular debris by a brief centrifugation step after the harvest, the secretome comprising the EV500 fraction can be centrifuged at 12,000 g. The EV100 will remain in the supernatant, while the EV500 fraction will be pelleted.

The EV100 fraction can be further subjected to a filtration step with a predetermined pore size, e.g. 220 μm to obtain a EV100 fraction comprising exosomes with a defined size, i.e. about 30 to 220 μm.

The pellet comprising EV500 vesicles can be resuspended to obtain EV500 fraction. Alternatively, the EV500 can be lyophilized and stored indefinitely. In some embodiments, the EV500 is resuspended in a lyophilization buffer.

Alternatively, to the centrifugation based method above, EV100 and EV500 can be separated by gel filtration. The hydrophobic particles with a size of 220-500 nm will be obtained. To further separate the EV100s from the remaining secretome components, the EV100 fraction is processed through gel filtration. The final EV100 fraction is filtered through a 220 nm membrane with the final fraction having a mean diameter of about 100 nm.

The above methods to separate EV100 and EV500 are for illustration purposes and are not meant to be limiting in any manner. The method provided herein results in the production and isolation of secretomes from high muse cell content MSCs and the separation of EV100 and EV500 particles from the secretome. In addition, these steps prepare them for storage through freezing in suspension, or lyophilization.

Alternatively, or in addition thereto, EV100 and EV500 may further be identified and separated from each other by using antibodies, aptamers, aptamer analogs, or molecularly imprinted polymers specific for a desired surface antigen that is expressed in one of the fractions, i.e. EV100 or EV500.

### 3D Culture

The invention provides a method for preparing EVs derived from Muse cells, said method comprising
(a) providing Muse cells;
(b) culturing the Muse cells of (a) in a first medium for a time period sufficient to expand Muse cells in an undifferentiated state;
(c) culturing the Muse cells of (c) in a second medium for a time period sufficient to induce production of EVs, wherein the EVs are released into the second medium; and
(d) harvesting the EVs for one or more times, wherein each harvesting comprises collecting the second medium comprising the EVs to obtain a harvest.

Steps (b) and (c) of the method may be performed on coated dishes or in a bioreactor.

In one embodiment, step (b) of the method is performed in a bioreactor. In another embodiment, steps (b) and (c) of the method are performed in a bioreactor.

A bioreactor is a system designed to grow and/or culture cells or tissues submerged in a liquid medium. The culture may be a suspension culture. The mode of operation of the bioreactor may be batch, fed batch or continuous. A bath culture refers to a large-scale culture in which cells are grown in a fixed volume of culture medium. A fed batch culture refers to a large-scale culture in which one or more nutrients are fed to the bioreactor during culture. Continuous culture refers to a bioreactor culture in which nutrients are continuously added and/or removed. Bioreactor cultures may progress from batch, to fed batch, to continuous.

In one embodiment, step (b) was performed in a bioreactor with interval agitation. Interval agitation may be performed by alternating 1-60 min without agitation followed by 60-360 min with agitation. In one embodiment, interval agitation comprises cycles of 5-15 min without agitation followed by 120-180 min with agitation. In a preferred embodiment, interval agitation comprises cycles of 10 min without agitation followed by 150 min with agitation.

In one embodiment, step (c) is performed under continuous agitation.

In one embodiment, the method is performed at a large scale. The large scale culture is performed in a bioreactor. The bioreactor has a capacity of at least 1 L, at least 2 L, at least 5 L, at least 10 L, at least 15 L, at least 20 L, at least 30 L, at least 40 L, at least 50 L, at least 100 L, at least 200 L, at least 500 L or at least 1000 L.

The large scale culture is performed at a volume of at least 1 L of medium. In one embodiment, the large scale culture comprises at least 1 L. at least 2 L, at least 5 L, at least 10 L, at least 15 L, at least 20 L, at least 30 L, at least 40 L, at least 50 L, or at least 100 L of culture medium.

In one embodiment, step (a) of the method is performed on coated plates and steps (b) and (c) of the method are performed in a bioreactor.

In one embodiment, the culture is a 3D scaffold culture.

In one preferred embodiment, the culture is a microcarrier culture. A three dimensional microcarrier culture uses coated microcarriers cultured in suspension on which cells attach and grow.

Microcarriers may be microporous, macroporous or non-porous microcarriers. Microcarriers may be coated with one or more suitable coating substances such as dextran, gelatin, cellulose, agarose, alginate, collagen, protein, fibronectin, casein, chitosan or Poly(N-isopropylacrylamide). In a preferred embodiment, microcarriers are coated with L-Lysine or collagen. In a particularly preferred embodiment, microcarriers are coated with collagen. In another embodiment, the culture is a suspension culture. Muse cells may form free floating aggregates (i.e. spheroids) in suspension culture.

### Characterization and Quantification of Muse Cells

Muse cells are cultured in a mixed culture, wherein the method according to the invention enriches the proportion of Muse cells.

Muse cells are cultured in undifferentiated state which is characterized by expression of SSEA-3. In addition, in some embodiments, Muse cells express one or more markers that are common to MSCs and stem cells in general, such as CD90, CD73, CD105, NANOG, Oct-4, Sox-2, Klf4 or c-Myc. Muse cells may further be identified by their lack of CD45 expression.

In some embodiments, Muse cells express SSEA-3 and CD90. In some embodiments, Muse cells express SSEA-3 and CD73. In some embodiments, Muse cells express SSEA-3 and CD105. In some embodiments, Muse cells express SSEA-3, CD90, CD73 and CD105. In some embodiments, the Muse cells express SSEA-3, CD90 and one, two, three or more markers selected from CD73, CD105, NANOG, Oct-4, Sox-2, Klf4 or c-Myc. Muse cells may further be identified by their lack of CD45 expression.

The expression of one or more of these markers can be detected by Fluorescent activated cell sorting (FACS) or magnetic activated cell sorting (MACS). In some embodiments, the co-expression of these markers on Muse cells is determined.

In some embodiments, the relative concentration of Muse cells prior to enrichment, during enrichment, and/or after enrichment is determined.

In some embodiments, the Muse cells are isolated by FACS or MACS by using the appropriate antibodies. In some embodiments, the isolated Muse cells are purified and processed for administration.

### Enrichment of Muse Cells

The method of the invention provides for an enrichment of the Muse cells.

In some embodiments, the Muse cells that are initially present in concentrations below 2%, <1.5%, <1%, or about 0.1 to 1% are enriched to comprise at least 10% Muse cells. The inventive method greatly expands the cell numbers and enriches for Muse cells, resulting in an increase of absolute Muse cell numbers, but also an increase in the relative concentration in the mixed culture.

In some embodiments, the Muse cells are expanded at least 10, 100, 10³, 10⁴, 10⁵-fold relative to the concentration of Muse cells in the tissue the Muse cells were obtained from. In some embodiments, the Muse cells are enriched and are present in step(c) of the provided method in a concentration of at least 10%, 15%, 20%, 30%, 35%, 40%, 45%, 50%, 55%, 60%, 65%, 70%, 80% or more. In some embodiments, the Muse cells in step(c) of the provided method are present in a concentration of at least 20%. In some embodiments, the Muse cells in step(c) of the provided method are present in a concentration of at least 30%. In some embodiments, the Muse cells in step(c) of the provided method are present in a concentration of about 10 to 60%. In some embodiments, the Muse cells in step(c) of the provided method are present in a concentration of about 20-50%. In some embodiments, the Muse cells in step(c) of the provided method are present in a concentration of at least 40%. In some embodiments, the Muse cells in step(c) of the provided method are present in a concentration of at least 45%. In some embodiments, the Muse cells in step(c) of the provided method are present in a concentration of at least 50%.

### Pharmaceutical Composition of the Invention

In a fifth aspect, the invention relates to a pharmaceutical composition, hereinafter pharmaceutical composition of the invention, comprising EVs, such as EV100, EV500 or both, according to the first or fourth aspects, or a composition comprising said EVs according to the second aspect.

The term “pharmaceutical composition”, as used herein, refers to a composition comprising a therapeutically effective amount of the agent according to the present invention, i.e., the plurality of EVs of the first or fourth aspects, or the composition of the second aspect, and at least one pharmaceutically acceptable carrier.

The terms “pharmaceutically acceptable carrier”, or “pharmaceutically acceptable excipient,” “pharmaceutically acceptable diluent,”, or “pharmaceutically acceptable vehicle,” used interchangeably herein, refer to a non-toxic solid, semisolid or liquid filler, diluent, encapsulating material or formulation auxiliary of any conventional type. A pharmaceutically acceptable carrier is essentially non-toxic to recipients at the dosages and concentrations employed and is compatible with other ingredients of the formulation. A pharmaceutically acceptable carrier will not inhibit otherwise adversely affect the function of the agent according to the present invention. Suitable carriers include, but are not limited to water, dextrose, glycerol, saline, ethanol, and any combination thereof. The carrier can contain additional agents such as wetting or emulsifying agents, pH buffering agents, or adjuvants, which enhance the effectiveness of the formulation.

The person skilled in the art will appreciate that the nature of the excipient in the pharmaceutical composition of the invention will depend to a great extent on the administration route. In the case of the pharmaceutical compositions formulated for their oral (or topical) use, a pharmaceutical composition according to the invention normally contains the pharmaceutical composition of the invention mixed with one or more pharmaceutically acceptable excipients. These excipients can be, for example, inert fillers or diluents, such as sucrose, sorbitol, sugar, mannitol, microcrystalline cellulose, starches, including potato starch, calcium carbonate, sodium chloride, lactose, calcium phosphate, calcium sulfate or sodium phosphate; crumbling agents and disintegrants, for example cellulose derivatives, including microcrystalline cellulose, starches, including potato starch, sodium croscarmellose, alginates or alginic acid and chitosans; binding agents, for example sucrose, glucose. sorbitol, acacia, alginic acid, sodium alginate, gelatin, starch, pregelatinized starch, microcrystalline cellulose, aluminum magnesium silicate, sodium carboxymethylcellulose, methylcellulose, hydroxypropyl methylcellulose, ethylcellulose, polyvinylpyrrolidone, polyvinyl acetate or polyethylene glycol, and chitosans; lubricating agents, including glidants and antiadhesive agents, for example magnesium stearate, zinc stearate, stearic acid, silicas, hydrogenated vegetable oils or talc.

In a particular preferred embodiment, the pharmaceutical compositions of the invention is formulated for administration via the rectal, nasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir. Pharmaceutical compositions according to the invention can be prepared, for instance, as injectables such as liquid solutions, suspensions, and emulsions.

In some embodiments, the pharmaceutical composition comprises EV100 and a pharmaceutically acceptable carrier, optionally further comprising an active agent. In some embodiments, the pharmaceutical composition comprises EV500 and a pharmaceutically acceptable carrier, optionally further comprising an active agent. In some embodiments, the pharmaceutical composition comprises EV100 and EV500 and a pharmaceutically acceptable carrier, optionally further comprising an active agent. The active agent may be any agent that further support or improves or acts in concert with the EVs comprised in the composition to bring about the desired therapeutic effect.

In some embodiments, the pharmaceutical composition of the invention comprises at least 10⁵, 10⁶, 10⁷, 10⁸, 10⁹ 10¹⁰, 10¹¹ EVs, such as EV100 or EV500 or a combination thereof.

### Methods of Treatment

### Inflammation or an Inflammatory Disorder

In a sixth aspect, the invention relates to the pharmaceutical composition of the invention for use in therapy. In a related aspect, the invention relates to the pharmaceutical composition of the invention for use in a method of treating inflammation or an inflammatory disorder in a subject in need thereof, said method comprising administering a therapeutically effective amount of said composition to said subject.

The term “inflammation” and “inflammatory disorder” are used interchangeably and refer to any condition or disease that is characterized by antiinflammation, which may result from, or be triggered by, a deregulation of the normal immune response. Because inflammation mediates and is the primary driver of many medical and autoimmune disorders, within the context of the present invention, the term “inflammatory disorder” is also meant to encompass autoimmune disorders and inflammatory diseases.

The term “autoimmune disorder”, as used herein, refers to a condition in a subject characterized by cellular, tissue and/or organ injury caused by an immunological reaction of the subject to its own cells, tissues and/or organs. Illustrative, non-limiting examples of autoimmune diseases which can be treated with the methods or pharmaceutical compositions of the invention include alopecia areata, rheumatoid arthritis (RA), ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, sarcoidosis, scleroderma, progressive systemic sclerosis, Sjogren's syndrome, Good pasture's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, Wegener's granulomatosis, anti-glomerular basement membrane disease, antiphospholipid syndrome, autoimmune diseases of the nervous system, familial mediterranean fever, Lambert-Eaton myasthenic syndrome, sympathetic ophthalmia, polyendocrinopathies, psoriasis, etc.

The term “inflammatory disease”, as used herein, refers to a condition in a subject characterized by inflammation, e.g. chronic inflammation. The inflammation may be an acute or chronic inflammation or inflammatory disorder or disease. Illustrative, non-limiting examples of inflammatory disorders include, but are not limited to rheumatoid arthritis, Osteoarthritis, Type 2 diabetes mellitus, Type 1 diabetes mellitus, inflammatory bowel disease, asthma, endometriosis, cancer, alopecia, inflammatory arthritis, psoriatic arthritis, lupus, juvenile idiopathic arthritis, gout, systemic lupus erythematosus, and seronegative spondyloarthropathies, relapsing polychondritis neurological inflammation, multiple sclerosis, migraine, Traumatic Brain Injury (TBI) and Chronic Traumatic Encephalopathy (CTE), Celiac Disease, Inflammatory Bowel Disease (IBD), encephalitis, chronic obstructive pulmonary disease (COPD), inflammatory osteolysis, Crohn's disease, ulcerative colitis, allergic disorders, septic shock, pulmonary fibrosis (e.g., idiopathic pulmonary fibrosis), inflammatory vacultides (e.g., polyarteritis nodosa, Wegner's granulomatosis, Takayasu's arteritis, temporal arteritis, and lymphomatoid granulomatosus), post-traumatic vascular angioplasty (e.g. restenosis after angioplasty), undifferentiated spondyloarthropathy, undifferentiated arthropathy, arthritis, inflammatory osteolysis, chronic hepatitis, chronic inflammation resulting from chronic viral or bacterial infections, and acute inflammation, such as sepsis.

In one embodiment, the inflammatory disease comprises rheumatoid arthritis (RA), Osteoarthritis, Type 2 diabetes mellitus, Type 1 diabetes mellitus, inflammatory bowel disease, asthma, endometriosis, cancer, alopecia, inflammatory arthritis, psoriatic arthritis, psoriasis, lupus, juvenile idiopathic arthritis, gout, systemic lupus erythematosus, and seronegative spondyloarthropathies, relapsing polychondritis neurological inflammation, multiple sclerosis, migraine, Traumatic Brain Injury (TBI) and Chronic Traumatic Encephalopathy (CTE), Covid-19, long Covid-19, Celiac Disease, Inflammatory Bowel Disease (IBD), encephalitis, chronic obstructive pulmonary disease (COPD), inflammatory osteolysis, Crohn's disease, ulcerative colitis, allergic disorders, septic shock, pulmonary fibrosis (e.g., idiopathic pulmonary fibrosis), inflammatory vacultides (e.g., polyarteritis nodosa, Wegner's granulomatosis, Takayasu's arteritis, temporal arteritis, and lymphomatoid granulomatosus), post-traumatic vascular angioplasty (e.g. restenosis after angioplasty), undifferentiated spondyloarthropathy, undifferentiated arthropathy, arthritis, inflammatory osteolysis, chronic hepatitis, chronic inflammation resulting from chronic viral or bacterial infections, and acute inflammation, such as sepsis.

In a particular embodiment, the inflammatory disorder is selected from the group consisting of rheumatoid arthritis, Osteoarthritis, Type 2 diabetes mellitus, Type 1 diabetes mellitus, inflammatory bowel disease, asthma, endometriosis, cancer, alopecia, inflammatory arthritis, psoriatic arthritis, lupus, juvenile idiopathic arthritis, gout, systemic lupus erythematosus, and seronegative spondyloarthropathies, relapsing polychondritis neurological inflammation, multiple sclerosis, migraine, Traumatic Brain Injury (TBI) and Chronic Traumatic Encephalopathy (CTE).

In a preferred embodiment, the inflammatory disorder is selected from the group consisting of rheumatoid arthritis, Osteoarthritis, cancer, alopecia, inflammatory arthritis, neurological inflammation, Covid-19 and long Covid-19.

The term “a therapeutically effective amount” refers to an amount sufficient to produce a desired therapeutic effect, e.g., ameliorating the severity of a symptom of a disease. In a particular embodiment, the therapeutically effective amount can be the amount to reduce inflammation.

The Muse cells from which the EVs are derived can be autologous, allogeneic or xenogeneic. As used herein, the term “autologous” means that the donor of the Muse cells and the recipient of the EVs (e.g. EV100, EV500 or combination thereof), derived from said Muse cells are the same subject. The term “allogeneic” means that the donor of the Muse cells and the recipient of the EVs (e.g. EV100, EV500 or combination thereof) derived from said Muse cells are different subjects. The term “xenogeneic” means that the donor of the Muse cells and the recipient of the EVs (e.g. EV100, EV500 or combination thereof) derived from said Muse cells are subjects of different species. In a preferred embodiment, the Muse cells from which the EVs (e.g. EV100, EV500 or combination thereof) derived are allogeneic.

In a particular embodiment, the EVs (e.g. EV100, EV500 or combination thereof) or pharmaceutical composition of the invention is administered systemically or locally. The term “systemically” means that the EVs (e.g. EV100, EV500 or combination thereof) or pharmaceutical composition of the invention may be administered to a subject in a non-localized manner. The systemic administration of the EVs (e.g. EV100, EV500 or combination thereof) or pharmaceutical composition of the invention may reach several organs or tissues throughout the body of the subject or may reach specific organs or tissues of the subject. The term “locally administered”, as used herein, means that the EVs (e.g. EV100, EV500 or combination thereof) or pharmaceutical composition of the invention may be administered to the subject at or near a specific site. In a more particular embodiment, the EVs (e.g. EV100, EV500 or combination thereof) is administered via intravenous, intramuscular, subcutaneous, rectal, vaginal, inhalation, oral, buccal, sublingual, transdermal, ocular, intrathecal or nasal administration.

In a particular embodiment, the EVs (e.g. EV100, EV500 or combination thereof) is administered in conjunction with at least one additional therapeutic agent. The term “therapeutic agent”, as used herein, refers to an agent useful in the treatment of a disease. In a particular embodiment, the additional therapeutic agent is a known drug for the treatment of the particular inflammation or inflammatory disorder present in the subject. Exemplary additional therapeutic agents comprise but are not limited to corticosteroids or non-steroidal anti-inflammatory compounds.

The expression “administered in conjunction” means that the EVs (e.g. EV100, EV500 or combination thereof) or pharmaceutical composition of the invention can be administered jointly or separately, simultaneously, at the same time or sequentially with the additional therapeutic agent, for example a therapeutic useful in the treatment of a disease associated with inflammation, in any order. For example, the administration of the EVs (e.g. EV100, EV500 or combination thereof) or pharmaceutical composition of the invention can be done first, followed by the administration of one or more additional therapeutic agents; or the administration of the EVs (e.g. EV100, EV500 or combination thereof) or pharmaceutical composition of the invention can be done last, preceded by the administration of one or more additional therapeutic agents; or the administration of the EVs (e.g. EV100, EV500 or combination thereof) or pharmaceutical composition of the invention can be done at the same time as the administration of one or more additional therapeutic agents.

The EVs (e.g. EV100, EV500 or combination thereof) or the pharmaceutical composition of the invention have a strong and prolonged anti-inflammatory effect (Example 11).

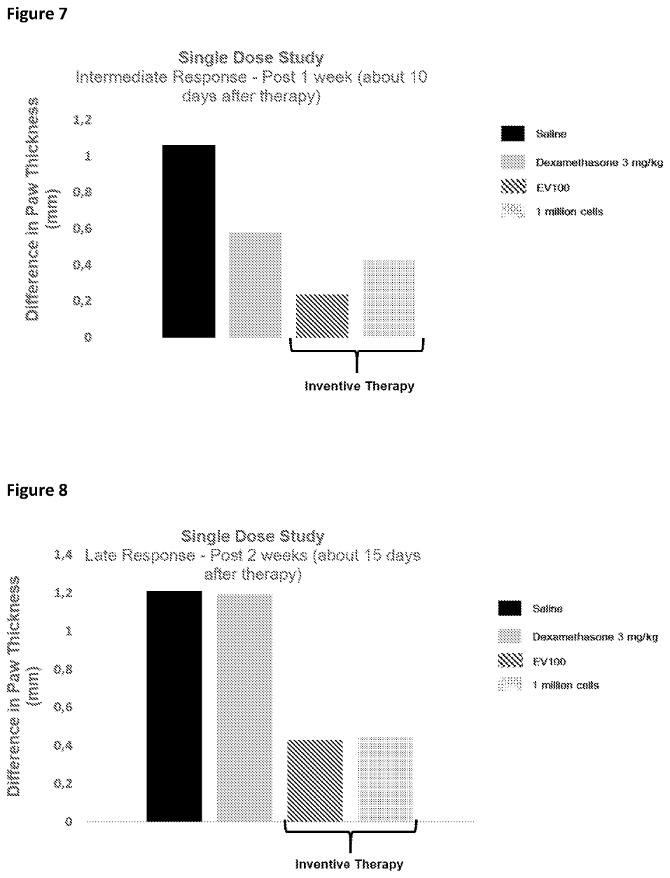

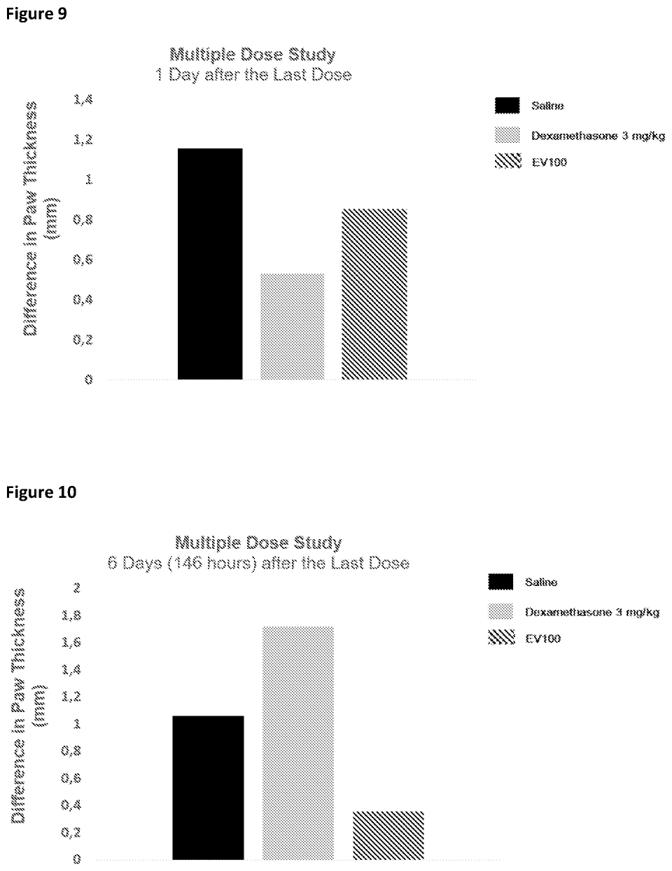

The anti-inflammatory effect was assessed in Example 11 using the paw edema model, which induces an inflammatory response by injection of carrageenan into the paw of an animal. Thirty minutes after induction of the inflammatory response, the animals were treated with either saline (negative control), dexamethasone (positive control), EV100 or Muse cell enriched cells. Interestingly, only EV100 and the Muse cells inhibited the inflammatory response (as measured by the decrease in paw thickness) almost instantaneously. The immediate response thus precedes any effect that can be exerted by dexamethasone, which at the same time point was indistinguishable from the saline control (FIG. 5). One day after treatment, dexamethasone, EV100 and Muse cell treatments resulted in a decrease of the inflammation, which was further enhanced after one week post treatment (FIGS. 6 and 7). Surprisingly, the effect of EV100 and Muse cells was stable even after 2 weeks after the single injection (FIG. 8), wherein the dexamethasone treated animals were indistinguishable from the saline treated controls. The treatment with the established anti-inflammatory agent dexamethasone thus began later and lasted for a shorter period than the single administration of EV100 and Muse cells. This late effect of further confirmed with repeated injections, wherein EV100 was capable of successfully treating the inflammation to great extent (FIGS. 9-11) again demonstrating that it is particularly well-suited for long-term treatments.

The anti-inflammatory effect was confirmed in Example 16 using the LPS-induced sepsis model. LPS injection induces sepsis in rodents that shares many similarities with the initial phase of human sepsis, such as extensive pro-inflammatory cytokine production including iFNgamma, IL-1beta, and IL-6.

In a first experiment, acute inflammatory changes in mice after injection of LPS were evaluated. The time-points 6 h and 24 h after LPS injection were chosen for further analysis. Mice were divided into three groups: PBS treated (LPS+PBS), high Muse cell MSC exosome treated (LPS+Exo) and a positive control group treated with dexamethasone (LPS+Dex).

The inventors were able to show that 24 h after LPS injection, treatment with high Muse cell MSC exosomes decreased pro-inflammatory cytokines compared to control conditions. Interestingly, treatment with high Muse MSC cells rather than high Muse MSC exosomes exacerbated inflammatory markers as well as sepsis symptoms in mice (data not shown). Thus, high Muse cell MSC exosomes outperformed treatment with high Muse cell MSCs in a model of inflammation.

In one particular embodiment, treatment with the EVs (e.g. EV100, EV500 or combination thereof) or the pharmaceutical composition comprising the EVs obtained from Muse cells or Muse cell-enriched MSC cultures of the invention reduces secretion of inflammatory cytokines. For example, in one embodiment, treatment with the EVs (e.g. EV100, EV500 or combination thereof) or the pharmaceutical composition of the invention modulates secretion of one or more cytokines selected from the group existing of INF-γ, IL-1β, IL-6, and IL-10.

In one embodiment, treatment with the EVs (e.g. EV100, EV500 or combination thereof) or the pharmaceutical composition of the invention decreases secretion of one or more pro-inflammatory cytokines. In one embodiment, treatment with the EVs (e.g. EV100, EV500 or combination thereof) or the pharmaceutical composition of the invention decreases secretion of one or more pro-inflammatory cytokines selected from the group existing of INF-γ, IL-1β, and IL-6. In one embodiment, treatment with the EVs (e.g. EV100, EV500 or combination thereof) or the pharmaceutical composition of the invention decreases secretion of one or more pro-inflammatory cytokines selected from INF-γ or IL-1β. In one embodiment, treatment with the EVs (e.g. EV100, EV500 or combination thereof) or the pharmaceutical composition of the invention decreases secretion of INF-γ. In one embodiment, treatment with the EVs (e.g. EV100, EV500 or combination thereof) or the pharmaceutical composition of the invention decreases secretion of IL-1β.

Cytokines are considered to be pain mediators in neurovascular inflammation. Furthermore, cytokine receptors are widely expressed in the central nervous system by all cell types, including neurons, indicating that cytokines may be a cause of migraine pain [1].

Hence, in one embodiment, the invention also provides EVs (e.g. EV100, EV500 or combination thereof) or the pharmaceutical composition for use in treating migraine.

Furthermore, cytokine-mediated signaling pathways are central to the pathogenesis of rheumatoid arthritis, particularly IL-6 and TNF-α [2]. Hence, in a particular embodiment, the invention also provides EVs (e.g. EV100, EV500 or combination thereof) or the pharmaceutical composition for use in treating rheumatoid arthritis.

Cytokines also play a role in inflammatory skin conditions such as psoriasis. Psoriasis has been shown to be a product of cytokine storm, and drugs targeting TNF-α, interleukins, other cytokine and factors downstream of cytokine receptors are currently undergoing clinical trials for psoriasis [3].

Hence, the invention also provides EVs (e.g. EV100, EV500 or combination thereof) or the pharmaceutical composition for use in treating psoriasis.

Covid 19 has been strongly associated with cytokines. It has been shown that Interleukins (such as IL-1, IL-4, IL-6, IL-7, IL-10, IL-12, IL-17, and IL-18), IFN-γ, TNF-α, TGF-β and NF-κB play major roles in the body's inflammatory response to SARS-CoV-2 infection [4]. Furthermore, pro-inflammatory cytokines contribute to clinical symptoms of Covid 19, particularly acute respiratory distress syndrome (ARDS). ARDS is triggered by cytokine storm, and cytokines such as TNF-α, IL-1 and IL-6 have been shown to play a significant role in ARDS [5]. In another embodiment, the invention also provides EVs (e.g. EV100, EV500 or combination thereof) or the pharmaceutical composition for use in treating Covid 19.

Inflammatory mediators such as IL-1β, IL-6, IL-6, IL-17, IFN-γ, matrix metalloproteinase-2 (MMP-2), MMP-9, and TNF-α also contribute to the inflammatory phenotype of rosacea [6].

Hence, in another embodiment, the invention also provides EVs (e.g. EV100, EV500 or combination thereof) or the pharmaceutical composition for use in treating rosacea.

Drugs with an anti-inflammatory action are also being investigated for their potential as therapeutic agents in melasma, since several interleukins and cytokines can stimulate melanocyte proliferation, upregulate melanin production, and enhance melanosome transfer [7].

Thus, in another embodiment, the invention also provides EVs (e.g. EV100, EV500 or combination thereof) or the pharmaceutical composition for use in treating melasma.

Recent studies also suggest that neuroinflammation associated with cytokine secretion following nerve damage may also play a role in the pathogeneisis of neuropathic pain. In a recent study of diabetic neurophathy, basement levels of TNF-α and IL-6 were higher in the neuropathic group [8].

In another embodiment, the invention also provides EVs (e.g. EV100, EV500 or combination thereof) or the pharmaceutical composition for use in treating diabetic neuropathy.

Chronic low-level inflammation is also associated with cellular senescence and aging phenotypes, including skin aging. Permanent senescence or skin aging can be induced in nonreplicating (senescent) fibroblasts, the increase of which results in the production of SASPs rich in pro-inflammatory cytokines, including interleukin (IL)-1, IL-6, IL-8, IL-18, matrix metalloproteinases (MMPs), and a variety of other inflammatory chemokines [9].

In another embodiment, the invention also provides EVs (e.g. EV100, EV500 or combination thereof) or the pharmaceutical composition for use in treating or preventing fibroblast senescence.

The strong anti-inflammatory effect of EVs, such as EV100, EV500 (data not shown) and Muse cells (data not shown), lack of an immunogenic reaction, and in conjunction with the lack of the adverse effects associated with use of corticosteroids, make Muse cells and EVs obtained from Muse cells very promising tools for numerous therapeutic applications.

### Cancer or Tumor

In a further aspect, the invention relates to the pharmaceutical composition of the invention for use in a method of treating a tumor or cancer in a subject in need thereof, said method comprising administering a therapeutically effective amount of the pharmaceutical composition to the subject.

The terms “tumor” and “cancer” have been previously defined.

In a particular embodiment, the EVs (e.g. EV100, EV500 or combination thereof) or pharmaceutical composition of the invention is administered to up-regulate an immune response and enhance the tumor targeting of the subject's immune system. In some embodiments, the cancer being treated is characterized by infiltration of leukocytes (T-cells, B-cells, macrophages, dendritic cells, monocytes) into the tumor microenvironment, or so-called “hot tumors” or “inflammatory tumors”. In some embodiments, the cancer being treated is characterized by low levels or undetectable levels of leukocyte infiltration into the tumor microenvironment, or so-called “cold tumors” or “non-inflammatory tumors”. In some embodiments, the composition is administered in an amount and for a time sufficient to convert a “cold tumor” into a “hot tumor”, e.g., said administering results in the infiltration of leukocytes (such as T-cells) into the tumor microenvironment.

In some embodiments, the EVs (e.g. EV100, EV500 or combination thereof) or pharmaceutical composition of the invention is administered to the subject in a therapeutically effective amount to slow down tumor progression, reduce the size of the tumor and/or ameliorate adverse effects associated with the presence of the tumor or cancer.

In preferred embodiments, the cancer is a cancer of the breast, liver, pancreas, lymph nodes, lung, skin, bone marrow, adipose tissue, nerve tissue, glioma, stomach, colon, blood, kidney, or endometrium. cancer of the breast, liver, pancreas, lymph nodes, lung, skin, bone marrow, adipose tissue, nerve tissue, glioma, stomach, colon, blood, kidney, or endometrium.

In a particular embodiment, the EVs (e.g. EV100, EV500 or combination thereof) or pharmaceutical composition of the invention is administered in conjunction with at least one additional therapeutic agent, wherein the therapeutic agent refers to an anti-cancer agent.

Exemplary additional therapeutic agents for use in a method of treating cancer or tumor comprise but are not limited to alkylating agents, nitrosoureas, anti-metabolites, plant alkaloids, anti-tumor antibiotics, hormonal agents and/or corticosteroids.

In a particular embodiment, the additional therapeutic agent is cancer agent is an alkylating agent, antimetabolite, hormone, miRNA, chemotherapeutic agent, or cytotoxic agent. In a preferred embodiment, the additional therapeutic anti-cancer agent is a therapeutic miRNA, therapeutic IncRNA protein, chemotherapeutic agent, anti-mitotic agent, therapeutic antibody or fragment thereof.

In a related aspect, the EVs (e.g. EV100, EV500 or combination thereof) or pharmaceutical composition of the invention is used for the manufacture of a medicament.

### Restoring Health, Regeneration

In a further aspect, the invention relates to the pharmaceutical composition of the invention for use in a method of restoring health in a subject in need thereof, said method comprising administering a therapeutically effective amount of the pharmaceutical composition to the subject.

In a further aspect, the invention relates to the pharmaceutical composition of the invention for use in a method of regeneration in a subject in need thereof, said method comprising administering a therapeutically effective amount of the pharmaceutical composition to the subject.

In some embodiments, the EVs (e.g. EV100, EV500 or combination thereof) or pharmaceutical composition of the invention is administered to the subject in a therapeutically effective amount to modulate epigenetic marks present in the subject, that adversely affect health or the ability to regenerate in the subject. In a particular embodiment, the regeneration comprises reversing the status of one or more epigenetic marks to a status correlated with a subject of younger age.

In some embodiments, the subject in need of regeneration or restoration of health suffers from stroke, myocardial infarction, age-related cognitive decline, or age-related loss of motor function. In some embodiments, the subject is above the age of 50, 60, 70 or 80 years. In a particular embodiment, the subject is above the age of 50 years, and characterized by the presence of one or more epigenetic marks at predetermined sites that are associated with degeneration with degeneration or negative impacts on health. Epigenetic marks may include DNA methylation and histone modifications.

### Wound Healing

In some embodiments, the EVs (e.g. EV100, EV500 or combination thereof) or pharmaceutical composition of the invention is administered to the subject in a therapeutically effective amount to promote wound healing.

The term “promoting wound healing” refers to improving, increasing, or inducing closure, healing, or repair of a wound. Wound healing is considered to be promoted, for example, if the time of healing a wound treated with EVs (e.g. EV100, EV500 or combination thereof) or pharmaceutical composition of the invention compared to a wound not treated with EVs (e.g.

EV100, EV500 or combination thereof) or pharmaceutical composition of the invention is decreased by about 10%, such as decreased by about 25%, such as decreased by about 50%, such as decreased by about 75%.

The EVs (e.g. EV100, EV500 or combination thereof) or pharmaceutical composition of the invention may be applied in any suitable quantity to effect the desired result of wound healing. In particular embodiments, the topical administration of the EVs, such as EV100 or EV500, or pharmaceutical composition of the invention results in the faster or improved epithelization of a wound bed in a subject in need thereof.

EVs Loaded with Cargo

In a further aspect, the EVs (e.g. EV100, EV500 or combination thereof) of the invention can be used to deliver a cargo, such as a therapeutic agent or modulatory agent, to a target site.

The term “target site” refers to any site that is a site of injury or in need of repair.

The term “cargo” as used herein refers to any type of molecule or any type of RNA (miRNA, mRNA, tRNA, rRNA, siRNA, iRNA, regulating RNA, gRNA, long interference RNA, non-coding and coding RNA); any type of DNA (DNA fragments, DNA plasmids, iDNA); including any type of nucleic acid including antisense oligonucleotides (ASO); any genetic material; any genetic construct; any nucleic acid construct; any plasmid or vector; any protein including recombinant endogenous protein, enzyme, antibody, wnt signaling proteins; any lipid; any therapeutic molecule or diagnostic molecule; any cellular component; chimeric antigen receptor T cell (CAR-T cell) transduced without using retroviruses; any virus including retrovirus, adenoviruses (AdV), adeno-associated viruses (AAV) of any variety and strain, and DNA viruses; any gene editing technology including clustered regularly interspaced short palindromic repeats (CRISPR), CRISPR/Cas 9 system, any endonucleases for base editing, a Zinc finger, a single base editor, Transcription activator-like effector nucleases (TALENs), any meganuclease; any synthetic molecular conjugate; or combination thereof loaded into an EVs (e.g. EV100, EV500 or combination thereof). Typically, such cargo is naturally not present in the EVs.

In some embodiments, the cargo can be a therapeutic agent, such as a pharmaceutical drug, anti-inflammatory agent, or anti-tumor agent. The administration of the EVs or pharmaceutical composition coupled with such a therapeutic cargo would thus increase the therapeutic effect of the EVs or pharmaceutical composition.

### Non-Therapeutic Uses

In addition to the therapeutic uses, the EVs and pharmaceutical composition of the invention can be used in various non-therapeutic applications, including, but not limited to improving the appearance of wrinkles of the skin of a subject, and preventing hair loss or promoting hair growth in a subject.

### Dosage Forms

The pharmaceutical composition of the present invention is not limited to, but may be used in the form of oral dosage forms such as powders, granules, capsules, tablets, aqueous suspensions, external preparations, microneedle patches, suppositories, and sterile injection solutions, respectively, according to a conventional method can be used. The pharmaceutical composition of the present invention may include a pharmaceutically acceptable carrier.

Pharmaceutically acceptable carriers may include binders, lubricants, disintegrants, excipients, solubilizers, dispersants, stabilizers, suspending agents, pigments, fragrances, etc., when administered orally, and in the case of injections, buffers, preservatives, painless Agents, solubilizers, isotonic agents, stabilizers, etc. can be used in combination, and for topical administration, bases, excipients, lubricants, preservatives, etc. can be used. The formulation of the pharmaceutical composition of the present invention can be variously prepared by mixing with a pharmaceutically acceptable carrier as described above. For example, when administered orally, tablets, troches, capsules, elixirs, suspensions, syrups, wafers, etc., may be prepared in the form of injections, and in the case of injection, unit dosage ampoules or multiple dosages may be prepared. Others can be formulated as solutions, suspensions, tablets, capsules, and sustained release preparations. The skilled person will know which dosage form is suitable for the desired administration. For example, for nasal administration, the EVs or pharmaceutical composition would be provided as an aerosol, in liquid form or an ointment or gel, but not as a capsule or tablet.

Pharmaceutical composition of the invention may be in provided in solid, semi-solid, or liquid dosage forms comprising tablet, capsule, gel-capsule, aerosol, liquid solution or suspension, injectable and infusion solution, eye-drop, film, cream, gel, film, and ointment. The dosage forms may be further provided as a lyophilized preparation or resuspended solution.

### Modes of Administration

The EVs of the invention, such as EV100 or EV500, can be administered to a subject by any appropriate route. Suitable routes of administration include rectal, intranasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir into the subject. It will be apparent to the practitioner, which administration route is most suitable depending on the site or organ to be treated, and disease to be treated.

The compositions of the invention can be administered by one of many routes, depending on the embodiment. For example, the EVs or the pharmaceutical composition of the invention may be administered by local or systemic administration. Local administration, depending on the tissue to be treated, may in some embodiments be achieved by direct administration to a tissue (e.g., direct injection, such as intramyocardial injection). Local administration may also be achieved by, for example, lavage of a particular tissue (e.g., intra-intestinal or peritoneal lavage). In several embodiments, systemic administration is used and may be achieved by, for example, intravenous and/or intra-arterial delivery. In certain embodiments, intracoronary delivery is used. In several embodiments, the EVs of the invention are specifically targeted to the damaged or diseased tissues. In some such embodiments, the EVs are modified (e.g., genetically or otherwise) to direct them to a specific target site. For example, modification may, in some embodiments, comprise inducing expression of a specific cell-surface marker on the EVs of the invention, which results in specific interaction with a receptor on a desired target tissue. In one embodiment, the native contents of the EVs of the invention, such as EV100 or EV500, are removed and replaced with desired exogenous proteins or nucleic acids. In one embodiment, the native contents of EVs of the invention, such as EV100 or EV500, are supplemented with desired exogenous proteins or nucleic acids. In some embodiments, however, targeting of the EVs is not performed. In several embodiments, EVs of the invention, such as EV100 or EV500, are modified to express specific nucleic acids or proteins, which can be used, among other things, for targeting, purification, tracking, etc. In several embodiments, however, modification of the EVs of the invention is not performed. In some embodiments, the EVs of the invention, such as EV100 or EV500, do not comprise chimeric or recombinant molecules.

In some embodiments, subcutaneous or transcutaneous delivery methods are used. In some embodiments, nasal drops, nasal mist or aerosols or nasal gels or ointments are used. Due to the relatively small size, EVs of the invention, such as EV100 or EV500, are particularly advantageous for certain types of therapy because they can pass through blood vessels down to the size of the microvasculature, thereby allowing for significant penetration into a tissue. In some embodiments, this allows for delivery of the EVs of the invention, such as EV100 or EV500, directly to central portion of the damaged or diseased tissue (e.g., to the central portion of a tumor or an area of infarcted cardiac tissue). In addition, in several embodiments, use of EVs of the invention, such as EV100 or EV500, is particularly advantageous because the EVs of the invention can deliver their payload (e.g., the resident nucleic acids and/or proteins) across the blood brain barrier, which has historically presented an obstacle to many central nervous system therapies. In certain embodiments, however, EVs of the invention, such as EV100 or EV500, may be delivered to the central nervous system by injection through the blood brain barrier. In several embodiments, EVs of the invention, such as EV100 or EV500, are particularly beneficial for administration because they permit lower profile delivery devices for administration (e.g., smaller size catheters and/or needles). In several embodiments, the smaller size of EVs of the invention, such as EV100 or EV500, enables their navigation through smaller and/or more convoluted portions of the vasculature, which in turn allows EVs to be delivered to a greater portion of most target tissues.

In several embodiments, EVs of the invention, such as EV100 or EV500 derived from Muse sells are administered in combination with one or more additional agents. For example, in several embodiments, the EVs of the invention are administered in combination with one or more proteins or nucleic acids that are naturally comprised in EVs derived from Muse cells (e.g., to increase the concentration of said one or more protein or nucleic acid in the EVs derived from muse cells). In several embodiments, the Muse cells are administered in conjunction with the EVs of the invention, such as EV100 or EV500. In several embodiments, such an approach advantageously provides an acute and more prolonged therapeutic effect (e.g., immediate effect based on the actual EV administration and long term effect due to Muse cell delivery, wherein the Muse cells continue to secrete EVs post-administration).

In several embodiments, EVs of the invention, such as EV100 or EV500, are delivered in conjunction with a more traditional therapy, e.g., surgical therapy or pharmaceutical therapy. In several embodiments such combinations of approaches result in synergistic improvements in the viability and/or function of the target tissue. In some embodiments, EVs of the invention, such as EV100 or EV500, may be delivered in conjunction with a gene therapy vector (or vectors), nucleic acids (e.g., those used as siRNA or to accomplish RNA interference), and/or combinations of exosomes derived from other cell types.

The dose of the EVs, such as EV100 or EV500, or the dose of the pharmaceutical composition of the invention administered, depending on the embodiment, ranges from about 1.0×10⁵ to about 1.0×10⁹ EVs, including about 1.0×10⁵ to about 1.0×10⁶, about 1.0×10⁶ to about 1.0×10⁷, about 1.0×10⁸ to about 5.0×10⁸, about 5.0×10⁸ to about 1.0×10⁹, about 1.0×10⁹ to about 5.0×10⁹, about 5.0×10⁹ to about 1.0×10¹⁰, 1.0×10¹⁰ to about 5.0×10¹⁰, or higher, and overlapping ranges thereof. In certain embodiments, the dose is administered on a per kilogram basis, for example, about 1.0×10⁵ EVs/kg to about 1.0×10⁹ EVs/kg. In additional embodiments, EVs, such as EV100 or EV500, are delivered in an amount based on the mass of the target tissue, for example about 1.0×10⁵ EVs/gram of target tissue to about 1.0×10⁹ EVs/gram of target tissue. In several embodiments, EVs, such as EV100 or EV500, are administered based on a ratio of the number of EVs to the number of cells in a particular target tissue, for example EVs:target cell ratio ranging from about 10¹⁰:1 to about 1:1, including about 10¹⁰:1, about 10⁹:1, about 10⁸:1, about 10⁷:1, about 10⁶:1, about 10⁶:1, about 10⁴:1, about 10³:1, about 10²:1, about 10:1, and ratios in between these ratios. In additional embodiments, EVs, such as EV100 or EV500, are administered in an amount about 10-fold to an amount of about 1,000,000-fold greater than the number of cells in the target tissue, including about 50-fold, about 100-fold, about 500-fold, about 1000-fold, about 10,000-fold, about 100,000-fold, about 500,000-fold, about 750,000-fold, and amounts in between these amounts. If the EVs, such as EV100 or EV500, are to be administered in conjunction with the concurrent therapy, the dose of EVs, such as EV100 or EV500, administered can be adjusted accordingly (e.g., increased or decreased as needed to achieve the desired therapeutic effect).

In several embodiments, the EVs, such as EV100 or EV500, or the pharmaceutical composition of the invention, are delivered in a single, bolus dose. In some embodiments, however, multiple doses of EVs, such as EV100 or EV500, or the pharmaceutical composition of the invention, may be delivered. In certain embodiments, EVs, such as EV100 or EV500, or the pharmaceutical composition of the invention, can be infused (or otherwise delivered) at a specified rate over time. In several embodiments, when EVs, such as EV100 or EV500, or the pharmaceutical composition of the invention, are administered within a relatively short time frame after an adverse event (e.g., an injury or damaging event, or adverse physiological event such as an MI), their administration prevents the generation or progression of damage to a target tissue. For example, if EVs, such as EV100 or EV500, or the pharmaceutical composition of the invention, are administered within about 20 to about 30 minutes, within about 30 to about 40 minutes, within about 40 to about 50 minutes, within about 50 to about 60 minutes post-adverse event, the damage or adverse impact on a tissue is reduced (as compared to tissues that were not treated at such early time points). In some embodiments, the administration is as soon as possible after an adverse event. In some embodiments the administration is as soon as practicable after an adverse event (e.g., once a subject has been stabilized in other respects). In several embodiments, administration is within about 1 to about 2 hours, within about 2 to about 3 hours, within about 3 to about 4 hours, within about 4 to about 5 hours, within about 5 to about 6 hours, within about 6 to about 8 hours, within about 8 to about 10 hours, within about 10 to about 12 hours, and overlapping ranges thereof. Administration at time points that occur longer after an adverse event are effective at preventing damage to tissue, in certain additional embodiments.

For treatments of a systemic disorder or disease, the pharmaceutical composition can be administered systemically, e.g. via intravenous administration, or oral administration. In some embodiments, the pharmaceutical composition is administered locally, e.g. by injection into the site in need thereof (e.g. joints, skin, scalp), intravenous administration, intranasal infusion or sniffing (to boost neuroregeneration and cognition), intramuscular administration, or as an aerosol (e.g. to treat inflammation of the lung).

In one embodiment, the subject is an animal, preferably wherein the animal is a mammal, more preferably, wherein the mammal is a human.

The EVs (e.g. EV100, EV500 or combination thereof) or pharmaceutical composition of the invention comprising the EVs obtained from Muse cells or Muse cell-enriched MSC cultures, can be administered at a therapeutically or prophylactically-effective dose. In some embodiments, the pharmaceutical composition comprising the EVs obtained from Muse cells or Muse cell-enriched MSC cultures is administered once, twice or multiple times. In some embodiments, the pharmaceutical composition comprising the EVs obtained from Muse cells or Muse cell-enriched MSC cultures is administered repeatedly over a period of 1-12 months, e.g. 2 months, 3 months, 6 months, or more until the desired therapeutic effect is determined.

The following examples illustrate the invention, and are intended to be non-limiting embodiments of the invention.

### Specific Embodiments

In one embodiment, the invention provides exosomes for use in the treatment of migraine in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 10% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 20% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 30% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 40% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 50% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 60% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 70% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 10% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 20% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 30% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 40% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 50% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 60% Muse cells, and wherein the exosomes are administered in one or more doses of at least 10⁵ exosomes per dose.

In one embodiment, the invention provides exosomes for use in the treatment of migraine in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 70% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 10% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 20% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 30% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 40% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 50% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 60% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 70% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 10% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 20% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 30% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 40% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 50% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 60% Muse cells, and wherein the exosomes are administered in one or more doses of at least 10⁵ exosomes per dose.

In one embodiment, the invention provides exosomes for use in the treatment of rheumatoid arthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 70% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 10% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 20% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 30% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 40% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 50% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 60% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 70% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 10% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 20% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 30% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 40% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 50% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 60% Muse cells, and wherein the exosomes are administered in one or more doses of at least 10⁵ exosomes per dose.

In one embodiment, the invention provides exosomes for use in the treatment of Covid 19 in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 70% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 10% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 20% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 30% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 40% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 50% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 60% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 70% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 10% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 20% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 30% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 40% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 50% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 60% Muse cells, and wherein the exosomes are administered in one or more doses of at least 10⁵ exosomes per dose.

In one embodiment, the invention provides exosomes for use in the treatment of Rosacea in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 70% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 10% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 20% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 30% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 40% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 50% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 60% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 70% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 10% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 20% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 30% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 40% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 50% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 60% Muse cells, and wherein the exosomes are administered in one or more doses of at least 10⁵ exosomes per dose.

In one embodiment, the invention provides exosomes for use in the treatment of Melasma in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 70% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 10% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 20% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 30% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 40% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 50% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 60% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC cultures comprise at least 70% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 10% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 20% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 30% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 40% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 50% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 60% Muse cells, and wherein the exosomes are administered in one or more doses of at least 10⁵ exosomes per dose.

In one embodiment, the invention provides exosomes for use in the treatment of Diabetic neuropathy in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC cultures comprise at least 70% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 10% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 20% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 30% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 40% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 50% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 60% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 70% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 10% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 20% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 30% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 40% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 50% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 60% Muse cells, and wherein the exosomes are administered in one or more doses of at least 10⁵ exosomes per dose.

In one embodiment, the invention provides exosomes for use in the treatment of Osteoarthritis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 70% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 10% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 20% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 30% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 40% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 50% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 60% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 70% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 10% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 20% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 30% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 40% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 50% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 60% Muse cells, and wherein the exosomes are administered in one or more doses of at least 10⁵ exosomes per dose.

In one embodiment, the invention provides exosomes for use in the treatment of Psoriasis in a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 70% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 10% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 20% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 30% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 40% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 50% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 60% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell MSC cultures, wherein the high Muse cell MSC cultures are derived from umbilical cord MSC, wherein the high Muse cell MSC comprise at least 70% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 10% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 20% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 30% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 40% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 50% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 60% Muse cells, and wherein the exosomes are administered in one or more doses of at least 10⁵ exosomes per dose.

In one embodiment, the invention provides non-therapeutic use of the exosomes of the invention for preventing hair loss or promoting hair growth in a subject; and/or for improving the appearance of wrinkles of the skin of a subject, wherein the exosomes are below 220 μm in diameter, wherein the exosomes are derived from high Muse cell SC cultures, wherein the high Muse cell SC cultures are derived from amniotic epithelial SC, wherein the high Muse cell amniotic epithelial SC comprise at least 70% Muse cells, and wherein the exosomes are administered in one or more doses of at least 10⁵ exosomes per dose.

Alternatively, the exosomes of the invention for use in treatment are above 220 μm in diameter.

### Examples

### Materials and Methods

### M1 Medium (Isolation Medium):

M1 culture medium is used to plate and support the initial proliferation of Muse cells in the single cell suspension obtained from the tissue comprising Muse cells. M1 culture medium consists of mammalian cell adjusted basal media (such as DMEM, RPMI or other), which has been supplemented with fetal calf serum, fetal bovine serum, human platelet lysate, insulin, or transferrin at a concentration of 1-25%.

An exemplary M1 medium comprises DMEM with 1 g/L glucose, sodium pyruvate 110 mg/L, stable glutamine 200 mM±50, human platelet lysate (cryoprecipitate) 1-7.5%, fetal calf serum 1-10%, insulin 10-100 ng/L, transferrin 10-200 ng/L.

Alternatively, M1 culture medium consist of mammalian cell adjusted basal media (such as DMEM, RPMI or other) supplemented with human cord blood plasma, human platelet lysate, insulin, or transferrin at a concentration of 1-25%.

Hence, another exemplary M1 medium comprises DMEM with 1 g/L glucose, sodium pyruvate 110 mg/L, stable glutamine 200 mM±50, human platelet lysate (cryoprecipitate) 5%, human cord blood plasma 5%, insulin 10-100 ng/L, transferrin 10-200 ng/L.

### M2 Medium (Expansion Medium):

M2 proliferation medium supports the growth and proliferation of Muse cells in the culture and is used throughout the passaging and expansion of Muse cells. It contains mammalian cell adjusted basal medium (such as DMEM, RPMI or other), supplemented with the following additives: human cord blood plasma, human platelet lysate, fibroblast growth factor, stem cell factor, VEGF, transferrin, selenium, insulin, human growth hormone, nerve growth factor, vitamin C, D, E, A, and valproic acid.

An exemplary M2 medium comprises DMEM 1-5 g/L glucose, 1-10% human cord blood plasma, 10-100 IU/ml heparin, 2-5% human PRP lysate, fibroblast growth factor 10-50 ng/L, stem cell factor 5-20 ng/L, VEGF 2-20 ng/L, transferrin 10-100 ng/L, selenium 50-200 ng/L, insulin 10-200 ng/L, human growth hormone 0.2-1 IU/L, nerve growth factor 50-200 ng/L, vitamin C, D, E, A, 0.2-5% and valproic acid 0.2-4%.

### M3 Medium (EV Production Medium):

M3 medium provides for the increased secretion of extracellular vesicles (EVs), including exosomes and microvesicles. An exemplary M3 medium comprises M3 phenol free media, DMEM or RMPI with a maximum of 5% of cord blood plasma or human platelet lysate.

Surface matters: for proliferation and support of Muse cells, we need to use appropriate surface coatings. These can include collagen, poly-Lysine, gelatin, Matrigel, either used alone or in combination.

### Buffer for Extracellular Vesicle (EV) Storage and Lyophilization:

An exemplary buffer for EV storage and lyophilization comprises:

| | |
|---|---|
| NaH₂PO₄ | 1.2 g/l |
| Glycine | 1.05 g/l |
| Mannitol | 27.32 g/l |
| Tween-20 | 2.5 g/l |
| Trehalose | 50 mM |

Another example of buffer for EV storage and lyophilization comprises:

| | |
|---|---|
| NaH₂PO₄ | 1.38 g/l |
| Glycine | 1.05 g/l |
| Mannitol | 27.24 g/l |
| Tween-20 | 2.5 g/l |
| Trehalose | 50 mM |

### Coating of Cell Culture Surfaces

Muse cell cultures may be cultured in coated culture dishes or culture flasks. Cell culture flask were coated with collagen, poly-lysine, gelatin, Matrigel, or a combination of the same prior to culturing the Muse cells.

### Examples

Examples provided below are intended to be non-limiting embodiments of the invention.

### Example 1: Obtaining Muse Cells

Muse cells are a subpopulation of stem cell-like cells that are present in very low quantities in various tissues, such as adipose tissue, bone marrow, umbilical cord, placenta. The present example demonstrates one exemplary way of obtaining Muse cells from tissue. Any tissue was obtained with written consent from the donors. For umbilical cord blood or umbilical cord tissue, as well as placenta, the tissue samples were obtained from healthy, voluntary donors and/or healthy and full-term births.

In order to obtain Muse cells, a single cell suspension was prepared from the tissue comprising Muse cells. Tissue donors are screened for infectious disease according to EU requirements for tissue donations.

To obtain muse cells from solid tissue, tissue was washed a minimum of three times in sterile PBS. With sterile forceps and scissors, tissue was cut to reveal vessel (mark vein or artery) or appropriate tissue part for cell isolation in the case of the amnion. Tissue of interest (such as blood vessel, amniotic membrane layer, or Wharton's jelly layer) was cut to up to 4 mm width and minced. Tissues were mixed with collagenase buffer containing 0.1% collagenase and calcium gluconate 0.01%, in RPMI or DMEM media, and incubated for 1-2 hours at 37° C. or overnight at 4° C.-8° C. Following incubation, debris should be completely digested. Any remaining debris was removed by straining the cell solution with an equal volume RMPI medium through a 100 μm cell strainer. Filtered solution was centrifuged at 600-800 g for 5-7 min and sedimented cells were resuspended in M1 Medium. In order to proliferate Muse cells in culture, the single cell suspension comprising Muse cells was seeded onto coated culture dishes and cultured in a humidified atmosphere with 5% carbon dioxide with M1 culture medium (seeding medium) until the cells reached about 80-90% confluency. All cell culture steps were performed either 2D (on coated culture dishes) or 3D bioreactors (collagen-coated or glass or cytodex beads as carriers).

A general overview of the inventive method is provided in FIGS. 1 and 2. The inventive method generally relies on culturing the cells in a step-wise manner, wherein after initial plating in a collection medium (M1), the cells are cultured and expanded in the first culture medium (M2), and the production of EVs is induced by the second culture medium (M3). After harvesting the EVs, the cells that are enriched in Muse cells are further replated in M2, to further expand the cells. This process of expansion and harvesting maintains the Muse cells in an undifferentiated state and maximizes the yield of Muse cells and Muse-cell derived EVs.

An exemplary timeline of the method steps is indicated in the arrow in both figures, which is not meant to be limiting, but can vary by one to three days for each step. Muse cells from whole blood, plasma, bone marrow or serum were obtained by using density gradient centrifugation as previously described [10].

For Muse cell enrichment from bone marrow, bone marrow was collected from consenting adults into K2 EDTA tubes. Following centrifugation at 450 g for 10 minutes, the buffy coat was isolated and layered onto Ficoll, for density gradient centrifugation at 400 g for 20 minutes. The cells at the interface were washed in PBS and plated on coated flasks in M1 medium.

### Example 2: Culturing of Muse Cells

Muse cells are present at very low percentages, typically below 1-2%, in their native tissues (Table 1).

TABLE 1

| | Relative |
|---|---|
| | quantity of |
| | Muse cells |
| | in tissues |
| Tissue source | [%] |
| Placenta | 0.1-2 |
| Umbilical cord | 0.1-1 |
| Bone marrow | 0.1-0.5 |
| Adipose tissue | 0.1-2 |
| Skin grafts | 0.1-0.5 |
| Decidual teeth | 0.1-2 |

In order to proliferate Muse cells in culture, the single cell suspension comprising Muse cells was seeded onto coated culture dishes and cultured in a humidified atmosphere with 5% carbon dioxide with M1 culture medium (seeding medium) until the cells reached about 80-90% confluency. All cell culture steps were performed either 2D (on coated culture dishes) or 3D bioreactors (collagen-coated or glass or cytodex beads as carriers).

After 80-90% confluency was reached, the cells were passaged by re-plating at a lower density using M2 medium (expansion medium). The cultivation in M1 and M2 allows to propagate Muse cells in culture and to expand their quantity multi-fold, while maintaining the native and regenerative status of Muse cells, which can be verified by routine methods, e.g. FACS (fluorescence-activated cell sorting) for SSEA3+, CD105+. The relative quantity of Muse cells in the culture, as well as their native and regenerative status, can be determined by the co-expression of SSEA3 and CD105 by conventional methods, such as FACS or MACS (magnetic-activated cell sorting). During culturing and prior to harvesting the extracellular vesicles, the relative quantity of Muse cells in the cell culture dish was determined.

Cell cultures that comprised less than 10% Muse cells were discontinued.

The Muse cells were cultured in M2 medium until a confluency of about 70% was reached and then passaged, i.e. split into multiple new pre-coated flasks, in order to maintain the regenerative and undifferentiated Muse cell characteristics (SSEA3+, CD105+). In some embodiments, the cells were passaged when the confluency was less about 60-70%, or less than 60%.

The Muse cells can be continuously cultured and passaged up to P8 in M2 medium. Muse cells exist in mixed cultures and continue to proliferate. The Muse cell phenotype was observed by FACS for SSEA3+. Cells were cultivated up to P8 as later passages were associated with less regenerative potential.

As shown in FIG. 12, the proportion of SSEA3+ positive cells, i.e. Muse cells, decreases with increasing number of passages.

After an average of four passages (P4), the Muse cell cultures were seeded into flasks in M2 medium at a density of 2,000-6,000 cells per cm² (2D culture), or seeded with suitable 3D carriers or scaffolds at a concentration of 100-5000 cells per 1 mg of carrier (3D culture; e.g. a collagen carrier).

Once cells were attached and started proliferating (cells divide every 14-18 hours), the M2 medium was replaced with M3 medium (EV production medium). Cells were cultured in M3 medium until they reached a maximum of about 80-90% confluency. Once about 80-90% confluency was reached, the extracellular vesicles (EVs) were harvested.

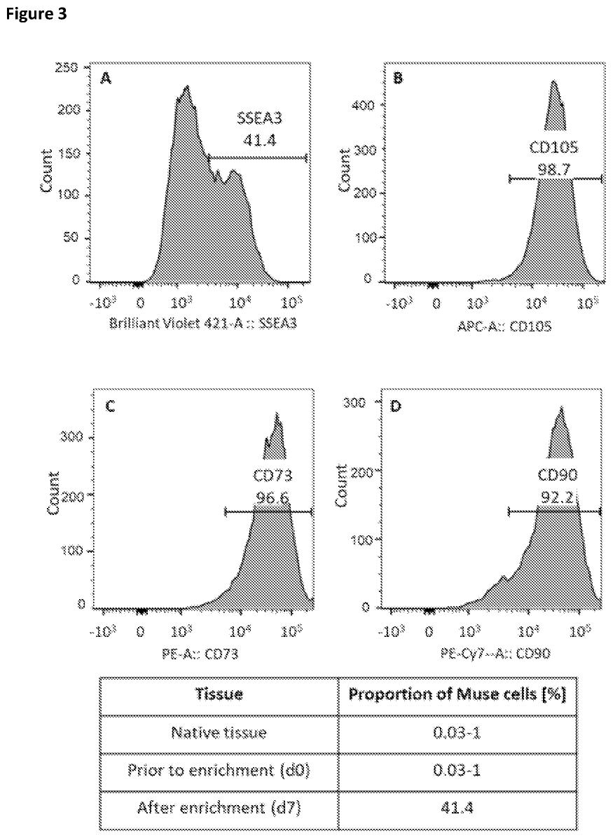

Using this culturing technique, we were able to significantly increase the concentration of Muse cells (FIG. 3).

FIG. 3 shows the enrichment of Muse cells from umbilical cord. The Muse cell concentration in umbilical is typically between 0.01-1%, but was enriched using the method of the invention to over 40% (FIG. 3). The EVs collected from this cell culture is thus also enriched for Muse cell derived EVs, which was not previously possible using any method of the art.

The EVs obtained by the method provided herein are produced by robust, pluripotent cells.

### Example 3: FACS Sorting of Muse Cells

Presence and relative quantity of Muse cells can be determined by FACS or MACS. The cells were stained with antibodies (all from Becton Dickinson) specific for surface markers SSEA-3 (Brilliant Violet 421-A), CD105 (APC-A), CD73 (PE-A), and CD90 (PE-Cy7-A). Briefly, Muse cells were suspended in PBS and incubated with the combination of monoclonal antibodies described above. Unstained cells were included as a control. Cells were processed through an LSR II FACS (Becton Dickinson) and BD FACSDiva Software.

Muse cells were identified as being SSEA-3+ and CD105+. It shall be appreciated that in several embodiments, EVs may be obtained from other cell types than exemplified herein. In some exemplary embodiments, the Muse cells are identified by the presence of at least two markers selected from the group consisting of SSEA3, CD105, CD90, and CD73. In yet another embodiment, the Muse cells are identified to express at least one of NANOG, Oct-4, Sox-2, Klf4, or c-Myc. In another embodiment, the absence of CD45 was confirmed. In yet another embodiment, the presence of Muse cells was confirmed by identification of cells that were SSEA3+, CD105+, CD90+, CD45−. In some embodiments, the presence of Muse cells was confirmed by identification of cells that were SSEA3+, CD105+, and CD90+.

An exemplary enrichment of Muse cells is shown in FIG. 3, which shows the FACS analysis of high muse cell content MSC culture from human umbilical cord tissue that was obtained from a full-term, healthy birth. The pluripotent marker SSEA3+ reveals 41.4% Muse cells in the population. 96.6%, 92.2%, 98.7% of the cells were positive for CD73, CD90, and CD105, respectively. These results indicate that the culture comprises almost exclusively Muse cells and other MSCs, wherein the culture is highly enriched for Muse cells.

Further exemplary enrichments are provided in Table 2. Tissue sources for Muse cell cultures include placental AESC mixed Muse cell culture, umbilical cord Wharton's jelly high Muse MSC culture, adult bone marrow MSC cultures (Donors no. 1-3), adult adipose MSC cultures (Donors no. 4-6). Adult donors were an average age of 48±5 years old, with equal numbers of male and female donors. Cells were collected and were stained with monoclonal antibodies for CD73, CD90, CD105, and SSEA-3 in routine manner. The percentage of cells from each culture positive for each marker is provided in Table 2. The FACS plots for sample labelled “Umbilical cord no.1” are shown in FIG. 3.

TABLE 2

| Donor | CD73 | CD90 | CD105 | SSEA3+ |
|---|---|---|---|---|
| Umbilical cord no. 1 | 96.6 | 92.2 | 98.7 | 41.4 |
| Placenta | 98.9 | 99.4 | 66.9 | 34.1 |
| Umbilical cord no.2 | 95.6 | 99.7 | 77.6 | 25.2 |
| (incl. Wharton's jelly) | | | | |
| MSC no. 1 | 96.7 | 98.4 | 89.6 | 2.3 |
| MSC no. 2 | 99.4 | 99.2 | 83.1 | 4.9 |
| MSC no. 3 | 96.9 | 98.6 | 62.1 | 1.9 |
| MSC no. 4 | 97.3 | 99.7 | 62.7 | 10.2 |
| MSC no. 5 | 98.6 | 98.7 | 86.4 | 4.3 |
| MSC no. 6 | 99.3 | 100 | 61 | 2.6 |

The results demonstrate that the method provided herein could enrich Muse cells from 0.1-2% starting concentrations in placenta and umbilical cord to 41.4%, 34.1%, and 25.2%, respectively. Muse cells in adult mesenchymal tissues could also be enriched, however, not as efficiently as from placenta and umbilical cord tissues. This suggests that Muse cells from tissues derived from perinatal sources are more robust and better able to proliferate under stress.

### Example 4: Obtaining Extracellular Vesicles (EVs)

After the Muse cell culture in M3 medium reached a suitable confluency, e.g. about 80-90%, collection or harvesting of extracellular vesicles (EVs) commenced.

Collection of the EV-enriched culture medium (“harvesting”), was performed repeatedly, e.g. every 18-36 hours by collecting the entire M3 culture medium, and feeding the cells with fresh M3 medium. Muse cells in M3 medium were not further passaged, since Muse cells grow slowly in M3 medium, but produce EVs very efficiently and robustly.

The harvesting was repeated for up to three times. Subsequently, the Muse cells were trypsinized and re-plated at lower density in M2. Once the cells reached again about 80-90% confluency, the M2 is replaced with M3 and the EV-enriched medium was harvested again.

The cycle of repeatedly harvesting the EV-enriched medium, and re-plating was repeated for up to eight to nine passages (P8-P9). After the EV-enriched medium of P9 cell culture was obtained, the cells were discarded.

The harvested EV-enriched medium was stored for up to 96 hours at 4-8° C. and processed further for EV isolation.

This procedure allows for the isolation of EVs from multiple passages of high muse cell MSC cultures obtained from multiple MSC sources while maintaining muse cell pluripotency.

### Example 5: Culturing of Muse Cells in Bioreactor

### Expansion in M2

Cells were initially seeded in flasks and cultured in M1 medium as described above. 750 ml of pre-warmed cell expansion medium (M2) was mixed with sterile, pre-hydrated and L-Lys or Collagen pre-coated microcarriers (55 g at 360 cm²/g) and 10⁸ mesenchymal stem cells. Mixture was immediately inoculated into a 2-liter bioreactor. Additional 250 ml of M2 was pumped through the same tubes as the mixture to fully wash the tubes of cells and microcarriers. The bioreactor was subjected to 50% dissolved oxygen (DO), 37° C. and pH 7.4. At day 1, the agitation is configured in intervals, 10 min without agitation and 150 min with 100 rpm agitation.

### Exosome Production in M3

At day 2, one liter of M2 medium was exchanged for EV production medium (M3) and the agitation was changed from intervals to continuous agitation at 100 rpm. At days 5, 6 and 7, half of the medium (now conditioned medium) was changed to fresh medium and the conditioned medium was collected. Conditioned medium was filtered through 0.2 um pore filter and stored at 4° C. until exosome extraction.

### Optional Cell Harvest and Storage

At day 8 all medium with microcarrier/cells was collected in a sterile bottle and left for 3-5 min to settle. 1.5 liter of medium was taken out and 1.5 liter of PBS was added. The bottle was left for 3-5 min for microcarrier/cells to settle. The process was repeated 2 times. Afterwards, prewarmed TrypLE was added to the bottle, and the bottle was placed in 37° C. incubator with agitation for 15 minutes. Following the 15-minute incubation, a small sample was collected to verify that the cells were detaching from the microcarriers. The slurry was passed through a 100 μm pore strainer. Harvested cells in a final volume of approximately 2 L were centrifuged at 450×g for 5 minutes at 4° C. twice. Cell pellets were resuspended at 20×10⁶ viable cells/mL in CryoSure DEX/Cord blood plasma mixture and aliquoted into glass vials at 5 mL per vial. Vials were transferred to a freezing container and placed at −80° C. for at least 4 hours followed by storage in the vapor phase of liquid nitrogen.

### Example 6: Quality Control

Quality Control of the Muse cells was performed before and after EV production. Tissue donors were screened for infectious disease according to EU requirements for tissue donations. Prior to transferring initial cultures into M2 Medium, nucleic acid amplification testing (NAT) was carried out to rule out viruses/pathogens and FACS analysis was performed to assess the expression of markers, e.g., the simultaneous expression or co-expression of two, three, or more markers selected from the group consisting of SSEA3, CD105, CD90, CD73, NANOG, Oct-4, Sox-2, Klf4, and c-Myc. Further, in some embodiments, the absence of CD45 was confirmed. For example, the presence of Muse cells was confirmed by expression of cells that were SSEA3+, CD105+, CD90+, CD45−. Prior to separating EV100s from EV500s, any cellular debris in the collected M3 medium containing the EVs was removed by spinning the medium at 3000-4500 g for 10 min at 4C.

These multiple quality control steps ensure that a high Muse cell culture is expanded, pathogen free, and that a clean, uncontaminated EV preparation from a high Muse cell culture is consistently produced.

### Example 7: Separating Exosomes (EV100) and Microvesicles (EV500)

The harvested M3 medium comprises the entire secretome, which is thought to have regenerative properties, but comprises secreted proteins in addition to the vesicular fraction.

In order to assess the regenerative and therapeutic function of the smaller and larger vesicular fractions, i.e. the exosome (EV100) and microvesicles (EV500), both fractions were separated.

(1) In order to separate the exosomes (EV100) from the microvesicles (EV500), the M3 medium was first spun at 3000-4500 g for 10 min, 4° C. to remove cellular debris. The resulting supernatant is considered the secretome, which contains the EV particles, exomeres, and soluble proteins. The total secretome was then centrifuged at 12,000 g for 25-30 minutes at 4° C. The pellet contained the microvesicles (EV500), and the supernatant comprised the remaining fractions of the secretome, including the exosome fraction (EV100).

The supernatant containing the exosomes (EV100) was further subjected to tangential filtration using a filtration membrane of 150-300±50 kDa pore size to obtain exosomes (EV100) having a diameter of 220 nm or less, with an mean diameter of about 150 nm.

(2) Alternatively to the centrifugation based method above, the harvested M3 medium of Example 4 was subjected to gel filtration to separate the hydrophobic particles with a size of 220-500 nm. To further separate the EV100s from the remaining secretome components, the EV100 fraction was processed through gel filtration. The final EV100 fraction was filtered through a 220 nm membrane with the final fraction having a mean diameter of about 100 nm.

These steps resulted in the isolation of conditioned medium/secretomes from high Muse cell content MSCs and the separation of EV100 and EV500 particles from the secretome. In addition, these steps prepared them for storage through freezing in suspension, or lyophilization according to Example 9 below.

### Example 8: Exosome Purification Using Gel Filtration Chromatography

Harvested M3 medium from bioreactor production was subjected to tangential flow filtration using a VivaFlow 200 filter with a 100-kDa molecular weight cutoff (MWCO) and concentrated 10-fold (volume reduced 10-fold). In the next step the cell culture medium was exchanged with sodium chloride saline, by continuously feeding the system with sodium chloride to replace the loss of permeate.

The filtered conditioned medium from the bioreactor was further purified by gel filtration chromatography after tangential flow filtration. A BPG 140/500 column loaded with 5 liters of CL-6B sorbent was used. First, the column was calibrated using the storage buffer (lyophilization buffer comprising 1.38 g/l NaH2PO4, 1.05 g/l Glycine, 27.24 g/l Mannitol, 2.5 g/l Tween-20, 50 mM Trehalose), with two column volumes at a speed of 80 ml/min, without exceeding a pressure of 0.4 Bar. After calibration, the sample was loaded onto the column at 1/25 of the working column volume, i.e. 200 mL, and an isocratic gradient was run. During the isocratic gradient, the first UV peak fraction was collected. The first UV peak was the exosome fraction, since in gel filtration the particles are separated by size and exosomes are the largest particle. Exosome and protein peaks may overlap, therefore, collection was stopped when the exosome peak subsided and there was at least a small rise in UV, in order to avoid collecting the protein fraction. The column can be reused once it has been washed out with storage buffer.

D-trehalose was added to a final concentration of 50 mM to the collected fraction, and the product was sterile filtered using a 200-nm filter, aliquoted, frozen and/or freeze-dried.

### Example 9: Quantification of EVs, EV100 or EV500

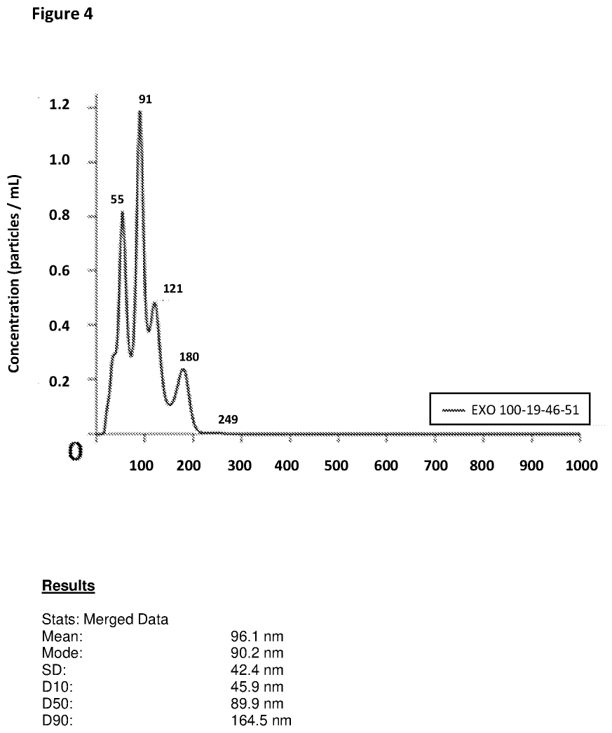

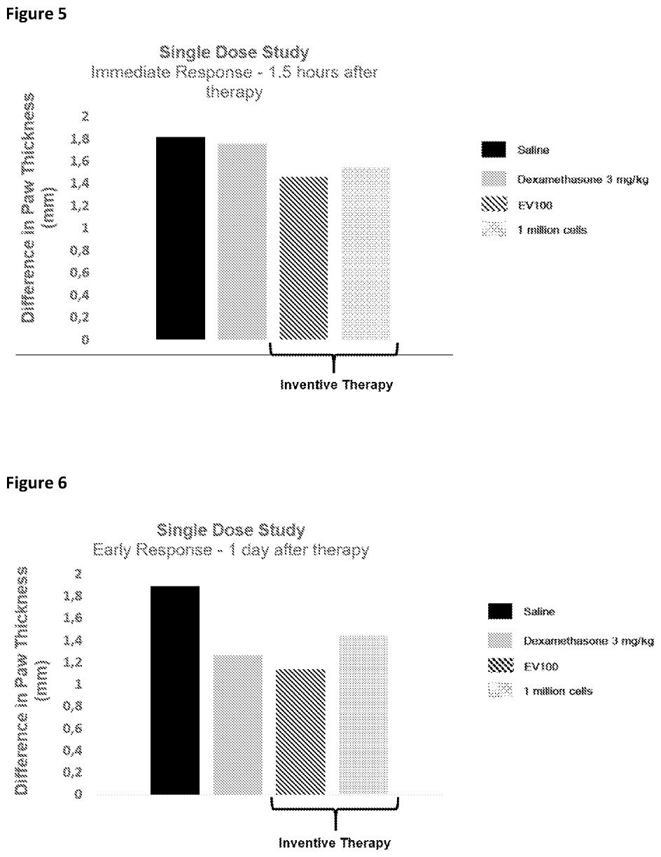

EV, EV100 and EV500 fractions obtained as described above were quantified using the Nanosight NS300 (Malvem Analytical) according to the Nanosight manual. An exemplary quantification is presented in FIG. 4, which shows a purified EV100 particle preparation after lyophilization. The sample was resuspended in PBS before quantification. The results show a concentration for EV100s of 6.68×10⁸ particles/mL. The size distribution is described in Example 8 below.

Optionally, the EVs, EV100 or EV500 fractions were concentrated and/or adjusted to the same concentrations of nucleic acids or proteins.

The results show that a high concentration of EV100 particles can be obtained from fractionation of the conditioned medium of a high Muse cell MSC culture. Further processing to lyophilize the particles would allow the concentration to be adjusted based on the volume of solute added to the dried particles.

### Example 10: Determining Size and Content of Extracellular Vesicles

The size distribution of the EVs was determined using a Nanosight NS300 (Malvem Analytical) according to the description in Example 7. EV100 and EV500 samples were measured after lyophilization and resuspension in PBS. The concentration of nucleic acids was determined using a Qubit fluorometer (Fisher Scientific) according to the manufacturer's instructions. Protein concentrations were evaluated using the Bradford Assay (Fisher Scientific) according to manufacturer's instructions. Lyophilization buffer was used as a blank/control.

The Muse cell derived exosome fraction (EV100) has a highly regenerative potential and comprises extracellular vesicles having a diameter of about 220 nm or less, such as 220 nm, 200 nm, 180 nm or less. In some embodiments, the EV100 fraction comprises EVs having a diameter of about 30 to 220 nm.

The Muse cell derived microvesicle fraction (EV500) also has regenerative potential and comprises extracellular vesicles having a diameter of between 220 to about 550 nm, such as 230-550 nm, 250-500 nm, 300-450 nm. In some embodiments, the EV500 fraction comprises EVs having a diameter of about 450-550 nm.

In one exemplary embodiment, the size distribution of the EV100 and EV500 fractions are as shown in Table 3:

TABLE 3
Extracellular vesicle parameter

| Parameter | EV100 | EV500 |
|---|---|---|
| Nucleic acid concentration | 0.01-0.8 | 0.05-1.8 |
| (ng/ul) | | |
| Protein concentration (μg/ml) | 18-40 | 50-180 |
| Diameter (nm) | 148 ± 70 | 490 ± 60 |
| Endotoxin IU/ml | <2 | <2 |

| | |
|---|---|
| Sterility | sterile |

The nucleic acid concentration and protein concentration in EV100 and EV500 fractions can differ (Table 3), which could be due to a number of factors. First, because EV100 and EV500 particles are produced through different pathways in the cell, it is possible that their endogenous (inherent) nucleotide and protein content differs by design. For example, preliminary characterization of EV100 and EV500 samples indicates that the miRNA content differs between the two particle types (Example 9, Table 3). In one study, in EV samples that were obtained from equivalent prostate cell numbers, protein concentration of a number of individual proteins was shown to differ between exosomes (EV100s) and microvesicles (EV500s) [11]. This suggests that the particles are not only unique in their size, but also in their contents and that their therapeutic potential will be due to common properties shared between EV100 and EV500, and some components that are unique to each particle type.

Both EV100 and EV500 were free of endotoxins and pathogenic contaminants. These data reveal that EV100 and EV500 particles differ not only in size, but also in their molecular and biochemical compositions.

### Example 11: Sterilization and Storage

The EV500 preparation comprising quantified EV500, which had previously undergone quality control, was frozen in storage buffer at a temperature below −20° C.

The EV100 preparation comprising quantified EV100 that had undergone quality control, was subjected to filtration to remove any larger particles using a 0.45 μm filter, and a secondary filtration step using a 0.22 μm filter. The filter-sterilized EV100 preparation was mixed with sterile storage buffer and frozen immediately at temperatures below −20° C. Long term storage of any type of liquid EVs was at temperatures below −80° C., wherein the EV preparations were stable for up to 6 months

For long term storage, EV100 or EV500 preparations were lyophilized. Known concentrations of EVs were aseptically filled into vials and placed at −80° C. for 12-24 hours. After freezing, the vials were placed into a lyophilization machine and freeze dried according to the protocol shown in Table 4 below.

TABLE 4

| | Lyophilization Parameters | | |
|---|---|---|---|
| | Time, | Preassure, | Temperature, |
| Stages | h | mBar | ° C. |
| Freezing | 0.5 | — | Up to −80 |
| Main cycle (cool + vacuum) | 12 | 0.3 | from −80 to 0 |
| Additional cycle | 6 | 0.3 | from 0 to 5 |
| (heat shelves) | | | |
| Additional cycle | 4 | 0.3 | from 5 to 10 |
| (heat shelves) | | | |

After lyophilization cycles were completed, stoppering of vials was performed under vacuum, and stored at −20° C. or below. The lyophilized EV preparations were stable for up to 2 years. Optionally, quality control of the lyophilized EV preparations was performed.

Storage of EVs in suspension indefinitely requires freezing at −80° C. They can be stored for up to one year at −20° C.; and only approximately one week at 4° C. Shipping samples in suspension may also require dry ice during transport to preserve the particles, which can be costly and restrictive. Lyophilization, on the other hand, provides a product that is more stable at ambient temperatures for longer periods and can be more conveniently shipped across any climate without the need for dry ice.

### Example 12: Microarray

For miRNA sequencing, a QIAseq kit was used to generate a miRNA library and sequencing was carried out using the Qiagen Illumina NGS System according to manufacturer's instructions. miRNAs detected in EV100 and EV500 of Muse cells are shown in Table 5.

TABLE 5

| | | | | |
|---|---|---|---|---|
| hsa-let-7a-2-3p | hsa-miR-1178-5p | hsa-miR-1244 | hsa-miR-1275 | hsa-miR-130b-5p |
| hsa-let-7a-3p | hsa-miR-1179 | hsa-mIR-1245b-3p | hsa-miR-1276 | hsa-miR-132-3p |
| hsa-let-7a-5p | hsa-miR-1180-3p | hsa-miR-1246 | hsa-miR-1277-5p | hsa-miR-132-5p |
| hsa-let-7b-3p | hsa-miR-1180-5p | hsa-miR-1247-3p | hsa-miR-128-1-5p | hsa-miR-1322 |
| hsa-let-7b-5p | hsa-miR-1181 | hsa-miR-1247-5p | hsa-miR-128-2-5g | hsa-miR-133a-3p |
| hsa-let-7c-3p | hsa-miR-1182 | hsa-miR-1249-3p | hsa-miR-128-3p | hsa-miR-133a-5p |
| hsa-let-7c-5p | hsa-miR-1183 | hsa-miR-1250-3p | hsa-miR-1281 | hsa-miR-133b |
| hsa-let-7d-3p | hsa-miR-1185-1-3p | hsa-miR-1251-3p | hsa-miR-1283 | hsa-miR-134-3p |
| hsa-let-7d-5p | hsa-miR-1185-2-3p | hsa-miR-1251-5p | hsa-miR-1284 | hsa-miR-134-5p |
| hsa-let-7e-3p | hsa-miR-1185-5p | hsa-miR-1252-5p | hsa-miR-1285-3p | bsa-miR-1343-3p |
| hsa-let-7e-5p | hsa-miR-1193 | hsa-moiR-1253 | hsa-miR-1286 | hsa-miR-135a-5p |
| hsa-let-7f-2-3p | hsa-miR-1197 | hsa-miR-1255b-5p | hsa-miR-1287-3p | hsa-miR-135b-5p |
| hsa-let-7f-5p | hsa-miR-1199-3p | hsa-miR-1257 | hsa-miR-1287-5p | hsa-miR-136-3p |
| hsa-let-7g-3p | hsa-miR-1199-5p | hsa-miR-125a-3p | hsa-miR-129-5p | hsa-miR-136-5p |
| hsa-let-7g-5p | hsa-miR-1200 | hsa-miR-125a-5p | hsa-miR-1290 | hsa-miR-137 |
| hsa-let-71-3p | hsa-miR-1202 | hsa-miR-125b-1-3p | hsa-miR-1291 | hsa-miR-138-1-3p |
| hsa-let-7i-5p | hsa-miR-1203 | hsa-miR-125b-2-3p | hsa-miR-1292-3p | hsa-miR-138-2-3p |
| hsa-miR-1-3p | hsa-miR-1207-3p | hsa-miR-125b-5p | hsa-miR-1292-5p | hsa-miR-138-5p |
| hsa-miR-1-5p | hsa-miR-1207-5p | hsa-miR-126-3p | hsa-miR-1293 | hsa-miR-139-3p |
| hsa-miR-100-3p | hsa-miR-122-5p | hsa-miR-126-5p | hsa-miR-1294 | hsa-miR-139-5p |
| hsa-miR-100-5p | hsa-miR-1224-3p | hsa-miR-1260a | hsa-miR-1295a | hsa-miR-140-3p |
| hsa-miR-101-3p | hsa-miR-1224-5p | hsa-miR-1260b | hsa-miR-1296-3p | hsa-miR-140-5p |
| hsa-miR-101-5p | hsa-miR-1225-3p | hsa-miR-1262 | hsa-miR-1296-5p | hsa-miR-141-3p |
| hsa-miR-103a-2-5p | hsa-miR-1226-5p | hsa-miR-1265 | hse-miR-1298-3p | hsa-miR-141-5p |
| hsa-miR-103a-3p | hsa-miR-1227-3p | hsa-miR-1266-5p | hsa-miR-1298-5p | hsa-miR-142-3p |
| hsa-miR-103b | hsa-miR-1229-3p | hsa-miR-1268a | hsa-miR-1299 | hsa-miR-142-5p |
| hsa-miR-105-3p | hsa-miR-1229-5p | hsa-miR-1268b | hsa-miR-1301-3p | hsa-miR-143-3p |
| hsa-miR-105-5p | hsa-miR-1231 | hsa-miR-1269a | hsa-miR-1303 | hsa-miR-143-5p |
| hsa-miR-106a-3p | hsa-miR-1233-3p | hsa-miR-127-3p | hsa-miR-1304-3p | hsa-miR-144-3p |
| hsa-miR-1063-5p | hsa-miR-1233-5p | hsa-miR-127-5p | hsa-miR-1304-5p | hsa-miR-144-5p |
| hsa-miR-106b-3p | hsa-miR-1234-3p | hsa-miR-1270 | hsa-miR-1305 | hsa-miR-145-3p |
| hsa-miR-106b-5p | hsa-miR-1236-3p | hsa-miR-1271-5p | hsa-miR-1306-3p | hsa-miR-145-5p |
| hsa-miR-107 | hsa-miR-1236-5p | hsa-miR-1272 | hsa-miR-1306-5p | hsa-miR-1469 |
| hsa-miR-10a-3p | hsa-miR-1237-3p | hsa-miR-1273c | hsa-miR-1307-3p | hsa-miR-146a-3p |
| hsa-miR-103-5p | hsa-miR-1237-5p | hsa-miR-1273e | hsa-miR-1307-5p | hsa-miR-146a-5p |
| hsa-miR-10b-3p | hsa-miR-1238-5p | hsa-miR-1273g-3p | hsa-miR-130a-3p | hsa-miR-146b-3p |
| hsa-miR-10b-5p | hsa-miR-124-5p | hsa-miR-1273g-5p | hsa-miR-130a-5p | hsa-miR-146b-5p |
| hsa-miR-1178-3p | hsa-miR-1243 | hsa-miR-1273h-3p | hsa-miR-130b-3p | hsa-miR-148a-3p |
| hsa-miR-148a-5p | hsa-miR-186-5p | hsa-miR-199a-3p | hsa-miR-215-5p | hsa-miR-26b-3p |
| hsa-miR-148b-3p | hsa-miR-187-3p | hsa-miR-199a-5p | hsa-miR-216a-3p | hsa-miR-26b-5p |
| hsa-miR-148b-5p | hsa-miR-187-5p | hsa-mil-199b-3p | hsa-miR-216a-5p | hsa-miR-27a-3p |
| hsa-miR-149-3p | hsa-miR-188-3p | hsa-miR-199b-5p | hsa-miR-216b-3p | hsa-miR-27a-5p |
| hsa-miR-149-5p | hsa-miR-188-5p | hsa-miR-19a-3p | hsa-miR-217 | hsa-miR-27b-3p |
| hsa-miR-150-3p | hsa-miR-18a-5p | hsa-miR-19b-3p | hsa-miR-218-1-3p | hsa-miR-27b-5p |
| hsa-miR-150-5p | hsa-miR-18b-3p | hsa-miR-200a-3p | hsa-miR-218-2-3p | hsa-miR-28-3p |
| hsa-miR-151a-3p | hsa-miR-18b-5p | hsa-miR-200a-5p | hsa-miR-218-5p | hsa-miR-28-5p |
| hsa-miR-151b/151 [symbol] 5p | hsa-miR-1908-3p | hsa-miR-200b-3p | hsa-miR-219a-1-3p | hsa-miR-2909 |
| hsa-miR-152-3p | hsa-miR-1908-5p | hsa-miR-200b-5p | hsa-miR-219a-2-3p | hsa-miR-296-3p |
| hsa-miR-152-5p | hsa-miR-1909-3p | hsa-miR-200c-3p | hsa-miR-219b-3p | hsa-miR-296-5p |
| hsa-miR-1539 | hsa-miR-190a-5p | hsa-miR-200c-5p | hsa-miR-219b-5p | hsa-miR-298 |
| hsa-miR-154-5p | hsa-miR-190b | hsa-miR-202-3p | hsa-miR-22-3p | hsa-miR-299-3p |
| hsa-miR-155-3p | hsa-miR-191-3p | hsa-miR-203a-3p | hsa-miR-22-5p | hsa-miR-299-5p |
| hsa-miR-155-5p | hsa-miR-191-5p | hsa-miR-203a-5p | hsa-miR-221-3p | hsa-miR-29a-3p |
| hsa-miR-1587 | hsa-miR-1910-5p | hsa-miR-203b-5p | hsa-miR-221-5p | hsa-miR-29a-5p |
| hsa-miR-15a-5p | hsa-miR-1911-3p | hsa-miR-204-3p | hsa-miR-222-3p | hsa-miR-29b-1-5p |
| hsa-miR-15b-3p | hsa-miR-1911-5p | hsa-miR-204-5p | hsa-miR-223-3p | hsa-miR-29b-2-5p |
| hsa-miR-15b-5p | hsa-miR-1912 | hsa-miR-205-5p | hsa-miR-223-5p | hsa-miR-29b-3p |
| hsa-miR-16-2-3p | hsa-miR-1913 | hsa-miR-2052 | hsa-miR-224-5p | hsa-miR-29c-3p |
| hsa-miR-16-5p | hsa-miR-1914-3p | hsa-miR-206 | hsa-miR-2276-3p | hsa-miR-29c-5p |
| hsa-miR-17-3p | hsa-miR-1914-5p | hsa-miR-208a-5p | hsa-miR-2277-3p | hsa-miR-301a-3p |
| hsa-miR-17-5p | hsa-miR-1915-3p | hsa-miR-208b-3p | hsa-miR-2355-3p | hsa-miR-301b-5p |
| hsa-miR-181a-2-3p | hsa-miR-192-3p | hsa-miR-20a-5p | hsa-miR-2355-5p | hsa-miR-302a-5p |
| hsa-miR-181a-3p | hsa-miR-192-5p | hsa-miR-20b-5p | hsa-miR-2392 | hsa-miR-302b-3p |
| hsa-miR-181a-5p | hsa-miR-193a-5p | hsa-miR-21-3p | hsa-miR-23a-3p | hsa-miR-302b-5p |
| hsa-miR-181b-2-3p | hsa-miR-193b-3p | hsa-miR-21-5p | hsa-miR-23a-5p | hsa-miR-302d-3p |
| hsa-miR-181b-3p | hsa-miR-193b-5p | hsa-miR-210-3p | hsa-miR-23b-3p | hsa-miR-3064-5p |
| hsa-miR-181b-5p | hsa-miR-194-3p | hsa-miR-210-5p | hsa-miR-23b-5p | hsa-miR-30a-3p |
| hsa-miR-181c-3p | hsa-miR-194-5p | hsa-miR-211-5p | hsa-miR-24-1-5p | hsa-miR-30a-5p |
| hsa-miR-181c-5p | hsa-miR-195-3p | hsa-miR-2110 | hsa-miR-24-2-5p | hsa-miR-30b-3p |
| hsa-miR-181d-3p | hsa-miR-195-5p | hsa-miR-2113 | hsa-miR-24-3p | hsa-miR-30b-5p |
| hsa-miR-181d-5p | hsa-miR-196a-3p | hsa-miR-2114-3p | hsa-miR-2467-5p | hsa-miR-30c-1-3p |
| hsa-miR-182-5p | hsa-miR-196a-5p | hsa-miR-2114-5p | hsa-miR-25-3p | hsa-miR-30c-2-3p |
| hsa-miR-1825 | hsa-miR-196b-3p | hsa-miR-2115-3p | hsa-miR-25-5p | hsa-miR-30c-5p |
| hsa-miR-183-3p | hsa-miR-196b-5p | hsa-miR-2115-5p | hsa-miR-2681-3p | hsa-miR-30d-3p |
| hsa-miR-183-5p | hsa-miR-197-3p | hsa-miR-2116-3p | hsa-miR-2682-3p | hsa-miR-30d-5p |
| hsa-miR-184 | hsa-miR-197-5p | hsa-miR-2116-5p | hsa-miR-2682-5p | hsa-miR-30e-3p |
| hsa-miR-185-3p | hsa-miR-1972 | hsa-miR-2117 | hsa-miR-26a-1-3p | hsa-miR-30e-5p |
| hsa-miR-185-5p | hsa-miR-1976 | hsa-miR-214-3p | hsa-miR-26a-2-3p | hsa-miR-31-3p |
| hsa-miR-186-3p | hsa-miR-198 | hsa-miR-214-5p | hsa-miR-26a-5p | hsa-miR-31-5p |
| hsa-miR-3115 | hsa-miR-3158-3p | hsa-miR-320c | hsa-miR-3605-5p | hsa-miR-3663-3p |
| hsa-miR-3116 | hsa-miR-3161 | hsa-miR-320d | hsa-miR-3609 | hsa-miR-3663-5p |
| hsa-miR-3117-5p | hsa-miR-3162-3p | hsa-miR-320e | hsa-miR-361-3p | hsa-miR-3666 |
| hsa-miR-3120-3p | hsa-miR-3162-5p | hsa-miR-323a-3p | hsa-miR-361-5p | hsa-miR-3667-3p |
| hsa-miR-3121-5p | hsa-miR-3163 | hsa-miR-323a-5p | hsa-miR-3610 | hsa-miR-3667-5p |
| hsa-miR-3122 | hsa-miR-3165 | hsa-miR-323b-3p | hsa-miR-3612 | hsa-miR-3668 |
| hsa-miR-3124-3p | hsa-miR-3168 | hsa-miR-323b-5p | hsa-miR-3613-3p | hsa-miR-3670 |
| hsa-miR-3124-5p | hsa-miR-3170 | hsa-miR-324-3p | hsa-miR-3613-5p | hsa-miR-3674 |
| hsa-miR-3125 | hsa-miR-3171 | hsa-miR-324-5p | hsa-miR-3614-3p | hsa-miR-3675-3p |
| hsa-miR-3126-3p | hsa-miR-3174 | hsa-miR-325 | hsa-miR-3615 | hsa-miR-3677-3p |
| hsa-miR-3126-5p | hsa-miR-3175 | hsa-miR-326 | hsa-miR-3616-3p | hsa-miR-3677-5p |
| hsa-miR-3127-3p | hsa-mIR-3176 | hsa-miR-328-3p | hsa-miR-3617-3p | hsa-miR-3679-3p |
| hsa-miR-3127-5p | hsa-miR-3178 | hsa-miR-328-5p | hsa-miR-3617-5p | hsa-miR-3679-5p |
| hsa-miR-3129-5p | hsa-miR-3179 | hsa-miR-329-3p | hsa-miR-3618 | hsa-miR-3680-3p |
| hsa-miR-3130-3p | hsa-miR-3180-3p | hsa-miR-329-5p | hsa-miR-3619-3p | hsa-miR-3681-3p |
| hsa-miR-3130-5p | hsa-miR-3180-5p | hsa-miR-330-5p | hsa-miR-3619-5p | hsa-mIR-3682-3p |
| hsa-miR-3131 | hsa-miR-3181 | hsa-miR-331-3p | hsa-miR-362-5p | hsa-miR-3683 |
| hsa-miR-3132 | hsa-miR-3182 | hsa-miR-331-5p | hsa-miR-3620-3p | hsa-miR-3684 |
| hsa-miR-3133 | hsa-miR-3183 | hsa-miR-335-3p | hsa-miR-3620-5p | hsa-miR-3685 |
| hsa-miR-3135b | hsa-miR-3185 | hsa-miR-335-5p | hsa-miR-3621 | hsa-miR-3689b-3p |
| hsa-miR-3136-3p | hsa-mIR-3186-3p | hsa-miR-337-3p | hsa-miR-3622b-3p | hsa-miR-3689d |
| hsa-miR-3136-5p | hsa-miR-3187-3p | hsa-miR-337-5p | hsa-miR-363-3p | hsa-miR-369-3p |
| hsa-miR-3138 | hsa-miR-3187-5p | hsa-miR-338-3p | hsa-miR-363-5p | hsa-miR-369-5p |
| hsa-miR-3139 | hsa-miR-3188 | hsa-miR-338-5p | hsa-miR-3646 | hsa-miR-3691-3p |
| hsa-miR-3140-3p | hsa-miR-3189-3p | hsa-miR-339-3p | hsa-miR-3648 | hsa-miR-3691-5p |
| hsa-miR-3141 | hsa-miR-3190-5p | hsa-miR-339-5p | hsa-miR-3649 | hsa-miR-3692-3p |
| hsa-miR-3142 | hsa-miR-3191-5p | hsa-miR-33a-3p | hsa-miR-3651 | hsa-miR-3692-5p |
| hsa-miR-3143 | hsa-miR-3192-3p | hsa-miR-33a-5p | hsa-miR-3652 | hsa-miR-370-3p |
| hsa-miR-3144-3p | hsa-miR-3192-5p | hsa-miR-33b-3p | hsa-miR-3653-3p | hsa-miR-370-5p |
| hsa-miR-3144-5p | hsa-miR-3193 | hsa-miR-33b-5p | hsa-miR-3653-5p | hsa-miR-3713 |
| hsa-miR-3145-3p | hsa-miR-3194-3p | hsa-miR-340-5p | hsa-miR-3654 | hsa-miR-371b-5p |
| hsa-miR-3145-5p | hsa-miR-3195 | hsa-miR-342-3p | hsa-miR-3655 | hsa-miR-373-3p |
| hsa-miR-3146 | hsa-miR-3196 | hsa-miR-342-5p | hsa-miR-3656 | hsa-miR-373-5p |
| hsa-miR-3147 | hsa-miR-3197 | hsa-miR-345-3p | hsa-miR-3658 | hsa-miR-374a-3p |
| hsa-miR-3150b-3p | hsa-miR-3198 | hsa-miR-345-5p | hsa-miR-3659 | hsa-miR-374a-5p |
| hsa-miR-3151-3p | hsa-miR-32-5p | hsa-miR-346 | hsa-miR-365a-3p | hsa-miR-374b-5p |
| hsa-miR-3151-5p | hsa-miR-3200-3p | hsa-miR-34a-5p | hsa-miR-365a-5p | hsa-miR-375 |
| hsa-miR-3152-3p | hsa-miR-3200-5p | hsa-miR-34b-3p | hsa-miR-365b-3p | hsa-miR-376a-2-5p |
| hsa-miR-3153 | hsa-miR-3202 | hsa-miR-34c-3p | hsa-miR-365b-5p | hsa-miR-376a-3p |
| hsa-miR-3154 | hsa-miR-320a | hsa-miR-34c-5p | hsa-miR-3661 | hsa-miR-376a-5p |
| hsa-miR-3157-3p | hsa-miR-320b | hsa-miR-3605-3p | hsa-miR-3662 | hsa-miR-376b-3p |
| hsa-miR-376b-5p | hsa-miR-3939 | hsa-miR-4268 | hsa-miR-4322 | hsa-miR-4464 |
| hsa-miR-376c-3p | hsa-miR-3940-3p | hsa-miR-4271 | hsa-miR-4324 | hsa-miR-4465 |
| hsa-miR-376c-5p | hsa-miR-3941 | hsa-miR-4273 | hsa-miR-4325 | hsa-miR-4466 |
| hsa-miR-377-3p | hsa-miR-3942-3p | hsa-miR-4274 | hsa-miR-4326 | hsa-miR-4469 |
| hsa-miR-377-5p | hsa-miR-3942-5p | hsa-miR-4275 | hsa-miR-4327 | hsa-miR-4472 |
| hsa-miR-378a-3p | hsa-miR-3943 | hsa-miR-4279 | hsa-miR-4329 | hsa-miR-4474-5p |
| hsa-miR-378a-5p | hsa-miR-3944-3p | hsa-miR-4280 | hsa-miR-433-3p | hsa-miR-4476 |
| hsa-miR-378c | hsa-miR-3944-5p | hsa-miR-4282 | hsa-miR-4417 | hsa-miR-4477b |
| hsa-miR-378d | hsa-miR-3945 | hsa-miR-4284 | hsa-miR-4418 | hsa-miR-4478 |
| hsa-miR-378f | hsa-miR-3960 | hsa-miR-4285 | hsa-miR-4419b | hsa-miR-4479 |
| hsa-miR-378g | hsa-miR-3972 | hsa-miR-4286 | hsa-miR-4422 | hsa-miR-4480 |
| hsa-miR-378h | hsa-miR-3974 | hsa-miR-4287 | hsa-miR-4423-3p | hsa-miR-4481 |
| hsa-miR-3781 | hsa-miR-3976 | hsa-miR-4288 | hsa-miR-4425 | hsa-miR-4482-5p |
| hsa-miR-378j | hsa-miR-3978 | hsa-miR-429 | hsa-miR-4426 | hsa-miR-4484 |
| hsa-miR-379-3p | hsa-miR-409-3p | hsa-miR-4290 | hsa-miR-4428 | hsa-miR-4485-3p |
| hsa-miR-379-5p | hsa-miR-409-5p | hsa-miR-4291 | hsa-miR-4429 | hsa-miR-4488 |
| hsa-miR-380-5p | hsa-miR-410-3p | hsa-miR-4292 | hsa-miR-4430 | hsa-miR-4489 |
| hsa-miR-381-3p | hsa-miR-410-5p | hsa-miR-4293 | hsa-miR-4433a-5p | hsa-miR-4491 |
| hsa-miR-382-3p | hsa-miR-411-3p | hsa-miR-4295 | hsa-miR-4433b-3p | hsa-miR-4492 |
| hsa-miR-382-5p | hsa-miR-411-5p | hsa-miR-4296 | hsa-miR-4433b-5p | hsa-miR-4493 |
| hsa-miR-383-3p | hsa-miR-412-3p | hsa-miR-4297 | hsa-miR-4435 | hsa-miR-4494 |
| hsa-miR-383-5p | hsa-miR-412-5p | hsa-miR-4300 | hsa-miR-4436b-3p | hsa-miR-4495 |
| hsa-miR-384 | hsa-miR-421 | hsa-miR-4301 | hsa-miR-4436b-5p | hsa-miR-4496 |
| hsa-miR-3907 | hsa-miR-423-3p | hsa-miR-4302 | hsa-miR-4440 | hsa-miR-4498 |
| hsa-miR-3910 | hsa-miR-423-5p | hsa-miR-4303 | hsa-miR-4442 | hsa-miR-4499 |
| hsa-miR-3913-3p | hsa-miR-424-3p | hsa-miR-4304 | hsa-miR-4443 | hsa-miR-449b-3p |
| hsa-miR-3913-5p | hsa-miR-424-5p | hsa-miR-4305 | hsa-miR-4444 | hsa-miR-449b-5p |
| hsa-miR-3914 | hsa-miR-425-3p | hsa-miR-4309 | hsa-miR-4446-5p | hsa-miR-449c-3p |
| hsa-miR-3915 | hsa-miR-425-5p | hsa-miR-431-3p | hsa-miR-4448 | hsa-miR-449c-5p |
| hsa-miR-3916 | hsa-miR-4251 | hsa-miR-431-5p | hsa-miR-4449 | hsa-miR-4501 |
| hsa-miR-3917 | hsa-miR-4252 | hsa-miR-4310 | hsa-miR-4450 | hsa-miR-4502 |
| hsa-miR-3919 | hsa-miR-4254 | hsa-miR-4311 | hsa-miR-4451 | hsa-miR-4505 |
| hsa-miR-3921 | hsa-miR-4255 | hsa-miR-4312 | hsa-miR-4452 | hsa-miR-4507 |
| hsa-miR-3922-5p | hsa-miR-4257 | hsa-miR-4313 | hsa-miR-4454 | hsa-miR-4508 |
| hsa-miR-3923 | hsa-miR-4258 | hsa-miR-4316 | hsa-miR-4456 | hsa-miR-450a-1-3p |
| hsa-miR-3925-5p | hsa-miR-4259 | hsa-miR-4317 | hsa-miR-4457 | hsa-miR-450a-2-3p |
| hsa-miR-3927-3p | hsa-miR-4260 | hsa-miR-4319 | hsa-miR-4458 | hsa-miR-450a-5p |
| hsa-miR-3928-3p | hsa-miR-4261 | hsa-miR-432-3p | hsa-miR-4459 | hsa-miR-450b-3p |
| hsa-miR-3928-5p | hsa-miR-4262 | hsa-miR-432-5p | hsa-miR-4461 | hsa-miR-450b-5p |
| hsa-miR-3934-5p | hsa-miR-4266 | hsa-miR-4320 | hsa-miR-4462 | hsa-miR-4510 |
| hsa-miR-3938 | hsa-miR-4267 | hsa-miR-4321 | hsa-miR-4463 | hsa-miR-4512 |
| hsa-miR-4514 | hsa-miR-4645-3p | hsa-miR-4687-3p | hsa-miR-4727-5p | hsa-miR-4769-3p |
| hsa-miR-4515 | hsa-miR-4646-3p | hsa-miR-4687-5p | hsa-miR-4728-3p | hsa-miR-4770 |
| hsa-miR-4516 | hsa-miR-4646-5p | hsa-miR-4688 | hsa-miR-4729 | hsa-miR-4771 |
| hsa-miR-4518 | hsa-miR-4647 | hsa-miR-4690-5p | hsa-miR-4731-3p | hsa-miR-4772-3p |
| hsa-miR-451a | hsa-miR-4649-5p | hsa-miR-4691-3p | hsa-miR-4732-3p | hsa-miR-4774-3p |
| hsa-miR-451b | hsa-miR-4650-5p | hsa-miR-4691-5p | hsa-miR-4732-5p | hsa-miR-4775 |
| hsa-miR-452-3p | hsa-miR-4651 | hsa-miR-4693-5p | hsa-miR-4733-3p | hsa-miR-4776-5p |
| hsa-miR-452-5p | hsa-miR-4652-3p | hsa-miR-4694-5p | hsa-miR-4734 | hsa-miR-4778-3p |
| hsa-miR-4520-3p | hsa-miR-4652-5p | hsa-miR-4695-3p | hsa-miR-4735-5p | hsa-miR-4778-5p |
| hsa-miR-4522 | hsa-miR-4653-3p | hsa-miR-4695-5p | hsa-miR-4736 | hsa-miR-4780 |
| hsa-miR-4523 | hsa-miR-4654 | hsa-miR-4698 | hsa-miR-4737 | hsa-miR-4781-3p |
| hsa-miR-4524a-3p | hsa-miR-4655-3p | hsa-miR-4699-3p | hsa-miR-4738-5p | hsa-miR-4781-5p |
| hsa-miR-4524b-3p | hsa-miR-4655-5p | hsa-miR-4700-3p | hsa-miR-4739 | hsa-miR-4782-5p |
| hsa-miR-4529-3p | hsa-miR-4658 | hsa-miR-4700-5p | hsa-miR-4740-3p | hsa-miR-4783-3p |
| hsa-miR-4529-5p | hsa-miR-4659a-3p | hsa-miR-4701-5p | hsa-miR-4740-5p | hsa-miR-4783-5p |
| hsa-miR-4530 | hsa-miR-466 | hsa-miR-4703-3p | hsa-miR-4741 | hsa-miR-4784 |
| hsa-miR-4531 | hsa-miR-4660 | hsa-miR-4703-5p | hsa-miR-4743-3p | hsa-miR-4785 |
| hsa-miR-4532 | hsa-miR-4661-5p | hsa-miR-4704-5p | hsa-miR-4743-5p | hsa-miR-4787-3p |
| hsa-miR-4533 | hsa-miR-4664-3p | hsa-miR-4706 | hsa-miR-4745-3p | hsa-miR-4788 |
| hsa-miR-4534 | hsa-miR-4664-5p | hsa-miR-4707-3p | hsa-miR-4745-5p | hsa-miR-4789-3p |
| hsa-miR-4535 | hsa-miR-4665-3p | hsa-miR-4707-5p | hsa-miR-4746-3p | hsa-miR-4791 |
| hsa-miR-4537 | hsa-miR-4665-5p | hsa-miR-4709-5p | hsa-miR-4746-5p | hsa-miR-4792 |
| hsa-miR-4538 | hsa-miR-4667-3p | hsa-miR-4711-3p | hsa-miR-4747-3p | hsa-miR-4793-3p |
| hsa-miR-4539 | hsa-miR-4667-5p | hsa-miR-4711-5p | hsa-miR-4748 | hsa-miR-4793-5p |
| hsa-miR-454-3p | hsa-miR-4668-5p | hsa-miR-4712-5p | hsa-miR-4749-5p | hsa-miR-4795-3p |
| hsa-miR-454-5p | hsa-miR-4670-3p | hsa-miR-4713-3p | hsa-miR-4750-5p | hsa-miR-4796-3p |
| hsa-miR-4540 | hsa-miR-4671-5p | hsa-miR-4713-5p | hsa-miR-4751 | hsa-miR-4796-5p |
| hsa-miR-455-3p | hsa-miR-4673 | hsa-miR-4714-3p | hsa-miR-4752 | hsa-miR-4798-3p |
| hsa-miR-455-5p | hsa-miR-4676-3p | hsa-miR-4714-5p | hsa-miR-4753-3p | hsa-miR-4798-5p |
| hsa-miR-4632-5p | hsa-miR-4676-5p | hsa-miR-4716-3p | hsa-miR-4753-5p | hsa-miR-4799-3p |
| hsa-miR-4634 | hsa-miR-4677-3p | hsa-miR-4716-5p | hsa-miR-4754 | hsa-miR-4800-3p |
| hsa-miR-4635 | hsa-miR-4677-5p | hsa-miR-4718 | hsa-miR-4755-5p | hsa-miR-4800-5p |
| hsa-miR-4636 | hsa-miR-4679 | hsa-miR-4719 | hsa-miR-4758-5p | hsa-miR-4802-3p |
| hsa-miR-4638-3p | hsa-miR-4680-5p | hsa-miR-4721 | hsa-miR-4759 | hsa-miR-4803 |
| hsa-miR-4638-5p | hsa-miR-4681 | hsa-miR-4722-3p | hsa-miR-4760-5p | hsa-miR-4804-3p |
| hsa-miR-4639-3p | hsa-miR-4682 | hsa-miR-4723-5p | hsa-miR-4763-5p | hsa-miR-4804-5p |
| hsa-miR-4639-5p | hsa-miR-4683 | hsa-miR-4724-5p | hsa-miR-4764-3p | hsa-miR-483-3p |
| hsa-miR-4640-5p | hsa-miR-4684-3p | hsa-miR-4725-3p | hsa-miR-4764-5p | hsa-miR-483-5p |
| hsa-miR-4641 | hsa-miR-4685-3p | hsa-miR-4725-5p | hsa-miR-4765 | hsa-miR-484 |
| hsa-miR-4642 | hsa-miR-4685-5p | hsa-miR-4726-3p | hsa-miR-4767 | hsa-miR-485-3p |
| hsa-miR-4644 | hsa-miR-4686 | hsa-miR-4726-5p | hsa-miR-4768-5p | hsa-miR-485-5p |
| hsa-miR-486-3p | hsa-miR-504-3p | hsa-miR-518e-3p | hsa-miR-548ba | hsa-miR-5691 |
| hsa-miR-486-5p | hsa-miR-504-5p | hsa-miR-518f-3p | hsa-miR-548d-3p | hsa-miR-5692a |
| hsa-miR-487a-3p | hsa-miR-5047 | hsa-miR-5190 | hsa-miR-548e-3p | hsa-miR-5694 |
| hsa-miR-487a-5p | hsa-miR-505-3p | hsa-miR-5191 | hsa-miR-548f-3p | hsa-miR-5695 |
| hsa-miR-487b-3p | hsa-miR-505-5p | hsa-miR-5192 | hsa-miR-5481 | hsa-miR-5698 |
| hsa-miR-487b-5p | hsa-miR-506-5p | hsa-miR-5193 | hsa-miR-548t-5p | hsa-miR-5699-3p |
| hsa-miR-488-3p | hsa-miR-507 | hsa-miR-5194 | hsa-miR-548v | hsa-miR-5699-5p |
| hsa-miR-488-5p | hsa-miR-508-3p | hsa-miR-5195-3p | hsa-miR-548w | hsa-miR-5700 |
| hsa-miR-490-3p | hsa-miR-508-5p | hsa-miR-5195-5p | hsa-miR-549a | hsa-miR-5701 |
| hsa-miR-490-5p | hsa-miR-5087 | hsa-miR-5196-3p | hsa-miR-551a | hsa-miR-5702 |
| hsa-miR-491-3p | hsa-miR-5088-3p | hsa-miR-5196-5p | hsa-miR-551b-5p | hsa-miR-5703 |
| hsa-miR-491-5p | hsa-miR-5088-5p | hsa-miR-5197-3p | hsa-miR-552-5p | hsa-miR-5704 |
| hsa-miR-493-3p | hsa-miR-5089-3p | hsa-miR-5197-5p | hsa-miR-553 | hsa-miR-5705 |
| hsa-miR-493-5p | hsa-miR-5089-5p | hsa-miR-519e-5p | hsa-miR-554 | hsa-miR-5706 |
| hsa-miR-494-3p | hsa-miR-509-3p | hsa-miR-520a-3p | hsa-miR-555 | hsa-miR-5707 |
| hsa-miR-495-3p | hsa-miR-509-5p | hsa-miR-520d-3p | hsa-miR-556-3p | hsa-miR-572 |
| hsa-miR-495-5p | hsa-miR-5090 | hsa-miR-520g-5p | hsa-miR-5571-5p | hsa-miR-573 |
| hsa-miR-496 | hsa-miR-5091 | hsa-miR-520h | hsa-miR-5572 | hsa-miR-574-3p |
| hsa-miR-497-3p | hsa-miR-5092 | hsa-miR-521 | hsa-miR-558 | hsa-miR-574-5p |
| hsa-miR-497-5p | hsa-miR-5095 | hsa-miR-524-3p | hsa-miR-5580-3p | hsa-miR-575 |
| hsa-miR-4999-3p | hsa-miR-510-3p | hsa-miR-525-3p | hsa-miR-5583-5p | hsa-miR-576-3p |
| hsa-miR-5001-3p | hsa-miR-510-5p | hsa-miR-532-3p | hsa-miR-5584-3p | hsa-miR-576-5p |
| hsa-miR-5001-5p | hsa-miR-5100 | hsa-miR-532-5p | hsa-miR-5584-5p | hsa-miR-578 |
| hsa-miR-5002-3p | hsa-miR-511-3p | hsa-miR-539-3p | hsa-miR-5585-5p | hsa-miR-5787 |
| hsa-miR-5003-3p | hsa-miR-511-5p | hsa-miR-539-5p | hsa-miR-5586-3p | hsa-miR-579-3p |
| hsa-miR-5003-5p | hsa-miR-512-5p | hsa-miR-541-5p | hsa-miR-5586-5p | hsa-miR-580-3p |
| hsa-miR-5004-3p | hsa-miR-513a-5p | hsa-miR-542-3p | hsa-miR-5587-3p | hsa-miR-580-5p |
| hsa-miR-5006-3p | hsa-miR-514a-3p | hsa-miR-542-5p | hsa-miR-5587-5p | hsa-miR-582-3p |
| hsa-miR-5006-5p | hsa-miR-514a-5p | hsa-miR-543 | hsa-miR-5588-5p | hsa-miR-582-5p |
| hsa-miR-5007-3p | hsa-miR-514b-3p | hsa-miR-544a | hsa-miR-5589-5p | hsa-miR-584-3p |
| hsa-miR-5009-3p | hsa-miR-515-3p | hsa-miR-548ac | hsa-miR-561-3p | hsa-miR-584-5p |
| hsa-miR-5009-5p | hsa-miR-516a-3p | hsa-miR-548ad-3p | hsa-miR-561-5p | hsa-miR-585-3p |
| hsa-miR-500a-3p | hsa-miR-516a-5p | hsa-miR-548ao-3p | hsa-miR-564 | hsa-miR-585-5p |
| hsa-miR-501-3p | hsa-miR-5186 | hsa-miR-548ao-5p | hsa-miR-566 | hsa-miR-586 |
| hsa-miR-501-5p | hsa-miR-5187-3p | hsa-miR-548ap-3p | hsa-miR-567 | hsa-miR-588 |
| hsa-miR-5010-3p | hsa-miR-5187-5p | 548t-3p/548aa | hsa-miR-5680 | hsa-miR-589-3p |
| hsa-miR-5010-5p | hsa-miR-5188 | hsa-miR-548at-5p | hsa-miR-5682 | hsa-miR-589-5p |
| hsa-miR-5011-5p | hsa-miR-5189-3p | hsa-miR-548av-5p/548k | hsa-miR-5683 | hsa-miR-590-3p |
| hsa-miR-502-3p | hsa-miR-518a-3p | hsa-miR-548aw | hsa-miR-5684 | hsa-miR-591 |
| hsa-miR-503-3p | hsa-miR-518b | hsa-miR-548ay-3p | hsa-miR-5685 | hsa-miR-592 |
| hsa-miR-503-5p | hsa-miR-518d-3p | hsa-miR-548az-5p | hsa-miR-5690 | hsa-miR-593-5p |
| hsa-miR-596 | hsa-miR-616-3p | hsa-miR-6506-5p | hsa-miR-6719-3p | hsa-miR-6748-5p |
| hsa-miR-597-3p | hsa-miR-616-5p | hsa-miR-6507-3p | hsa-miR-6720-3p | hsa-miR-6749-5p |
| hsa-miR-597-5p | hsa-miR-6165 | hsa-miR-6507-5p | hsa-miR-6720-5p | hsa-miR-675-3p |
| hsa-miR-598-3p | hsa-miR-617 | hsa-miR-6508-5p | hsa-miR-6721-5p | hsa-miR-675-5p |
| hsa-miR-598-5p | hsa-miR-618 | hsa-miR-651-5p | hsa-miR-6722-5p | hsa-miR-6750-3p |
| hsa-miR-601 | hsa-miR-619-3p | hsa-miR-6510-5p | hsa-miR-6724-5p | hsa-miR-6750-5p |
| hsa-miR-602 | hsa-miR-622 | hsa-miR-6512-3p | hsa-miR-6726-3p | hsa-miR-6751-3p |
| hsa-miR-605-5p | hsa-miR-623 | hsa-miR-6512-5p | hsa-miR-6726-5p | hsa-miR-6751-5p |
| hsa-miR-606 | hsa-miR-624-3p | hsa-miR-6513-3p | hsa-miR-6727-3p | hsa-miR-6752-3p |
| hsa-miR-6068 | hsa-miR-625-3p | hsa-miR-6513-5p | hsa-miR-6727-5p | hsa-miR-6752-5p |
| hsa-miR-6069 | hsa-miR-625-5p | hsa-miR-6514-3p | hsa-miR-6728-5p | hsa-miR-6754-3p |
| hsa-miR-607 | hsa-miR-626 | hsa-miR-6515-3p | hsa-miR-6729-3p | hsa-miR-6754-5p |
| hsa-miR-6070 | hsa-miR-628-3p | hsa-miR-6515-5p | hsa-miR-6729-5p | hsa-miR-6755-5p |
| hsa-miR-6074 | hsa-miR-628-5p | hsa-miR-652-3p | hsa-miR-6730-3p | hsa-miR-6756-3p |
| hsa-miR-6075 | hsa-miR-629-3p | hsa-miR-653-3p | hsa-miR-6730-5p | hsa-miR-6756-5p |
| hsa-miR-6078 | hsa-miR-629-5p | hsa-miR-654-3p | hsa-miR-6731-3p | hsa-miR-6757-5p |
| hsa-miR-6079 | hsa-miR-630 | hsa-miR-654-5p | hsa-miR-6731-5p | hsa-miR-6758-3p |
| hsa-miR-608 | hsa-miR-631 | hsa-miR-655-3p | hsa-miR-6732-3p | hsa-miR-6758-5p |
| hsa-miR-6080 | hsa-miR-634 | hsa-miR-656-3p | hsa-miR-6733-5p | hsa-miR-676-3p |
| hsa-miR-6081 | hsa-miR-635 | hsa-miR-657 | hsa-miR-6734-3p | hsa-miR-676-5p |
| hsa-miR-6082 | hsa-miR-636 | hsa-miR-658 | hsa-miR-6734-5p | hsa-miR-6760-3p |
| hsa-miR-6084 | hsa-miR-637 | hsa-miR-659-3p | hsa-miR-6735-3p | hsa-miR-6760-5p |
| hsa-miR-6087 | hsa-miR-638 | hsa-miR-659-5p | hsa-miR-6735-5p | hsa-miR-6761-3p |
| hsa-miR-6088 | hsa-miR-639 | hsa-miR-660-3p | hsa-miR-6736-3p | hsa-miR-6761-5p |
| hsa-miR-6090 | hsa-miR-641 | hsa-miR-660-5p | hsa-miR-6737-3p | hsa-miR-6762-5p |
| hsa-miR-610 | hsa-miR-642a-5p | hsa-miR-662 | hsa-miR-6737-5p | hsa-miR-6763-3p |
| hsa-miR-611 | hsa-miR-643 | hsa-miR-6638 | hsa-miR-6738-5p | hsa-miR-6763-5p |
| hsa-miR-612 | hsa-miR-644a | hsa-miR-663b | hsa-miR-6739-5p | hsa-miR-6764-3p |
| hsa-miR-6124 | hsa-miR-647 | hsa-miR-664a-3p | hsa-miR-6740-3p | hsa-miR-6764-5p |
| hsa-miR-6125 | hsa-miR-649 | hsa-miR-664a-5p | hsa-miR-6740-5p | hsa-miR-6765-3p |
| hsa-miR-6126 | hsa-miR-6499-5p | hsa-miR-664b-5p | hsa-miR-6741-3p | hsa-miR-6766-3p |
| hsa-miR-6128 | hse-miR-6500-3p | hsa-miR-665 | hsa-miR-6741-5p | hsa-miR-6766-5p |
| hsa-miR-6129 | hsa-miR-6500-5p | hsa-miR-668-3p | hsa-miR-6742-5p | hsa-miR-6767-3p |
| hsa-miR-613 | hsa-miR-6501-5p | hsa-miR-670-3p | hsa-miR-6743-3p | hsa-miR-6767-5p |
| hsa-miR-6130 | hsa-miR-6502-5p | hsa-miR-671-3p | hsa-miR-6743-5p | hsa-miR-6768-3p |
| hsa-miR-6131 | hsa-miR-6503-3p | hsa-miR-671-5p | hsa-miR-6744-3p | hsa-miR-6768-5p |
| hsa-miR-6132 | hsa-miR-6503-5p | hsa-miR-6715b-3p | hsa-miR-6745 | hsa-miR-6769a-5p |
| hsa-miR-6133 | hsa-miR-6504-3p | hsa-miR-6715b-5p | hsa-miR-6746-3p | hsa-miR-6770-3p |
| hsa-miR-614 | hsa-miR-6504-5p | hsa-miR-6716-3p | hsa-miR-6746-5p | hsa-miR-6771-3p |
| hsa-miR-615-3p | hsa-miR-6505-3p | hsa-miR-6716-5p | hsa-miR-6747-3p | hsa-miR-6771-5p |
| hsa-miR-615-5p | hsa-miR-6505-5p | hsa-miR-6717-5p | hsa-miR-6747-5p | hsa-miR-6772-5p |
| hsa-miR-6775-3p | hsa-miR-6802-3p | hsa-miR-6831-3p | hsa-miR-6864-3p | hsa-miR-6893-3p |
| hsa-miR-6775-5p | hsa-miR-6802-5p | hsa-miR-6831-5p | hsa-miR-6864-5p | hsa-miR-6893-5p |
| hsa-miR-6776-3p | hsa-miR-6803-3p | hsa-miR-6832-3p | hsa-miR-6865-3p | hsa-miR-6894-3p |
| hsa-miR-6777-5p | hsa-miR-6803-5p | hsa-miR-6832-5p | hsa-miR-6865-5p | hsa-miR-6894-5p |
| hsa-miR-6778-5p | hsa-miR-6805-3p | hsa-miR-6833-3p | hsa-miR-6866-3p | hsa-miR-6895-3p |
| hsa-miR-6779-3p | hsa-miR-6805-5p | hsa-miR-6833-5p | hsa-miR-6866-5p | hsa-miR-7-1-3p |
| hsa-miR-6779-5p | hsa-miR-6806-3p | hsa-miR-6834-5p | hsa-miR-6867-5p | hsa-miR-7-2-3p |
| hsa-miR-6780a-5p | hsa-miR-6807-5p | hsa-miR-6835-5p | hsa-miR-6869-3p | hsa-miR-7-5p |
| hsa-miR-6780b-3p | hsa-miR-6808-5p | hsa-miR-6836-3p | hsa-miR-6869-5p | hsa-miR-708-3p |
| hsa-miR-6780b-5p | hsa-miR-6809-3p | hsa-miR-6839-3p | hsa-miR-6870-3p | hsa-miR-708-5p |
| hsa-mIR-6781-5p | hsa-miR-6809-5p | hsa-miR-6839-5p | hsa-miR-6870-5p | hsa-miR-7106-3p |
| hsa-miR-6782-5p | hsa-miR-6810-3p | hsa-miR-6840-5p | hsa-miR-6871-3p | hsa-miR-7106-5p |
| hsa-miR-6783-3p | hsa-miR-6811-5p | hsa-miR-6842-3p | hsa-miR-6871-5p | hsa-miR-7107-3p |
| hsa-miR-6783-5p | hsa-miR-6812-3p | hsa-miR-6842-5p | hsa-miR-6872-3p | hsa-miR-7107-5p |
| hsa-miR-6784-3p | hsa-miR-6813-3p | hsa-miR-6843-3p | hsa-miR-6873-3p | hsa-miR-7108-3p |
| hsa-miR-6785-3p | hsa-miR-6813-5p | hsa-miR-6846-3p | hsa-miR-6874-5p | hsa-miR-7108-5p |
| hsa-miR-6785-5p | hsa-miR-6814-3p | hsa-miR-6846-5p | hsa-miR-6875-3p | hsa-miR-7109-3p |
| hsa-miR-6786-3p | hsa-miR-6814-5p | hsa-miR-6847-3p | hsa-miR-6877-3p | hsa-miR-711 |
| hsa-miR-6786-5p | hsa-miR-6815-5p | hsa-miR-6848-5p | hsa-miR-6877-5p | hsa-miR-7110-3p |
| hsa-miR-6787-3p | hsa-miR-6816-3p | hsa-miR-6850-3p | hsa-miR-6878-5p | hsa-miR-7110-5p |
| hsa-miR-6787-5p | hsa-miR-6817-3p | hsa-miR-6850-5p | hsa-miR-6879-3p | hsa-miR-7113-5p |
| hsa-miR-6788-3p | hsa-miR-6817-5p | hsa-miR-6851-3p | hsa-miR-6879-5p | hsa-miR-7114-5p |
| hsa-miR-6788-5p | hsa-miR-6818-3p | hsa-miR-6852-3p | hsa-miR-6881-3p | hsa-miR-7152-3p |
| hsa-miR-6789-5p | hsa-miR-6818-5p | hsa-miR-6853-3p | hsa-miR-6881-5p | hsa-miR-7152-5p |
| hsa-miR-6790-5p | hsa-miR-6819-5p | hsa-miR-6853-5p | hsa-miR-6882-5p | hsa-miR-7153-3p |
| hsa-miR-6791-3p | hsa-miR-6821-3p | hsa-miR-6854-3p | hsa-miR-6883-3p | hsa-miR-7153-5p |
| hsa-miR-6791-5p | hsa-miR-6821-5p | hsa-miR-6854-5p | hsa-miR-6883-5p | hsa-miR-7155-5p |
| hsa-miR-6792-3p | hsa-miR-6822-5p | hsa-miR-6855-3p | hsa-miR-6884-3p | hsa-miR-7156-3p |
| hsa-miR-6792-5p | hsa-miR-6823-3p | hsa-miR-6855-5p | hsa-miR-6884-5p | hsa-miR-7156-5p |
| hsa-miR-6793-3p | hsa-miR-6823-5p | hsa-miR-6856-3p | hsa-miR-6885-3p | hsa-miR-7157-3p |
| hsa-miR-6793-5p | hsa-miR-6824-5p | hsa-miR-6856-5p | hsa-miR-6885-5p | hsa-miR-7158-3p |
| hsa-miR-6794-3p | hsa-miR-6825-3p | hsa-miR-6857-3p | hsa-miR-6886-3p | hsa-miR-7159-3p |
| hsa-miR-6795-5p | hsa-miR-6825-5p | hsa-miR-6857-5p | hsa-miR-6887-3p | hsa-miR-7161-3p |
| hsa-miR-6796-3p | hsa-miR-6826-3p | hsa-miR-6858-5p | hsa-miR-6887-5p | hsa-miR-7162-5p |
| hsa-miR-6796-5p | hsa-miR-6826-5p | hsa-miR-6859-5p | hsa-miR-6888-5p | hsa-miR-718 |
| hsa-miR-6797-5p | hsa-miR-6827-3p | hsa-miR-6860 | hsa-miR-6889-3p | hsa-miR-744-3p |
| hsa-miR-6798-3p | hsa-miR-6828-3p | hsa-miR-6861-3p | hsa-miR-6889-5p | hsa-miR-744-5p |
| hsa-miR-6798-5p | hsa-miR-6828-5p | hsa-miR-6861-5p | hsa-miR-6890-5p | hsa-miR-758-3p |
| hsa-miR-6800-5p | hsa-miR-6829-3p | hsa-miR-6862-3p | hsa-miR-6891-3p | hsa-miR-758-5p |
| hsa-miR-6801-3p | hsa-miR-6830-3p | hsa-miR-6862-5p | hsa-miR-6892-3p | hsa-miR-760 |
| hsa-miR-6801-5p | hsa-miR-6830-5p | hsa-miR-6863 | hsa-miR-6892-5p | hsa-miR-761 |
| hsa-miR-762 | hsa-miR-7851-3p | hsa-miR-8066 | hsa-miR-875-3p | hsa-miR-93-5p |
| hsa-miR-764 | hsa-miR-7853-5p | hsa-miR-8068 | hsa-miR-875-5p | hsa-miR-933 |
| hsa-miR-7641 | hsa-miR-7854-3p | hsa-miR-8069 | hsa-miR-876-5p | hsa-miR-934 |
| hsa-miR-765 | hsa-miR-7855-5p | hsa-miR-8070 | hsa-miR-877-3p | hsa-miR-935 |
| hsa-miR-766-3p | hsa-miR-7856-5p | hsa-miR-8071 | hsa-miR-877-5p | hsa-miR-936 |
| hsB-miR-766-5p | hsa-miR-7973 | hsa-miR-8072 | hsa-miR-885-3p | hsa-miR-937-3p |
| hsa-miR-767-3p | hsa-miR-7974 | hsa-miR-8074 | hsa-miR-885-5p | hsa-miR-940 |
| hsa-miR-767-5p | hsa-miR-7975 | hsa-miR-8075 | hsa-miR-887-3p | hsa-miR-941 |
| hsa-miR-769-3p | hsa-miR-7976 | hsa-miR-8077 | hsa-miR-887-5p | hsa-miR-942-3p |
| hsa-miR-769-5p | hsa-miR-7977 | hsa-miR-8079 | hsa-miR-888-3p | hsa-miR-942-5p |
| hsa-miR-770-5p | hsa-miR-802 | hsa-miR-8081 | hsa-miR-889-3p | hsa-miR-943 |
| hsa-miR-7702 | hsa-miR-8052 | hsa-miR-8082 | hsa-miR-889-5p | hsa-miR-944 |
| hsa-miR-7703 | hsa-miR-8053 | hsa-miR-8083 | hsa-miR-890 | hsa-miR-95-3p |
| hsa-miR-7704 | hsa-miR-8054 | hsa-miR-8085 | hsa-miR-891a-5p | hsa-miR-95-5p |
| hsa-miR-7705 | hsa-miR-8055 | hsa-miR-8086 | hsa-miR-892b | hsa-miR-96-5p |
| hsa-miR-7706 | hsa-miR-8056 | hsa-miR-8087 | hsa-miR-9-5p | hsa-miR-98-3p |
| hsa-miR-7843-3p | hsa-miR-8057 | hsa-miR-8088 | hsa-miR-921 | hsa-miR-98-5p |
| hsa-miR-7845-5p | hsa-miR-8059 | hsa-miR-8089 | hsa-miR-922 | hsa-miR-99a-3p |
| hsa-miR-7846-3p | hsa-miR-8060 | hsa-miR-8485 | hsa-miR-92a-1-5p | hsa-miR-99a-5p |
| hsa-miR-7847-3p | hsa-miR-8062 | hsa-miR-873-3p | hsa-miR-92a-3p | hsa-miR-99b-3p |
| hsa-miR-7848-3p | hsa-miR-8063 | hsa-miR-873-5p | hsa-miR-92b-3p | hsa-miR-99b-5p |
| hsa-miR-7849-3p | hsa-miR-8064 | hsa-miR-874-3p | hsa-miR-92b-5p | |
| hsa-miR-7850-5p | hsa-miR-8065 | hsa-miR-874-5p | hsa-miR-93-3p | |

[symbol]

indicates data missing or illegible when filed

Most preferred miRNAs of EV100 and EV500 samples are shown in Table 6, which comprises miRNA that are present in both EV100 and EV500, and differentially expressed miRNAs.

TABLE 6

| EV100 | EV500 |
|---|---|
| (exosomes) | (microvesicles) |
| miR-199a-3p | miR-199a-3p |
| miR-143-3p | miR-125b-5p |
| miR-21-5p | miR-143-3p |
| miR-125b-5p | miR-21-5p |
| let-7b-5p | let-7i-5p |
| miR-29a-3p | let-7b-5p |
| let-71-5p | miR-125a-5p |
| miR-125a-5p | let-7a-5p |
| miR-16-5p | miR-29a-3p |
| miR-221-3p | let-7f-5p |
| miR-432-5p | miR-16-5p |
| miR-127-3p | miR-127-3p |
| miR-382-5p | miR-221-3p |
| miR-146a-5p | miR-26a-5p |
| miR-431-5p | miR-432-5p |
| let-7a-5p | miR-431-5p |
| miR-199b-3p | miR-126-3p |
| miR-100-5p | miR-181a-5p |
| miR-6504-3p | let-7e-5p |
| miR-26a-5p | miR-382-5p |

For example, the 20 most abundant miRNAs in EV100 and EV500 are listed in Table 6. Eighty percent of the miRNA species are shared between the EV100 and EV500, pointing to the commonality between these vesicular fractions. Interestingly, 20% of the most abundant miRNAs were unique. From this list, there are several miRNAs that show promise for the treatment of inflammatory diseases, including COVID-19.

Our results are in line with previous reports that demonstrated that e.g. micro-RNA 21-5p had a protective role in lung epithelial cells against oxidative stress-induced cell death [12]; and let-7b has been shown to generate the immunosuppressive M2 phenotype in renal macrophages [13]. Without being bound by theory, the miRNAs identified in EV100 and EV500 of Table 5 are thought to provide, at least in part, the anti-inflammatory effect observed.

The presence of these and other miRNAs involved in reducing inflammation suggest that EV100s and EV500s will have a therapeutic effect on inflammatory diseases, such as COVID-19. Experiments using the native EVs, as well as EVs loaded with specific miRNAs or miRNAs depleted from the particles will help to determine which miRNAs are exerting their anti-inflammatory effects and may eventually provide relief for a number of inflammatory diseases, including joint pain and inflammation/rheumatoid arthritis, brain injury and migraine, psoriasis, rosacea, neuropathy and COVID-19. Additionally, the native EVs, as well as EVs loaded with specific miRNAs or miRNAs depleted from the particles may reduce aging-related skin changes such as wrinkles and laxity.

### Example 13: Safety and Toxicity of Umbilical Cord High Muse Cell MSCs and their Exosomes

### Experimental Animals and Husbandry

A total of 50 Balb/c mice (13 weeks old) of both sexes were selected for use in the study based on adequate body weight and absence of clinical signs of disease or injuries. The animals were kept in polypropylene cages in a temperature-controlled room at 21-23° C. and relative humidity of 30%-70%, a 12-h light/dark cycle, and free access to food and water.

Both male and female mice were randomly assigned to the following seven groups, each consisting of four animals: vehicle control (VC), high Muse cell MSC low-dose (1×10⁶ cells/mouse), high Muse cell MSC medium-dose (5×10⁶ cells/mouse), high Muse cell MSC high-dose (10×10⁶ cells/mouse), high Muse cell MSC exosomes low-dose (10×10⁹/mouse), high Muse cell MSC exosomes medium-dose (50×10⁹/mouse), high Muse cell MSC exosomes high-dose (100×10⁹/mouse). Mice in the VC group were administered PBS.

### High Muse Cell MSC or High Muse Cell Exosome Administration

Each mouse received a single bolus of the dispersion via intraperitoneal injection. The shots were administered using a sterile hypodermic syringe and a stainless-steel needle (26 G). The dosage was 10 mL/kg body weight and was adjusted based on the animal's body weight on the day of treatment.

### Mortality and Clinical Signs

During the first four hours following injection, all animals were monitored for mortality (at intervals of 30 min, 1 h, 2 h, and 4 h), and then once a day for the next 14 days. In the same way, all animals were monitored for indications of toxicity (at intervals of 30 min, 1 h, 2 h, and 4 h), and then once a day for the next 14 days. The presence of any symptoms, their progression or disappearance, if any, would be documented.

### Body Weight

The body weights were measured the day before treatment, and on days 1, 3, 11 and 15 (just before sacrifice). The change in body weight of the individual animals with respect to the initial measurement and group mean values were calculated.

### Hematology

Blood sampling for hematology was performed from the tail vein on days 2 and 15 and sodium citrate was used as an anticoagulant. The following parameters were analyzed using a hematological autoanalyzer according to manufacturer's instructions (ADVIA2120i Hematology analyzer, Bayer, USA): white blood cell count (WBC), red blood cell count (RBC), hemoglobin concentration (Hb), hematocrit (Hct), platelets (PLT), neutrophils (NEU), lymphocytes (LYM), monocytes (MONO), eosinophils (EOS), basophils (BASO).

### Serum Biochemistry

Serum was isolated and used to determine the levels of alanine aminotransferase (ALAT), aspartate aminotransferase (ASAT), and total protein. Measurements were carried out using a clinical chemistry autoanalyzer (200FR NEO, Toshiba Co., Japan).

### Necropsy

After the study period, on day 15, the animals were humanely sacrificed and subjected to necropsy. The gross pathological changes were documented by examination of lung, kidney, spleen and liver samples under a microscope.

### Statistical Analysis

Experimental data were processed using the Statistica 10.0 software package. Data are shown in graphs as median ranges. For intergroup comparison, the nonparametric Kruskal-Wallis's test was used, with adjustment for multiple comparisons (Rosner et al., 2006). For paired comparison (before and after treatment inside the same group), the nonparametric Wilcoxon test was used (Ostertagová et al., 2014). For both comparisons, the differences were considered statistically significant with a P-value equal to or less than 0.05.

### Results

The trial was carried out with up to 10×10⁸ high Muse cell MSCs per mouse and 100×10⁹ exosome particles per mouse. These doses are ten times what would be given to humans as a therapeutic dose and as a result, are thought to provide an appropriate safety margin given the proposed therapeutic use [14], [15]. Because 10×10⁸ MSC/mouse and 100×10⁹ exosomes/mouse did not result in any health issues in the treated mice, additional doses were not tested. The lowest lethal dose, maximum acceptable dose, and median lethal dose of MSCs from umbilical cord tissue and their exosomes following acute intraperitoneal injection in mice could not be determined in our study because there were no adverse effects or death among the treated mice in this study. Likewise, the LD50 of Whartons jelly high Muse cell MSCs and their exosomes for intraperitoneal administration in mice is also expected to be more than 10×10⁶ MSC/mouse and 100×10⁹ exosomes/mouse. Taken together, the results suggest that the MSCs and their derived exosomes were safe and did not produce adverse effects.

### Clinical Signs and Mortality

On the day of intraperitoneal treatment dosing and regularly thereafter until the 14th day, frequent clinical examinations at intervals of 30 min, 1 h, 2 h, and 4 h did not reveal any unusual clinical features or mortalities among the treated mice (Table 7).

TABLE 7
Summary of Mortality

| | | Incidence of mortality | |
|---|---|---|---|
| | | (pooled male and female) | |
| Group | Treatment | Absolute | % Mortality |
| Vehicle control | 0.2 ml of PBS I.P. | 0/4 | 0 |
| High Muse Cell MSC low-dose | 1 × 10⁶ cells/mouse I.P. | 0/4 | 0 |
| High Muse Cell MSC medium | 5 × 10⁶ cells/mouse I.P. | 0/4 | 0 |
| dose | | | |
| High Muse Cell MSC high-dos | 10 × 10⁶ cells/mouse I.P. | 0/4 | 0 |
| High Muse Cell MSCExo low- | 10 × 10⁹/mouse I.P. | 0/4 | 0 |
| dose | | | |
| High Muse Cell MSCExo | 50 × 10⁹/mouse I.P. | 0/4 | 0 |
| medium-dose | | | |
| High Muse Cell MSCExo high | 100 × 10⁸/mouse I.P. | 0/4 | 0 |
| dose | | | |

Absolute mortality is presented as the number of animals that died/numbers treated.

### Body Weight

The body weights of the treated animals irrespective of gender were not adversely affected during the entire period of investigation, including and up to the time they were humanely euthanized on day 15.

### Blood Parameters

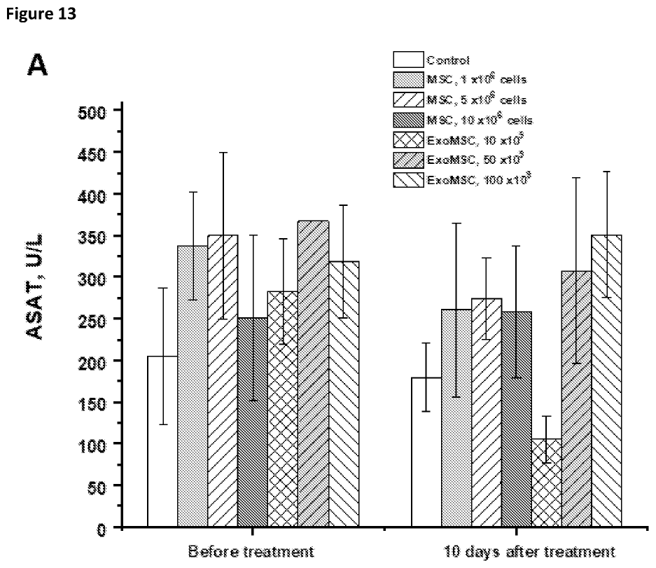

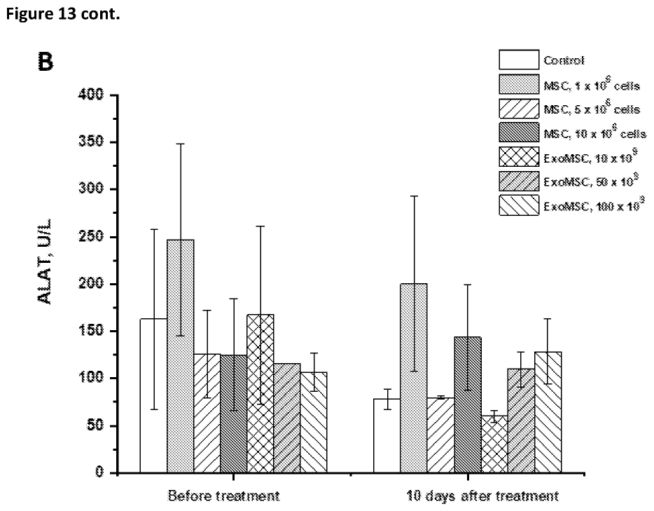

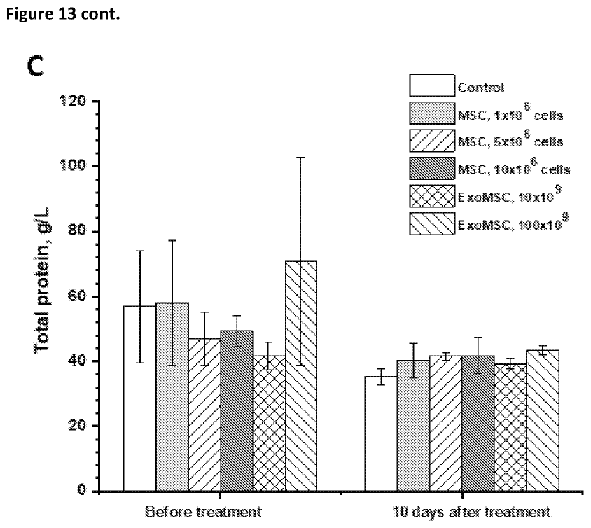

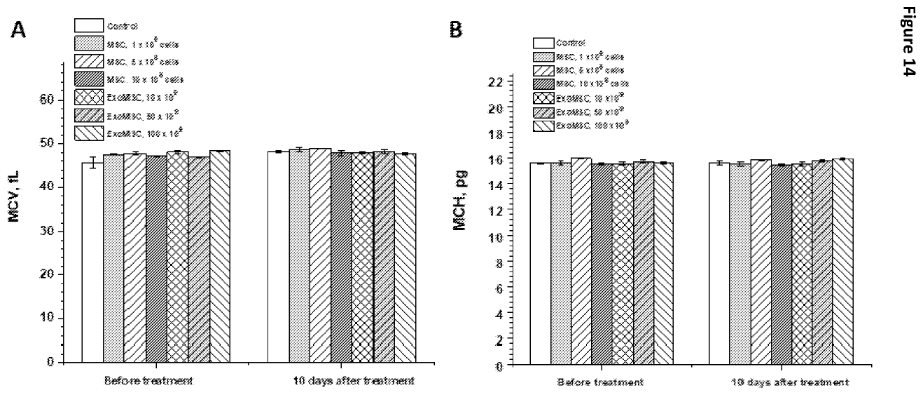

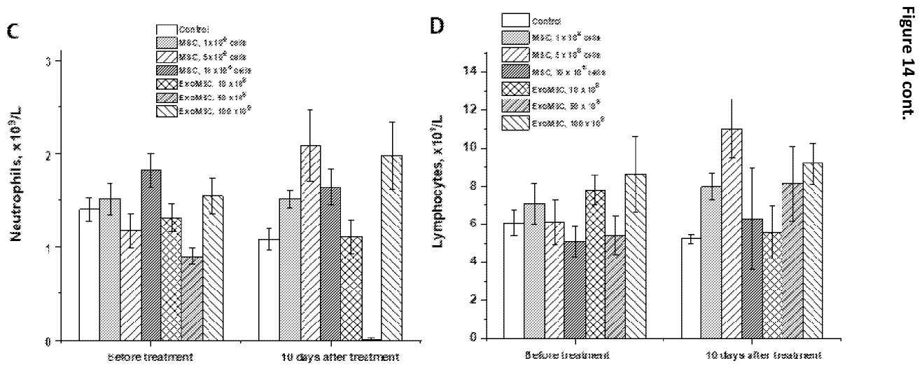

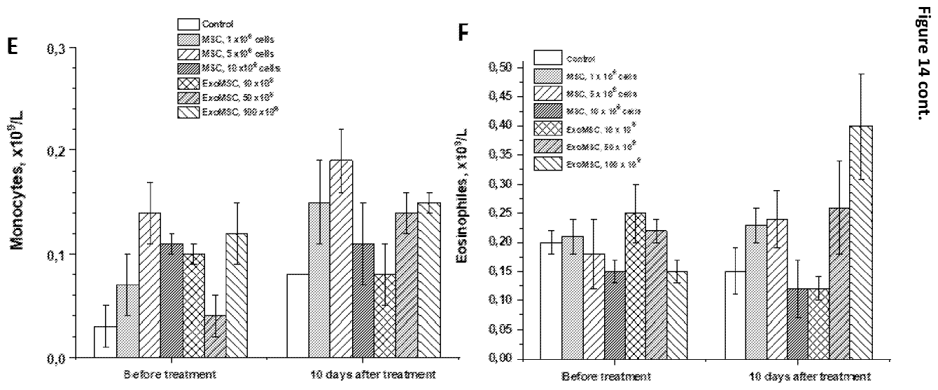

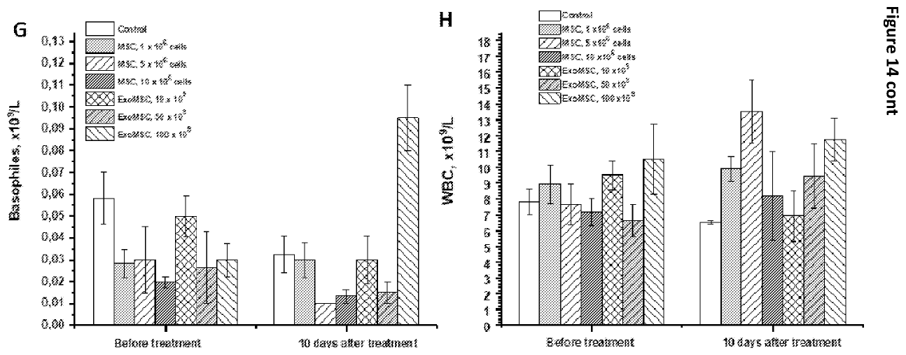

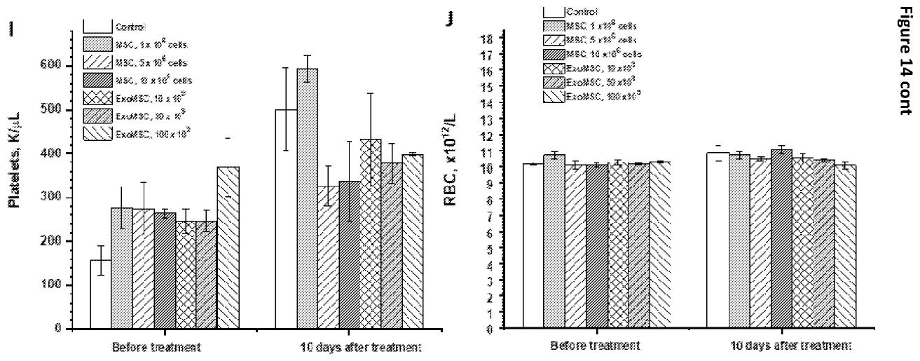

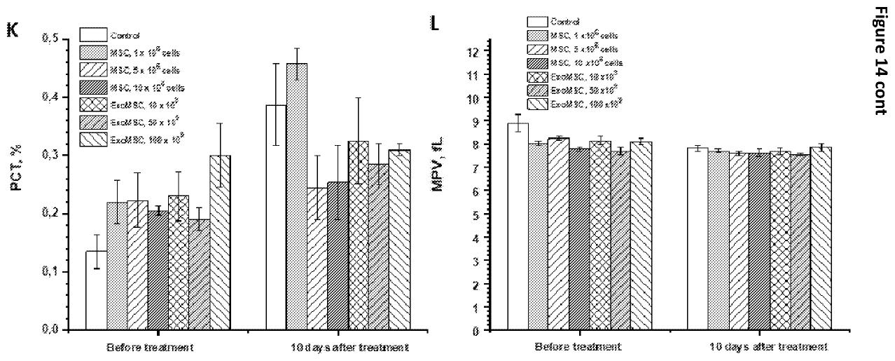

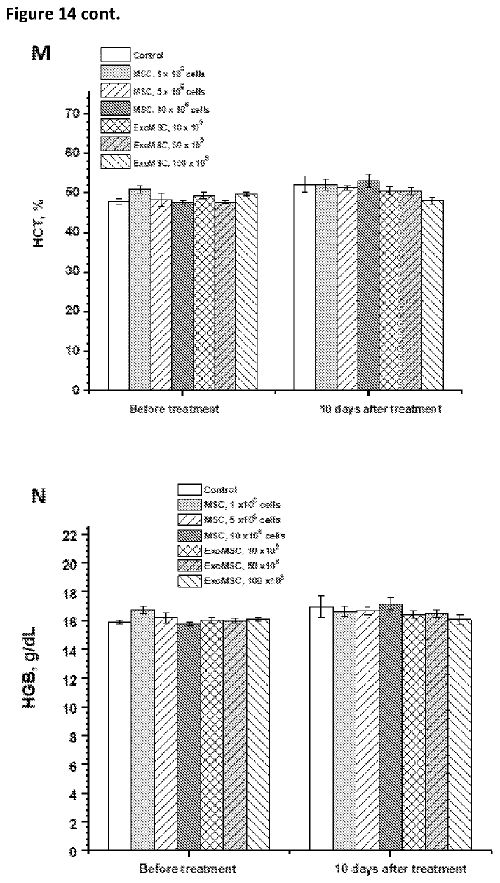

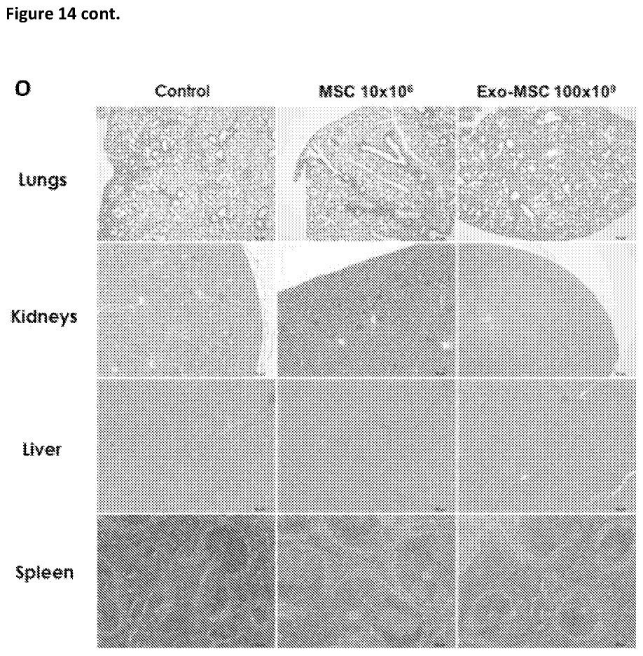

Hematological and biochemical evaluations showed no statistically significant differences in any laboratory parameters between their states before and after cell or exosome administration, as well as between control and treatment groups (Tables 8-11; see also FIGS. 13 and 14 for corresponding bar graphs for each marker).

TABLE 8
Counts of white blood cells (WBC), neutrophiles (NEU), lymphocytes
(LYM), monocytes (MONO) and eosinophiles (EOS) before and after
injection of tested treatments. Data shown as median ranges

| | WBC, 10⁹/L | | NEU, 10⁹/L | | LYM, 10⁹/L |
|---|---|---|---|---|---|
| Groups | before | after | before | after | before |
| control | 7.04 | 6.64 | 1.37 | 1.10 | 5.43 |
| | (5.88:11.47) | (6.32:6.71) | (1.14:1.97) | (0.80:1.30) | (4.47:9.0) |
| High Muse Cell | 7.55 | 10.11 | 1.60 | 1.49 | 5.95 |
| MSC, 1 × 10⁶ | (6.36:14.24) | (7.95:11.40) | (0.9:2.0) | (1.34:1.8) | (5.03:11.82) |
| High Muse Cell | 8.14 | 13.55 | 1.18 | 2.08 | 6.35 |
| MSC, 5 × 10⁶ | (5.22:9.54) | (11.55:15.55) | (0.85:1.47) | (1.69:2.46) | (3.99:8.02) |
| High Muse Cell | 6.20 | 9.44 | 1.74 | 1.68 | 4.33 |
| MSC, 1 × 10⁷ | (5.49:11.04) | (2.84:12.25) | (1.30:2.34) | (1.3-1.95) | (3.43:8.4) |
| High Muse Cell | 8.74 | 6.52 | 1.25 | 1.11 | 6.99 |
| MSCExo, 10 × 10⁹ | (7.69:13.66) | (4.16:10.47) | (0.85:1.91) | (0.78:1.56) | (6.11:11.14) |
| High Muse Cell | 5.72 | 9.44 | 0.99 | 0.91 | 4.72 |
| MSCExo, 50 × 10⁹ | (5.44:8.70) | (7.39:11.49) | (0.71:0.99) | (.89:0.92) | (4.12:7.4) |
| High Muse Cell | 10.79 | 11.73 | 1.5 | 1.92 | 8.59 |
| MSCExo, 100 × 10⁹ | (4.85:16.74) | (10.38:13.07) | (0.97:2.16) | (1.56:2.27) | (3.7:14.09) |

| | LYM, 10⁹/L | MONO, 10⁹/L | | EOS, 10⁹/L | |
|---|---|---|---|---|---|
| Groups | after | before | after | before | after |
| control | 5.18 | 0.03 | 0.08 | 0.2 | 0.13 |
| | (4.77:5.74) | (0:0.1) | (0.07:0.09) | (0.12:0.27) | (0.07:0.26) |
| High Muse Cell | 8.01 | 0.06 | 0.12 | 0.2 | 0.23 |
| MSC, 1 × 10⁶ | (6.31:9.57) | (0:0.16) | (0.08:0.27) | (0.13:0.31) | (0.17:0.29) |
| High Muse Cell | 11.04 | 0.15 | 0.19 | 0.15 | 0.24 |
| MSC, 5 × 10⁶ | (9.50:12.58) | (0.08:0.18) | (0.16:0.22) | (0.1:0.29) | (0.19:0.28) |
| High Muse Cell | 7.81 | 0.11 | 0.15 | 0.14 | 0.16 |
| MSC, 1 × 10⁷ | (1.09:9.96) | (0.08:0.13) | (0.03:0.16) | (0.10:0.21) | (0.03:0.17) |
| High Muse Cell | 5.27 | 0.11 | 0.08 | 0.22 | 0.13 |
| MSCExo, 10 × 10⁹ | (3.2:8.56) | (0.06:0.12) | (0.03:0.14) | (0.12:0.46) | (0.07:0.15) |
| High Muse Cell | 8.13 | 0.05 | 0.14 | 0.21 | 0.26 |
| MSCExo, 50 × 10⁹ | (6.19:10.06) | (0:0.06) | (0.11:0.16) | (0.18:0.26) | (0.18:0.33) |
| High Muse Cell | 9.17 | 0.11 | 0.15 | 0.14 | 0.4 |
| MSCExo, 100 × 10⁹ | (8.09:10.25) | (0.05:0.20) | (0.14:0.16) | (0.1:0.21) | (0.3:0.49) |

TABLE 9
Counts of basophiles (BASO) and platelets (PLT), mean platelet volume (MPV) and plateletcrit
(PCT) before and after injection of tested treatments. Data shown as medians (ranges)

| | BASO, 10⁹/L | | PLT, K/μL | | MPV, fL | | PCT, % | |
|---|---|---|---|---|---|---|---|---|
| Groups | before | after | before | after | before | after | before | after |
| control | 0.06 | 0.04 | 185 | 529 | 8.6 | 7.9 | 0.15 | 0.41 |
| | (0.02:0.09) | (0.01:0.05) | (104:267) | (248:698) | (8.0:10.6) | (7.5:8.0) | (0.10:0.23) | (0.20:0.56) |
| High Muse Cell | 0.03 | 0.03 | 282 | 587 | 8.0 | 7.7 | 0.22 | 0.45 |
| MSC, 1 × 10⁶ | (0.01:0.05) | (0.01:0.05) | (120:456) | 524:673 | (7.7:8.4) | (7.6:79) | (0.10:0.36) | (0.40:0.53) |
| High Muse Cell | 0.02 | 0.01 | 281 | 326 | 8.2 | 7.6 | 0.23 | 0.25 |
| MSC, 5 × 10⁶ | (0.01:0.06) | (0.01:0.01) | (166:376) | (249:403) | (8.1:8.4) | (7.5:7.7) | (0.14:0.30) | (0.19:0.3) |
| High Muse Cell | 0.02 | 0.01 | 263 | 287 | 7.8 | 7.5 | 0.21 | 0.19 |
| MSC, 1 × 10⁷ | (0.01:0.03) | (0.01:0.02) | (229:305) | (212:511) | (7.6:8.1) | (7.4:7.9) | (0.18:0.23) | (0.17:0.38) |
| HighMuse Cell | 0.06 | 0.03 | 328 | 379 | 8.1 | 7.7 | 0.19 | 0.30 |
| MSCExo, 10 × 10⁹ | (0.01:0.07) | (0.01:0.06) | (178:354) | (233:732) | (7.6:8.9) | (7.3:8.0) | (0.16:0.42) | (0.18:0.53) |
| High Muse Cell | 0.01 | 0.02 | 229 | 377 | 7.6 | 7.6 | 0.17 | 0.29 |
| MSCExo, 50 × 10⁹ | (0.01:0.06) | (0.01:0.02) | (217:297) | (322:422) | (7.5:8.0) | (7.5:7.6) | (0.17:0.23) | (0.25:0.32) |
| High Muse Cell | 0.03 | 0.09* | 348 | 398 | 8.2 | 7.9 | 0.28 | 0.31 |
| MSCExo 100 × 10⁹ | (0.01:0.05) | (0.08:0.11) | (206:644) | (394:402) | (7.7:8.5) | (7.7:8.0) | (0.18:0.53) | (0.30:0.32) |

TABLE 10
Count of erythrocytes (red blood cells, RBC), hematocrit (HCT), hemoglobin (HGB) and mean corpuscular volume
or erythrocytes (MCV) before and after injection of tested treatments. Data shown as medians (ranges)

| | RBC, 10¹²/L | | HCT, % | | HGB, g/dL | | MCV, IL | |
|---|---|---|---|---|---|---|---|---|
| Groups | before | after | before | after | before | after | before | after |
| control | 10.18 | 10.69 | 48.1 | 51.4 | 15.9 | 16.5 | 47.1 | 48.3 |
| | (9.91:10.34) | (9.96:12.04) | (45.5:49.3) | (48.3:57.9) | (15.4:16.3) | (15.8:19.1) | (45.9:47.7) | (47.8:48.5) |
| High Muse Cell | 10.07 | 10.82 | 50.9 | 52.3 | 16.6 | 16.8 | 47.5 | 48.6 |
| MSC, 1 × 10⁶ | (10.04:11.42) | (10.14:11.05) | (48.4:54.4) | (49.1:55.1) | (16.0:17.9) | (15.6:17.4) | (47.1:48.2) | (47.5:50) |
| High Muse Cell | 10.01 | 10.52 | 47.5 | 51.4 | 16.0 | 16.7 | 47.5 | 48.9 |
| MSC, 5 × 10⁶ | (9.74:10.62) | (10.41:10.62) | (46.1:51.6) | (50.8:51.9) | (15.7:16.9) | (16.4:16.9) | (47.3:48.6) | (48.8:48.9) |
| High Muse Cell | 10.04 | 10.83 | 47.3 | 52 | 15.7 | 16.8 | 47.2 | 48.1 |
| MSC, 1 × 10⁷ | (9.83:10.43) | (10.81:11.54) | (46.3:49.2) | (50.7:56.2) | (15.4:16.1) | (16.6:18.0) | (26.7:47.5) | (46.8:48.7) |
| High Muse Cell | 10.27 | 10.75 | 49.8 | 51.5 | 16.1 | 16.6 | 47.9 | 48.0 |
| MSCExo, 10 × 10⁹ | (9.69:10.84) | (9.77:10.89) | (46.0:51.9) | (47.2:51.2) | (15.1:16.6) | (15.6:16.8) | (47.5:49.5) | (47.7:48.3) |
| High Muse Cell | 10.15 | 10.44 | 47.4 | 50.4 | 15.9 | 16.5 | 47.0 | 48.3 |
| MSCExo, 50 × 10⁹ | (10.05:10.26) | (10.35:10.53) | (47.2:48.4) | (49.5:51.3) | (15.7:16.3) | (16.2:16.7) | (46.7:47.2) | (47.8:48.7) |
| High Muse Cell | 10.25 | 10.07 | 49.6 | 48.1 | 16.2 | 16.1 | 48.3 | 47.8 |
| MSCExo 100 × 10⁹ | (10.07:10.62) | (9.84:10.3) | (48.3:51.4) | (47.2:48.9) | (15.6:16.4) | (15.7:16.4) | (48.0:49.0) | (47.5:48.0) |

TABLE 11
Biochemical parameters of mice blood: levels of alanine transaminase (ALAT), aspartate transaminase (ASAT)
and total protein before and after injection of tested treatments. Data shown as medians (ranges)

| | ALAT, U/l | | ASAT, U/l | | TOTAL PROTEIN, g/L | |
|---|---|---|---|---|---|---|
| Groups | before | after | before | after | before | after |
| control | 71.9 | 72.2 | 128.5 | 165.4 | 41.7 | 37.6 |
| | (56.3:450.0) | (53.2:105.5) | (113.4:450.0) | (98.6:288.2) | (35.6:108.2) | (28.1:37.9) |
| High Muse Cell | 235.3 | 184.8 | 338.9 | 261.9 | 40.7 | 36.0 |
| MSC, 1 × 10⁶ | (65.3:450.0) | (41.2:390.7) | (222.0:450.0) | (67.7:450.0) | (35.5:115.4) | (33.2:55.9) |
| High Muse Cell | 126.0 | 80.0 | 349.8 | 274.2 | 46.8 | 41.6 |
| MSC, 5 × 10⁶ | (80.0:172.0) | (78.2:81.7) | (249.6:450.0) | (225.4:332.9) | (38.7:55.0) | (40.4:42.9) |
| High Muse Cell | 78.4 | 75.8 | 163.5 | 157.7 | 53.3 | 38.2 |
| MSC, 1 × 10⁷ | (53.6:243.2) | (49.5:354.0) | (141.2:450.0) | (109.8:450.0) | (39.6:55.0) | (32.9:57.6) |
| High Muse Cell | 75.2 | 57.6 | 264.1 | 81.8 | 40.0 | 39.3 |
| MSCExo, 10 × 10⁹ | (68.2:450.0) | (48.0:75.8) | (152.8:450.0) | (69.3:187.2) | (32.9:57.6) | (35.6:43.1) |
| High Muse Cell | 107.9 | 108.0 | 334.9 | 410.4 | 40.6 | 44.5 |
| MSCExo, 100 × 10⁹ | (55.3:154.1) | (71.9:225.9) | (156.2:450.0) | (132.8:450) | (36.9:134.9) | (39.7:44.97) |

### Histopathological Evaluation

FIG. 140 shows representative images of the H&E-stained tissues of mice lung, kidney, liver and spleen. Typical normal structures of different tissues were visible in both the control and high-dose high Muse MSC and high Muse MSC-Exo treatment groups. Histopathological evaluations did not detect inflammation or necrosis in the treatment groups. Overall, all harvested tissues were normal, and the histopathological findings excluded the occurrence of any damage or toxicity.

### Example 14: Anti-Inflammatory Effects Using Carrageenan-Induced Paw Edema Model

In order to assess the anti-inflammatory effect of the Muse cell derived EVs, various EV fractions were analyzed for anti-inflammatory efficacy using the paw edema model [16].

### 1. Induction of Paw Edema

In our initial experiment, fifty-five female C57BL/6 mice (Charles River, Eastern Europe) were randomly assigned to one of five treatment groups:
(1) (−) Control (PBS)
   (2) (+) Control (Dexamethasone: 20-30 mg/kg)
   (3) EV100 (1, 10, or 100 billion EVs)
   (4) EV500 (1, 10, or 100 billion EVs)
   (5) Muse cell enriched culture (100K, 1 million, or 10 million cells)

Paw edema was induced in each animal by intra-plantar injection of 20 μl of a 1% (w/v) aqueous Carrageenan solution (SIGMA Aldrich) into the right hind paw. The carrageenan injection triggers an acute inflammatory response which is accompanied by edema formation. It is well known that the severity of the edema is directly proportional to the level of inflammation. An anti-inflammatory response can thus easily be attributed to agents that result in a reduction of the edema severity, i.e. thickness of the edemic paw.

### 2. Administration of Treatment or Control Agent

Thirty minutes after the carrageenan injection, each animal received one dose of the control agent (treatment groups 1-2) or experimental agents (treatment groups 3-5) according to the respective assigned treatment group. The agent was administered by intra peritoneal (i.p.) injection. PBS was also used to resuspend EV100 and EV500 samples.

### 3. Assessment of Edema Development

a. Single Dose

The intensity of the edema development and/or the amelioration thereof was assessed by measuring the paw width (the distance from one outer finger to the opposite outer finger) or paw thickness of the treated paw prior to treatment administration, and 2 h, 24 h, 48 h, and 72 h after administration of the control and experimental agents.

The inflammatory reaction induced by carrageenan, developed a form of swelling/edema with an increase in the paw thickness. The edema thickness of the animals treated with PBS remained relatively stable over an extended period of time.

i. Immediate Response

All tested EV concentrations exhibited an anti-inflammatory effect (data not shown). From these experiments, the most promising doses were chosen to repeat the experiments. The medium concentration of EV100, and the Muse cell enriched cellular fraction (hereinafter Muse cells) are shown in FIG. 5, wherein the immediate response 1.5 hours after administration of the therapeutic agents is shown. Both EV100 and the Muse cells show a reduction in the paw thickness relative to the saline control as well as the dexamethasone control. The glucocorticoid dexamethasone showed no effect at this time point, which is agreement with the known pharmacokinetics of dexamethasone, which is too slow to affect edema formation.

ii. Early Response

After 1 day of administration of the therapeutic agents, the effect of dexamethasone is detectable, clearly showing a marked reduction of paw thickness (FIG. 6). EV100 and Muse cells further inhibited the inflammatory response, resulting in a further reduction of the edema. The degree of the anti-inflammatory effect of dexamethasone, EV100 and Muse cells was comparable.

iii. Intermediate Response

After about 1 week of administration of the therapeutic agents, the anti-inflammatory effects of dexamethasone, EV100 and Muse cells were further increased, as demonstrated by a further reduction in paw thickness relative to the PBS treated control.

iv. Late Response

After about 2 weeks of administration of the therapeutic agents, the anti-inflammatory effects of dexamethasone had waned off, and the degree of the inflammation was comparable to the carrageenan injected paw treated with PBS (FIG. 8). Surprisingly, the anti-inflammatory effect of EV100 and Muse cells was still present even after two weeks of injection.

b. Multiple Doses

In order to assess whether the effect could be further improved, the experiments were repeated as indicated above, wherein dexamethasone and EV100 were compared, and injected not only 0.5 hours after carrageenan administration, but also at 1 day, 2 day, 3 day and 4 day post administration of carrageenan. The change in paw thickness is shown at 1 day, 6 days and 11 days after administration of the last dose (FIGS. 9-11).

After 1 day of repeated administration of the therapeutic agents, the reduction in paw thickness, i.e. inflammation, was evident in dexamethasone and EV100 injected animals, wherein the anti-inflammatory effect was a little more pronounced in the dexamethasone treated animals (FIG. 9).

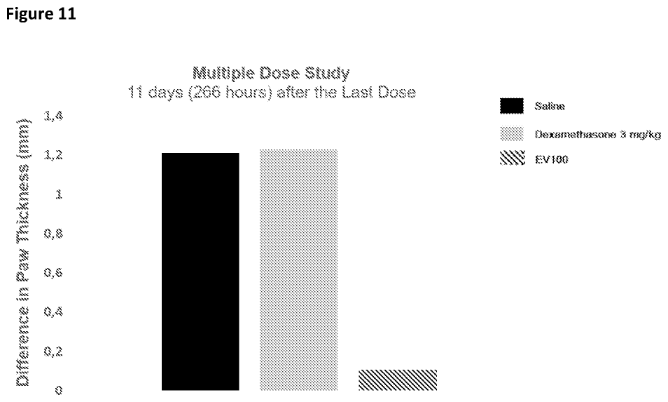

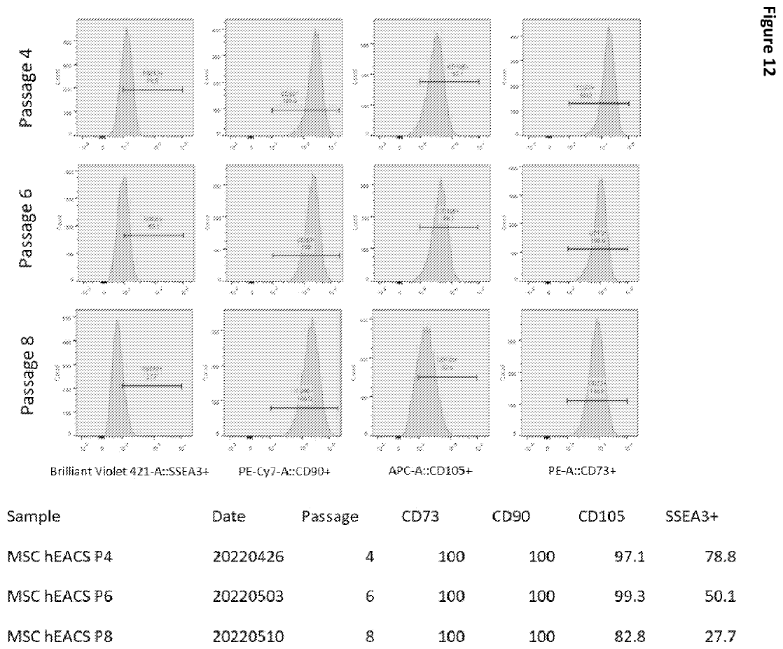

Six days (146 hours) after administration of the last dose, the animals repeatedly injected with EV100 showed the strongest reduction in paw thickness (FIG. 10). This effect was even more pronounced 11 days (266 hours) after the last administration. At this time point (FIG. 11), dexamethasone treated animals and saline treated animals were indistinguishable from each other, suggesting the effect of dexamethasone had expired. In contrast, the anti-inflammatory effect of EV100 remained and was able to suppress inflammation to an extent that nearly abolished any difference in paw thickness relative to the uninjected baseline thickness (FIG. 11).

These experiments suggest that EVs exert a potent anti-inflammatory effect when delivered systemically, over a long period of time, that exceeds the effect of corticosteroids, such as dexamethasone. Further experiments are underway to measure a panel of cytokines in serum samples of the mice before, during, and after treatments using Luminex analysis. This will provide a functional readout for the anti-inflammatory effects of EVs in vivo.

### Example 15: Anti-Inflammatory Effect Using LPS-Induced Sepsis Rodent Model

Administration of LPS (endotoxin), an outer membrane component of Gram-negative bacteria, can induce systemic inflammation in mice. This model is used to mimic the initial clinical features of sepsis, including extensive pro-inflammatory cytokine production.

### Sepsis Induction

A preliminary study was performed to evaluate the acute inflammatory changes in mice at different times after sepsis induction. Twenty male BALB/C mice aged 6 to 8 weeks were prepared from the National Science and Research Institute and Medicine Innovative Center, Vilnius, Lithuania. The animals were kept under standard vivarium conditions with a 12-h light/dark cycle, a complete diet, and free access to food and water. Animal care and handling were carried out under national and international requirements for the treatment of animals used for scientific purposes.

Induction of acute systemic inflammation in mice was performed at 0 h by intraperitoneal injection (I.P.) of 10 mg/kg of LPS derived from Escherichia coli (Escherichia coli 0111:B4, Sigma), a classical inflammatory bacterial endotoxin. For each mouse, the LPS dosage was adjusted according to the body weight and dissolved in 0.2 ml of sterile PBS and injected at room temperature.

Different time points have been considered as criteria for sacrificing mice and evaluating their inflammatory status for this experiment as well as for further investigation. To this end, the mice were randomly divided into a healthy group (5 mice) and an LPS-treated group (15 mice). Mice from the healthy group were left intact and were all sacrificed at 0 h. The mice in the LPS-treated group were sacrificed and assessed five times (three mice at each time point): 2 h, 4 h, 6 h, 8 h, and 24 h after LPS model induction. The severity of clinical signs of induced systemic inflammation was scored using the Murine Sepsis Score (MSS). Alterations in peripheral blood cell counts and biochemistry parameters, and cytokine levels in blood plasma and lung tissue were also assessed. Hematoxylin and eosin staining was performed on lung samples to determine an injury score and to compare alveolar wall thickness and alveolar spaces.

Data achieved in this experiment was used to choose time points of the most severe LPS-induced manifestations, including changes in cytokines levels and lung tissue damage (data not shown). The 6 hour and 24 h time point after LPS induction were chosen to further test the anti-inflammatory efficacy MSC-derived exosomes (Exo-MSC).

Treatment with High Muse Cell MSC-Derived Exosomes

Next, in order to investigate whether LPS-induced inflammatory changes are altered in mice treated systemically with high Muse cell MSC exosomes (“Exo”), Male BALB/C mice at 6-weeks of age (n=24) were subjected to intraperitoneal injection of 10 mg/kg of LPS. Animals were divided into the following groups:
(1) untreated group (LPS+PBS),
(2) dexamethasone-treated group (LPS+Dex), and
(3) exosome-treated group (LPS+Exo).

For the exosome-treated group, high Muse cell MSC-derived exosomes (1×10⁹ particles) were injected I.P. into mice at 48 and 24 h before and 1 h after LPS administration. The exosomes were derived from high Muse cell MSC obtained from Wharton's jelly according to the method of the invention and were 63% Muse as determined by SSEA3+ FACS analysis.

Treatment with highly potent anti-inflammatory glucocorticoid dexamethasone (3 mg/kg) was given to mice I.P. only once at 1 h post-LPS challenge. Based on the biochemistry and cytokine results of the first experiment above, two-time points (6 h and 24 h) were chosen for sacrifice and measurements (3 animals per group per timepoint) to evaluate and compare the protective effects of DEX, and exosomes in experimental groups.

To assess the impact of tested treatments on healthy animals, 6 intact male BALB/C mice (6 weeks old, 3 mice per treatment) were pretreated with exosomes (1×10⁹ particles) 48 and 24 h before sacrifice.

### MSS Scoring

The severity of inflammatory manifestations in the sepsis mice models was scored according to the Murine Sepsis Score (MSS) system (17. This system examines seven major factors, graded from 0 to 4. The MSS number is obtained from the sum of these scores, which indicates the severity of sepsis (Table 12)

TABLE 12
Scoring sepsis severity with MSS system

| Factor | Definition | Score |
|---|---|---|
| Appearance | The fur cover is smooth. | 0 |
| | Parts of body hair are skewered. | 1 |
| | Most of the hair on the back of the body is skewered. | 2 |
| | Fur skewers may or may not be known; Mice have a puffy appearance. | 3 |
| | Fur skewers may or may not be known; The mouse looks weak and weak. | 4 |
| Level of | The mouse is active. | 0 |
| consciousness | The mouse is active but avoids standing up straight. | 1 |
| | Mouse activity is significantly slowed. The mouse is still moving. | 2 |
| | Mouse activity is impaired. The mouse moves only with stimulation and its | 3 |
| | movements vibrate. | 4 |
| | Mouse activity is severely impaired. Mice remain stationary when stimulated | |
| Activity | The activity is normal. The mouse is eating, drinking, climbing, running, and | 0 |
| | fighting activities. 0 | 1 |
| | The activity is slightly suppressed. The mouse moves on the floor of the | 2 |
| | cage. 1 | 3 |
| | The activity is suppressed. The mouse is stationary with occasional search | 4 |
| | movements. 2 | |
| | The mouse is not active. The mouse is stationary. 3 | |
| | The mouse is not active. There are tremors, especially in the hind leg | |
| Response to | The mouse responds immediately to a tactile or sound stimulus. | 0 |
| the stimulus | The response to the vocal stimulus is slow or absent. Mouse responds | 1 |
| | strongly to tactile stimuli. | 2 |
| | There is no response to the vocal stimulus. The response to the tactile | 3 |
| | stimulus is moderate. | |
| | There is no response to the vocal stimulus. The response to the tactile | 4 |
| | stimulus is low. | |
| | There is no response to the vocal stimulus. The response to the tactile | |
| | stimulus is weak or absent. If pushed, it cannot keep itself straight. | |
| Eyes | Eyes open. | 0 |
| | The eyes are not completely open. There may be discharge. | 1 |
| | The eyes are at least half closed. There may be discharge. | 2 |
| | Eyes are half or more closed. There may be discharge. | 3 |
| | The eyes are closed or have a milky discharge | 4 |
| Number of | Normal, rapid breathing of mice. | 0 |
| breaths | Respiration is slightly reduced (respiration rate cannot be measured with the eye). | 1 |
| | Respiration is relatively reduced (respiration rate is the highest rate that can | 2 |
| | be measured with the eyes). | |
| | Breathing is greatly reduced. Respiration rate can be easily measured with | 3 |
| | the naked eye. The interval between breaths is 0.5 s. | 4 |
| | Respiration is greatly reduced (the interval between breaths is >1 s). | |
| Respiratory | Normal | 0 |
| quality | Short periods of heavy and laborious breathing | 1 |
| | Heavy and laborious breathing, without shortness of breath | 2 |
| | Heavy and laborious breathing, with intermittent shortness of breath | 3 |
| | Shortness of breath with open mouth | 4 |

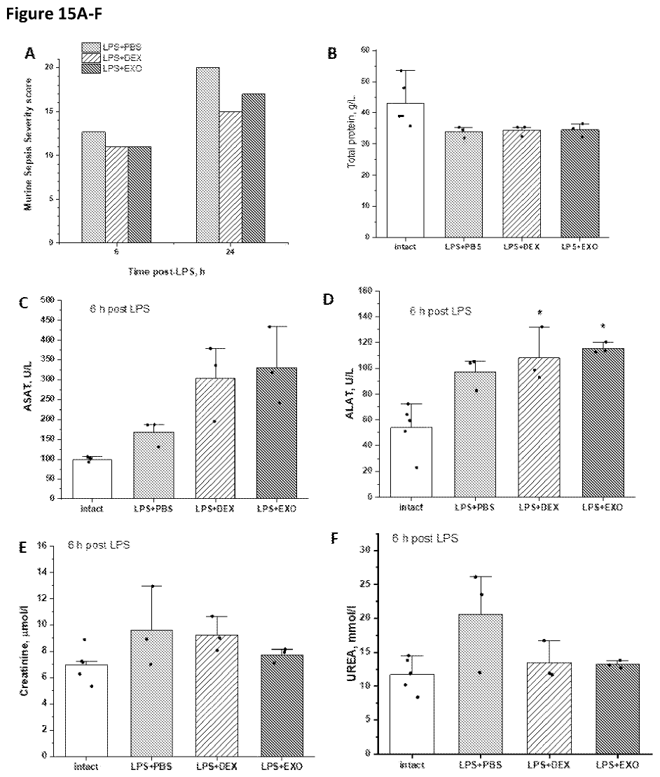

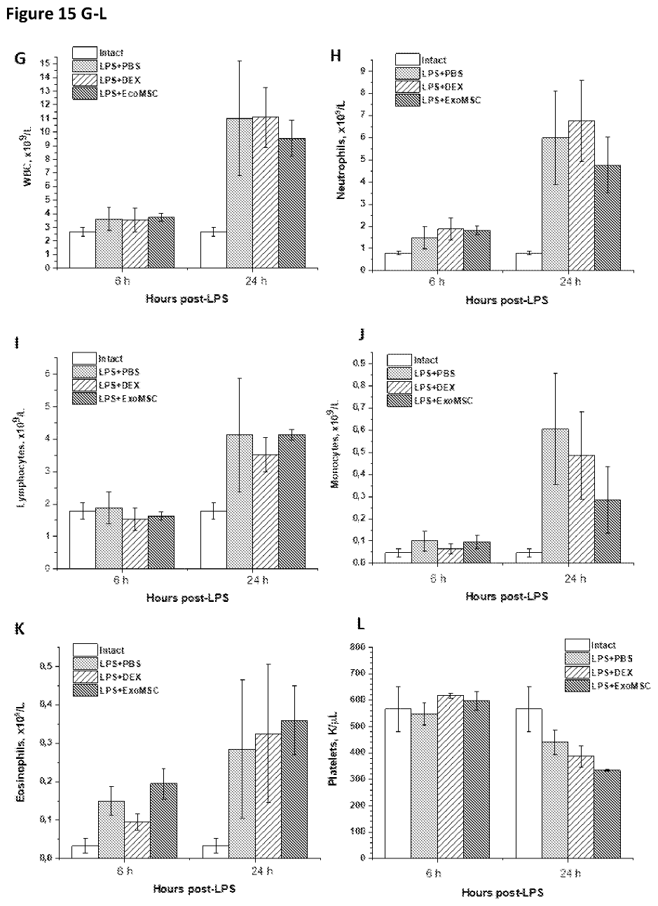

FIG. 15A shows that at 6 h post LPS, MSS scores were similar in all groups; however, at 24 h post LPS, both the Dex group (2) and Exo group (3) scored lower than the PBS control group (1). Their fur appeared similar and less skewered than in the PBS-treated mice. Their level of activity was reduced, but they were still reacting and moving when they were touched or handled. Their eyes were more open and more responsive compared to the PBS mice, where the eyes were at least half closed and showing little movement. This suggests that the sepsis phenotype was less pronounced in the dex and exosome-treated groups.

### Blood Biochemistry

Mice were anesthetized by intramuscular injection of 80 mg/kg ketamine and 5 mg/kg xylazine mixture. Serum was isolated from their heart blood by centrifuging at 3000 g for 7 min. The biochemical autoanalyzer (200FR NEO, Toshiba Co., Japan) measured the serum levels of aspartate aminotransferase (ASAT) and alanine transaminase (ALAT) enzymes to evaluate the liver function as well as urea and creatinine to examine the renal function (FIG. 15 C-F). At 6 h post LPS treatment, except for ALAT, all biochemical evaluations showed no statistically significant differences in laboratory parameters.

### Hematology

Blood sampling for hematology was performed from the heart puncture. The following parameters were analyzed: white blood cell count (WBC), platelets (PLT), neutrophils (NEU), lymphocytes (LYM), monocytes (MONO), and eosinophils (EOS) (FIG. 15 G-L). This was done using a hematological autoanalyzer (ADVIA2120i Hematology analyzer, Bayer, USA). There were no differences in hematological parameters between the experimental groups. All parameters except for platelets, were increased in the LPS treated groups (1)-(3) at 24 h after LPS induction, confirming inflammation (FIG. 15 G-L).

### Cytokine Measurements

Pro- and anti-inflammatory cytokines (IL-1β, IL-6, IL-10, and IFN-γ) were measured in plasma serum samples with a commercially available ProcartaPlex™ Multiplex Immunoassay Kit (Thermo Fisher Scientific Inc., Waltham, Massachusetts, USA) using Luminex™ magnetic bead technology according to the instructions issued by the manufacturer. IL-1β, and IL-10 were additionally measured in lung samples.

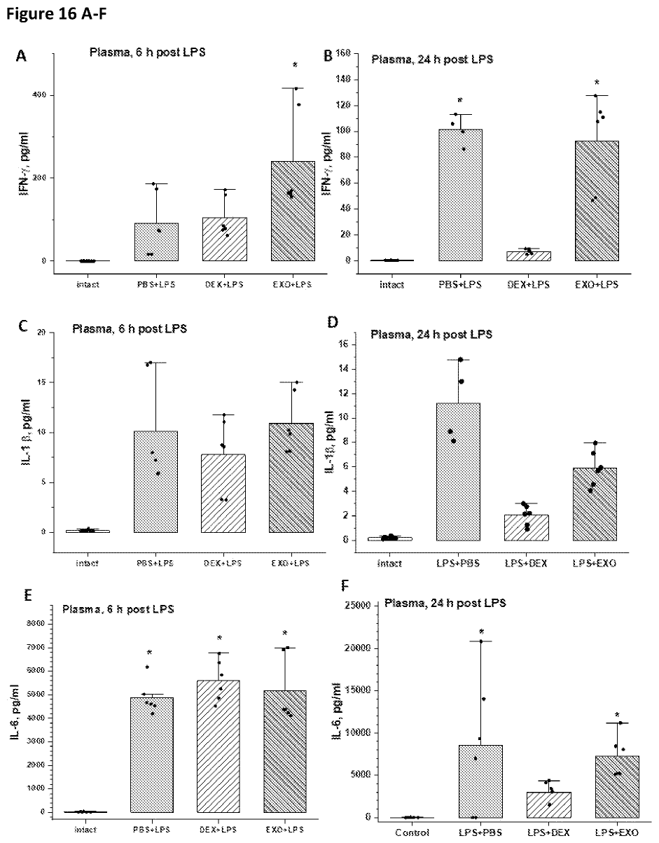

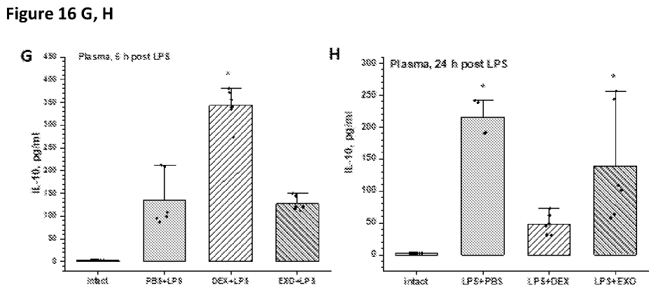

FIG. 16 shows cytokine measurements in plasma samples of the three LPS-induced groups with different treatments (PBS, Dex, Exo) compared to untreated control animals (intact). At 6 h post LPS induction, inflammatory cytokine levels were elevated in plasma samples of all groups compared to untreated controls. At 24 h post LPS induction, pro-inflammatory cytokines were reduced in both Dex- (positive control) and Exo-treated groups compared to the PBS treated group. For example, levels of both IFN-gamma and IL-1β rose in the PBS animals and declined in the Dex and Exo groups. Plasma IL6 rose in the Exo animals compared to the Dex group, but to a lesser degree than in the PBS treated animals, suggesting that Exo attenuated upregulation of IL6.

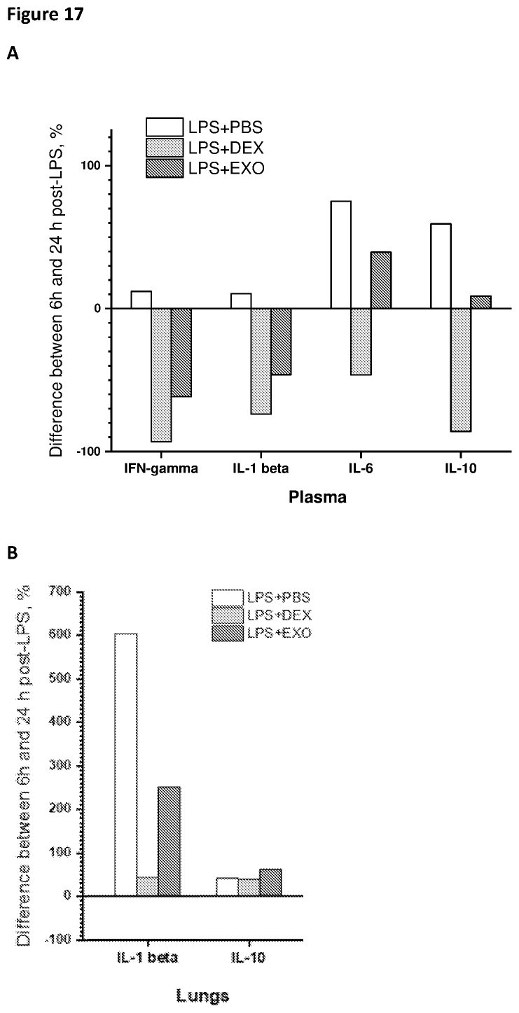

Interestingly from 6-24 h, IL10 was reduced in Dex treated mice. IL10 levels rose in Exo treated animals, but to a lesser degree than in PBS treated mice. Elevation of IL10 following LPS treatment alone is in line with the LPS control study where IL10 was upregulated between 6-24 h (data not shown). In this study, in Exo treated mice, IL10 was upregulated to a greater degree in lung samples (data not shown) than in plasma (but not to the degree seen in PBS animals). FIG. 17 shows a summary of the relative change of cytokine levels between 6 h and 24 h post LPS induction in plasma and lung samples.

Taken together, these experiments suggest that EVs exert a potent anti-inflammatory effect in the LPS model of sepsis.

TABLE 13
Scoring injury factors in pathology (each microscopic Scan
with ×400 magnification was divided into four parts)

| Definition | Score |
|---|---|
| There is no complication in any quarter. | 0 |
| In ¼ of them there is a complication. | 1 |
| In 2/4 of them there is a complication. | 2 |
| In ¾ of them there is a complication. | 3 |
| In 4/4 of them there is a complication. | 4 |

### Statistical Analysis

Experimental data were processed using the Statistica 10.0 software package. Data are shown in graphs as median (ranges). For intergroup comparison, the nonparametric Kruskal-Wallis's test was used, with adjustment for multiple comparisons The differences were considered statistically significant with a P-value equal to or less than 0.05.

### Example 16: Covid-19

While the factors that trigger the severe symptoms of some patients suffering from Covid-19 are not fully understood, all patients appear to share hyperinflammation characterized by, inter alia, increased inflammatory cytokines and chemokines in the blood. This project will investigate the “experimental treatments” for Covid-19 in BALB/c mice. The experimental treatments will be given to mice following the induction of a cytokine storm using the LPS model of inflammation [18].

Results of our preliminary study on treatment of treatment of mice with high Muse cell MSC exosomes shows that the pro-inflammatory cytokine IL-1β was reduced, while the anti-inflammatory marker IL-10 was slightly increased compared to controls in the lungs of exosome-treated mice in an LPS-induced sepsis model (FIG. 17). Hence, exosome treatment showed an effect on lung inflammation in a systemic inflammation model, which suggests therapeutic potential in the treatment of Covid-19.

### Experimental Plan:

100 male and female BALB/c mice, 8-12 week old, will be split into groups of 10. Groups will be assigned according to the permitted doses of the test preparations. Inflammation induced by adjuvants such as LPS (lipopolysaccharides), etc., will continue to be studied in all mice except the control group according to the established treatment plan.

Serum concentrations of inflammatory-specific cytokines and anti-inflammatory cytokines (such as: IL-6; TNF-α; IL-1 β; IL-10; IL-8/CXCL-8; IFN-α; IFN-β; IFN-γ; MCP1/CCL2; SDF-1/CXCL12 and/or others) will be measured Luminex analysis.

The effect of experimental treatments on changes in cytokine concentrations will be assessed at defined time points.

These results will help determine to what extent EVs can mitigate a cytokine storm, which invariably occurs in severe Covid-19 patients. The ability of the experimental treatments to suppress or relieve the overproduction of inflammatory cytokines suggests that they can reduce the damage inflicted by the SARS-CoV-2 virus as well as increase the speed of recovery, while reducing the long-term deleterious effects of the disease that can lead to Long Covid. Successful results will also support future experiments to test the restorative capacities of EVs in Long Covid patients.

### Example 17: Cartilage Regeneration

Cartilage is avascular in nature, which results in its inability to surmount an inflammatory response. Cartilage is therefore easily attacked by proinflammatory factors and oxidative stress, which if left untreated may progress to osteoarthritis. develop.

In order to test whether EVs of the invention can induce cartilage regeneration, EV100, EV500, and/or Muse cell derived secretomes will be tested in an animal model of osteoarthritis (monoiodoacetate (MIA) model).

Twenty-four healthy female New Zealand white rabbits 8 months of age will be used in the study. The animals will be divided into 4 treatment groups, each receiving a different dosing of MIA. The left knee joint capsule area will be injected by infra-patellar injection with 0.2 ml of an MIA (Sigma Aldrich) solution. The right knee will be injected with same volume of physiological serum solution as a control. EVs will be injected according to planned treatment dosage and schedule. Forty-five to 60 days after induction, the animals will be euthanized and the knees will be dissected. Joint cartilage of the two femoral condyles, the tibial plates and the menisci will be dissected to obtain a macroscopic score, following Laverty and colleagues, and as recommended by OARSI [19]. The severity of osteoarthritis, osteoarthritic synoviovite pathology will be assessed on the OARSI scale. The data for each group will be presented as a mean, and compared by ANOVA factor analysis of variance test to assess the intra-group reproducibility and the inter-group difference. The results from these experiments will further enable us to test high muse EV100 and EV500 treatment for the repair of articular cartilage in human joints.

### Example 18: Epigenetic Effect

Over recent years, the ability to estimate the biological age of individual tissues had been achieved through the analysis of DNA methylation based biomarkers. The combined study of high numbers of methylation marks throughout the genomes of animals and humans has resulted in the development of epigenetic clocks have become useful for determining aging in various tissues over time, and enabled us to further understand the biology of aging [20], [21]. In order to determine if Muse cell EVs can alter DNA methylation, i.e., the aging characteristics of cells that determine biological age, bone marrow and adipose MSCs will be obtained from old mice and cultured with the addition of perinatal, high Muse cell EVs and/or secretomes. Tools such as the Illumina 450K chip [22] and additional CpG methylation marker data will be used to analyze epigenetic age, before and after EV treatment to determine if the vesicles are able to reverse the biological age of the cells. For example, the methylation profiles and specific methylation CpG sites of the genes ITGA2B (cg25809905), ASPA (cg02228185), and PDE4C (cg17861230) have been compared in blood samples of patients with diseases associated with bone marrow failure. The results suggested accelerated biological age in these individuals [23]. As a functional readout of EV impact on the cells, a panel of markers of inflammation will be measured by Luminex analysis (ThermoFisher). EV-treated and untreated cells will also be administered to mice using the contact hypersensitivity model of inflammation to measure their anti-inflammatory effect [24].

If the EVs are able to reduce the biological age of the cells, then the possibility exists that the “younger” cells will be able to reduce inflammation. The epigenetically younger cells will likely provide a greater benefit to the inflamed animal model. The cells will also be administering to old mice to determine whether the biological age of the old animal tissues and organs becomes younger. These studies will help lead to a better understanding of the relationship between biological age and diseases of inflammation, and may lead to the ability to halt, slow, or even reverse the aging process.

The invention refers to the following further enumerated embodiments:
1. A plurality of extracellular vesicles (EVs) obtained from Muse cells or a tissue comprising Muse cells.
2. The plurality of EVs according to item 1, wherein the EVs have a mean diameter of about 1000 nm or less.
3. The plurality of EVs according to item 1 or 2, wherein the EVs have an mean diameter of less than 1000 nm, 900 nm, 800 nm, 700 nm, 600 nm, 500 nm, 400 nm, 300 nm or 200 nm.
4. The plurality of EVs according to any one of the preceding items, wherein substantially all EVs have a diameter of less than 550 nm.
5. The EVs according to any one of the preceding items, wherein the EVs are selected from a group comprising exosomes (EV100) and/or microvesicles (EV500), wherein the plurality of EVs have an average diameter between 100 nm to about 550 nm.
6. The plurality of EVs according to any one of the preceding items, wherein the D90 value of said plurality of EVs is 500 nm or less than 500 nm.
7. The plurality of EVs according to any one of the preceding items, wherein the D90 value is about 320-420 nm.
8. The plurality of EVs according to any one of the preceding items, wherein the D90 value is about 330 nm.
9. The plurality of EVs according to item 7 or 8, wherein the D10 value is about 200 nm.
10. The plurality of EVs according to any one of the preceding items, wherein substantially all EVs have a diameter of 550 nm or less.
11. The plurality of EVs according to any one of items 5 to 9, wherein the microvesicles (EV500) have a mean diameter of about >220 to 550 nm, 230 to 500 nm, or 300 to 450 nm.
12. The plurality of EVs according to any one of items 5 to 10, wherein the microvesicles (EV500) have a diameter of between 220 to about 550 nm, preferably between 250 nm to 400 nm.
13. The plurality of EVs according to any one of the preceding items, wherein at least 20%, 30%, 40%, 50%, 60%, 70%, 80%, or 90% of the EVs are microvesicles (EV500).
14. The plurality of EVs according to any one of the preceding items, wherein substantially all EVs are microvesicles (EV500).
15. The plurality of EVs according to any one of items 1-6, wherein the D90 value is about 150-200 nm.
16. The plurality of EVs according to any one of items 1-6, wherein the D90 value is about 165 nm.
17. The plurality of EVs according to item 15 or 16, wherein the D10 value is about 40-100 nm.
18. The plurality of EVs according to item 17, wherein the D10 value is about 45 nm.
19. The plurality of EVs according to any one of items 15 to 18, wherein the EVs comprise exosomes (EV100) and have an average diameter of about 100 nm to 150 nm.
20. The plurality of EVs according to any one of item 15 to 19, wherein substantially all EVs have a diameter of 220 nm or less.
21. The plurality of EVs according to any one of items 1-5 or item 20, wherein substantially all EVs have a diameter of about 30 nm to 220 nm.
22. The plurality of EVs according to item 21, wherein the exosomes have a mean diameter of about 30 to 200 nm, 50 to 170 nm, or about 90 to 140 nm.
23. The plurality of EVs according to any one of items 1 to 5, wherein at least 20%, 30%, 40%, 50%, 60%, 70%, 80%, or 90% of the EVs are exosomes (EV100).
24. The plurality of EVs according to any one of items 1 to 6, wherein substantially all EVs are exosomes (EV100).
25. The plurality of EVs according to any one of items 1 to 5, or 15 to 24, wherein the exosomes (EV100) express at least one of CD9, CD63, CD81 or tetraspanins.
26. The plurality of EVs according to any one of the preceding items, wherein the Muse cells are obtained from a tissue selected from a group comprising mesenchymal stem cells, adipose-derived stem cells, bone-marrow-derived stem cells, placenta, umbilical cord, cord blood, Wharton's jelly, cartilage, adipose tissue, bone marrow or dermal fibroblastic tissue.
27. The plurality of EVs according to item 26, wherein the Muse cells are obtained from placenta, umbilical cord, cord blood and/or Wharton's jelly.
28. The plurality of EVs according to item 26, wherein the Muse cells are obtained from placenta.
29. The plurality of EVs according to item 26, wherein the Muse cells are obtained from umbilical cord.
30. The plurality of EVs according to item 26, wherein the Muse cells are obtained from cord blood.
31. The plurality of EVs according to item 26, wherein the Muse cells are obtained from Wharton's jelly.
32. The plurality of EVs according to any one of the preceding items, wherein the Muse cells are obtained from a human subject.
33. The plurality of EVs according to any one of the preceding items, wherein the Muse cells are obtained from a non-human subject.
34. The plurality of EVs according to item 32 or 33, wherein the Muse cells are obtained from an adult subject or a subject, which is not an adult.
35. The plurality of EVs according to any one of the preceding items, wherein the Muse cells are undifferentiated Muse cells.
36. The plurality of EVs according to any one of the preceding items, wherein the Muse cells express SSEA-3.
37. The plurality of EVs according to item 36, wherein the Muse cells express CD105.
38. The plurality of EVs according to item 35, wherein the Muse cells express SSEA-3 and CD105.
39. The plurality of EVs according to any one of item 36-38, wherein the Muse cells further express CD90, CD73, or both.
40. The plurality of EVs according to item 39, wherein the Muse cells express SSEA-3, CD105 and CD90.
41. The plurality of EVs according to item 39, wherein the Muse cells express SSEA-3, CD105 and CD73.
42. The plurality of EVs according to any one of the preceding items, wherein the Muse cells do not express CD45.
43. The plurality of EVs according to any one of the preceding items, wherein the Muse cells express SSEA-3 and do not express CD45.
44. The plurality of EVs according to any one of the preceding items, wherein the EVs are purified and substantially free of any contaminants.
45. The plurality of EVs according to item 44, wherein the contaminant comprises one or more of endotoxins, mycoplasma, pathogens or pathobionts.
46. The plurality of EVs according to any one of the preceding items, wherein the EVs are purified by ultrafiltration or sterile filtration.
47. A composition comprising a plurality of EVs according to any one of the preceding items.
48. The composition according to item 47, further comprising EVs from mesenchymal stem cells (MSCs).
49. The composition according to item 47 or 48, wherein the quantity of EVs from Muse cells is greater than the quantity of EVs derived from MSCs.
50. The composition of any one of items 47 to 49, further comprising a biocompatible matrix, wherein the EVs are optionally attached to said biocompatible matrix.
51. The composition according to item 50, wherein the biocompatible matrix comprises collagen, thrombin, gelatin, alginate, or another naturally-occurring basement membrane product.
52. The composition according to any one of items 47 to 51 comprising EVs at a concentration of at least 1*10×E5 EVs/ml.
53. The composition according to any one of items 47 to 51 comprising EVs at a concentration of at least 1*10×E6 EVs/ml.
54. The composition according to any one of items 47 to 51 comprising EVs at a concentration of at least 1*10×E7 EVs/ml.
55. The composition according to any one of items 47 to 51 comprising EVs at a concentration of at least 1*10×E8 EVs/ml.
56. The composition according to any one of items 47 to 51 comprising EVs at a concentration of at least 1*10×E9 EVs/ml.
57. The composition according to any one of items 47 to 51 comprising EVs at a concentration of at least 1*10×E10 EVs/ml.
58. The composition according to any one of items 47 to 57, wherein the composition is lyophilized.
59. The composition of item 58, wherein the EVs comprised therein are stable for at least 6 months, 12 months, 18 months, 24 months or 36 months at ambient temperature.
60. The composition of item 58 or 59, wherein the EVs are present at a concentration of at least 1*10×E6, 1*10×E7, 1*10×E8, 1*10×E9, 1*10×E10, or 1*10×E11 EVs/gram.
61. A method for preparing EVs from Muse cells comprising the steps of:
   (a) providing Muse cells;
   (b) culturing the Muse cells of (a) in a first medium for a time period sufficient to expand Muse cells in an undifferentiated state;
   (c) culturing the Muse cells of (c) in a second medium for a time period sufficient to induce production of EVs, wherein the EVs are released into the second medium; and
   (d) harvesting the EVs for one or more times, wherein each harvesting comprises collecting the second medium comprising the EVs to obtain a harvest.
62. The method of item 61, wherein the Muse cells of (a) are provided as a single cell suspension.
63. The method of item 61 or 62, wherein the culturing of Muse cells of (b) comprises expanding the Muse cells by a factor of at least 2, 5, 10, 20, 30, 40, 50 or 60.
64. The method of any one of items 61 to 63, wherein the undifferentiated Muse cells of (b) express SSEA-3.
65. The method of any one of items 61 to 64, wherein each harvesting of step (d) comprises replacing the medium comprising the EVs with a fresh second medium.
66. The method of any one of items 61 to 65, wherein the harvesting (d) is repeated once, twice, three times or more.
67. The method of any one of items 61 to 66, wherein after harvesting (d) the Muse cells are re-plated at sub-confluent density, and repeating steps (b)-(d).
68. The method of any one of items 61 to 67, wherein between steps (b) and (c) the cells are re-plated at lower density (i.e. passaged) to further expand the Muse cell quantity.
69. The method of any one of items 61 to 68, wherein between steps (b) and (c) the cells are passaged.
70. The method of any one of items 61 to 68, wherein the Muse cells are cultured in pre-coated culture dishes.
71. The method according to item 70, wherein the pre-coated culture dish comprises coating with said dish with poly-D-Lysine, Matrigel, or collagen prior to plating the Muse cells.
72. The method according to any one of items 61 to 71, further comprising a step
   (e) processing of the harvest obtained in (d).
73. The method according to item 72, further comprising a step
   (f) subjecting the processed harvest of step (e) to one or more purification steps, wherein the purification steps optionally comprise ultrafiltration.
74. The method according to any one of items 61 to 73, wherein the Muse cells of (a) are obtained from mesenchymal stem cells, adipose-derived stem cells, bone-marrow-derived stem cells, placenta, umbilical cord, cord blood, Wharton's jelly, cartilage, adipose tissue, bone marrow or dermal fibroblastic tissue.
75. The method according to item 74, wherein the Muse cells are obtained from a human subject.
76. The method according to item 74 or 75, wherein the Muse cells are obtained from an adult subject or a subject, which is not an adult subject.
77. The method according to any one of item 74 to 76, wherein the Muse cells are obtained from umbilical cord, cord blood, placenta or Wharton's jelly.
78. The method according to item 77, wherein the Muse cells are obtained from umbilical cord.
79. The method according to item 77, wherein the Muse cells are obtained from cord blood.
80. The method according to item 77, wherein the Muse cells are obtained from placenta.
81. The method according to item 77, wherein the Muse cells are obtained from Wharton's jelly.
82. The method according to any one of items 61 to 81, wherein
   i. the first medium and second medium are not identical;
   ii. the first medium comprises animal or human proteins, such as animal or human cord blood plasma, and/or PRP lysate;
   iii. the second medium does not comprise animal or human proteins; and/or
   iv. the second medium comprises cytokines, biologically active proteins, ITS, FGF, VEGF, IGF-1 and/or glucose;
   v. between step (a) and (b), the cells are seeded in a plating medium, which is different to the first medium and second medium;
wherein two, three, four or five of (i) to (v) may be independently combined with each other.
83. The method according to any one of items 61 to 82, wherein the first medium induces preferential proliferation of Muse cells over MSCs, thereby resulting in an enrichment of Muse cells.
84. The method according to any one of items 61 to 83, wherein Muse cells after step (b) are enriched at least by a factor of 2, 3, 4, 5, 10, 100, 10³, 10⁴, 10⁵ or more relative to the amount of Muse cells present in step (a).
85. The method according to any one of items 61 to 84, wherein the undifferentiated state of the Muse cell of (b) is characterized by expression of SSEA-3.
86. The method according to item 85, wherein the undifferentiated Muse cell further expresses at least one or two additional markers selected from the group consisting of CD90, CD73, CD105, NANOG, Oct-4, Sox-2, Klf4 and c-Myc.
87. The method according to any one of items 61 to 86, wherein Muse cells are expanded at least 10, 100, 10³, 10⁴, 10⁵-fold relative to the concentration of Muse cells in the tissue the Muse cells were obtained from.
88. The method according to any one of items 61 to 87, wherein the culture of step (c) comprises at least 10% Muse cells.
89. The method according to any one of items 61 to 87, wherein the culture of step (c) comprises at least 15% Muse cells.
90. The method according to any one of items 61 to 87, wherein the culture of step (c) comprises at least 20% Muse cells.
91. The method according to any one of items 61 to 87, wherein the culture of step (c) comprises at least 25% Muse cells.
92. The method according to any one of items 61 to 87, wherein the culture of step (c) comprises at least 30% Muse cells.
93. The method according to any one of items 61 to 87, wherein the culture of step (c) comprises at least 35% Muse cells.
94. The method according to any one of items 61 to 87, wherein the culture of step (c) comprises at least 40% Muse cells.
95. The method according to any one of items 61 to 87, wherein the culture of step (c) comprises at least 45% Muse cells.
96. The method according to any one of items 61 to 87, wherein the culture of step (c) comprises at least 50% Muse cells.

97. The method according to any one of items 61 to 87, wherein the culture of step (c) comprises at least 60% Muse cells.
98. The method according to any one of items 61 to 87, wherein the culture of step (c) comprises at least 70% Muse cells.
99. The method according to any one of items 61 to 87, wherein the culture of step (c) comprises at least 80% Muse cells.
100. The method according to any one of items 61 to 99, wherein the harvest of step (d) or (e) comprises at least 105, 10⁶, 10⁷, 10⁸, or more EVs/ml.
101. The method according to any one of items 61 to 100, wherein the processing (e) further comprising a step of centrifuging to remove cellular debris.
102. The method of any one of items 61 to 101, wherein the purification step (f) comprises one or more of size exclusion chromatography, membrane filtration, normal flow filtration, tangent flow filtration, ultrafiltration, polymer precipitation, immunological separation, magnetic immunocapture, ultracentrifugation, density gradient centrifugation, or size exclusion chromatography.
103. The method of item 102, wherein the purification step (f) comprises tangential flow filtration.
104. The method of item 102, wherein the purification step (f) comprises ultrafiltration.
105. The method of item 102, wherein the purification step (f) comprises magnetic immunocapture.
106. The method of item 103 or 104, wherein the tangential flow filtration or ultrafiltration comprises a first filter having a first pore size, and optionally a second filter having a second pore size.
107. The method of item 106, wherein the first filter has a first pore size sufficient for isolating a plurality of substantially pure EV500 of item 4-14.
108. The method of any one of items 106 or 107, wherein the second filter has a pore size sufficient for isolating a plurality of substantially pure EV100 of items 15 to 25.
109. The method of any one of items 61 to 108, further comprising a step of
   (g) concentrating the obtained EVs.
110. The method of item 109, wherein the EVs are present in a concentration of at least 10⁸, 10⁹, 10¹⁰, 10¹¹, 10¹², 10¹³, or more EVs/ml.
111. The method of any one of items 61 to 109, further comprising a step of lyophilizing the EVs to obtain lyophilized EVs.
112. The method of item 111, wherein the lyophilized EVs comprise at least 10⁸ EVs, at least 107 EVs, at least 108 EVs, or at least 10° EVs.
113. The method of any one of items 61 to 112, wherein the expression of at least one of SSEA-3, CD105, CD90, CD45, NANOG, Oct-4, Sox-2, Klf4 or c-Myc in or on Muse cells is determined prior to or during step (b) or (c).
114. The method of item 113, wherein the expression of SSEA-3, CD105, CD90 or CD45 is determined by cytometry, preferably FACS or MACS.
115. A plurality of EVs obtainable by the method of any one of items 61 to 114.
116. A pharmaceutical composition comprising
   (i) a pharmaceutically effective amount of a plurality of EVs of any one of items 1 to 46;
   (ii) a pharmaceutically effective amount of the composition according to any one of items 47 to 60; or
   (iii) a pharmaceutically effective amount of the EVs according to item 115, and a pharmaceutically acceptable carrier.
117. The pharmaceutical composition according to item 116, further comprising an immunomodulatory agent, anti-inflammatory agent or therapeutic agent.
118. The pharmaceutical composition according to item 117, wherein the immunomodulatory agent is a cytokine, miRNA or IncRNA.
119. The pharmaceutical composition according to any one of items 116 to 118, wherein said composition comprises an emulsifying agent, an excipient, a pH buffering agent, and/or an adjuvant, wherein said adjuvant enhances the efficacy of the pharmaceutical composition.
120. The pharmaceutical composition according to item 119, wherein the excipient is selected from the group consisting of inert fillers or diluents, such as sucrose, sorbitol, sugar, mannitol, micro crystalline cellulose, starches, including potato starch, calcium carbonate, sodium chloride, lactose, calcium phosphate, calcium sulfate or sodium phosphate; crumbling agents and disintegrants, for example cellulose derivatives, including microcrystalline cellulose, starches, including potato starch, sodium croscarmellose, alginates or alginic acid and chitosans; binding agents, for example sucrose, glucose, sorbitol, acacia, alginic acid, sodium alginate, gelatin, starch, pregelatinized starch, microcrystalline cellulose, aluminum magnesium silicate, sodium carboxymethylcellulose, methylcellulose, hydroxypropyl methylcellulose, ethylcellulose, polyvinylpyrrolidone, polyvinyl acetate or polyethylene glycol, and chitosans; lubricating agents, including glidants and antiadhesive agents, for example magnesium stearate, zinc stearate, stearic acid, silicas, hydrogenated vegetable oils or talc.
121. The pharmaceutical compositions according to any one of items 116 to 120, wherein said composition is formulated for administration via the rectal, intranasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir into the subject.
122. The pharmaceutical compositions according to item 121, wherein the composition is formulated for administration intranasally or intravenously.
123. The pharmaceutical composition according to any one of items 116 to 122 for use in therapy.
124. The pharmaceutical composition of any one of items 116 to 122 for use in a method of treating a cancer or tumor in a subject, said method comprising administering a therapeutically effective amount of the pharmaceutical composition to the subject.
125. The pharmaceutical composition for use of item 124, wherein said method slows down tumor progression, reduces the size of the tumor and/or ameliorates adverse effects associated with the presence of the tumor or cancer.
126. The pharmaceutical composition for use of item 124 or 125, said method comprising administering to the subject therapeutically effective amount of the composition, wherein said composition enhances tumor targeting of the subject's immune system.
127. The pharmaceutical composition for use of any one of items 124 to 126, wherein the tumor burden in the subject is reduced by said composition.
128. The pharmaceutical composition for use of any one of items 124 to 127, wherein the release of exosomes or microvesicles from the tumor cells are reduced by said composition.
129. The pharmaceutical composition for use according to any one of items 124 to 128, wherein the cancer is a cancer of the breast, liver, pancreas, lymph nodes, lung, skin, bone marrow, adipose tissue, nerve tissue, glioma, stomach, colon, blood, kidney, or endometrium. cancer of the breast, liver, pancreas, lymph nodes, lung, skin, bone marrow, adipose tissue, nerve tissue, glioma, stomach, colon, blood, kidney, or endometrium.
130. The pharmaceutical composition for use according to item 129, wherein the cancer is breast cancer, optionally wherein the breast cancer is recurrent breast cancer.
131. The pharmaceutical composition for use according to item 129, wherein the cancer is lung carcinoma.
132. The pharmaceutical composition for use according to item 129, wherein the cancer is head and neck squamous cell carcinoma.
133. The pharmaceutical composition of any one of items 116 to 122 for use in a method of treating a growth, vascularization, or metastasis of a tumor in a subject.
134. The pharmaceutical composition for use according to any one of items 124 to 133, said method comprising administration of at least one additional anti-cancer agent.
135. The pharmaceutical composition for use according to item 134, wherein said anti-cancer agent is an alkylating agent, antimetabolite, hormone, miRNA, chemotherapeutic agent, or cytotoxic agent.
136. The pharmaceutical composition for use according to item 134, wherein said anti-cancer agent is a therapeutic miRNA, therapeutic IncRNA protein, chemotherapeutic agent, anti-mitotic agent, therapeutic antibody or fragment thereof.
137. The pharmaceutical composition for use according to any one items 134 to 136, wherein said anti-cancer agent is conjugated to or encapsulated in the EVs comprised in said pharmaceutical composition.
138. The pharmaceutical composition for use according to item 137, wherein conjugation or encapsulation of the anti-cancer agent to the EVs does not inhibit the therapeutic efficacy of the EVs.
139. The pharmaceutical composition according to any one of items 116 to 122 for use in a method of treating inflammation or an inflammatory disorder in a subject in need thereof, said method comprising administering a therapeutically effective amount of said composition to said subject.
140. The pharmaceutical composition for use of item 139, wherein the inflammation or inflammatory disorder is a chronic inflammation or inflammatory disorder.
141. The pharmaceutical composition for use of item 139, wherein the inflammation or inflammatory disorder is an acute inflammation or inflammatory disorder.
142. The pharmaceutical composition for use of any one of items 139 to 141, wherein the inflammation or inflammatory disorder is a recurrent inflammation or inflammatory disorder.
143. The pharmaceutical composition for use according to any one of items 139 to 142, wherein the inflammatory disease comprises rheumathoid arthritis (RA), Osteoarthritis, Type 2 diabetes mellitus, Type 1 diabetes mellitus, inflammatory bowel disease, asthma, endometriosis, cancer, alopecia, inflammatory arthritis, psoriatic arthritis, lupus, juvenile idiopathic arthritis, gout, systemic lupus erythematosus, and seronegative spondyloarthropathies, relapsing polychondritis neurological inflammation, multiple sclerosis, migraine, Traumatic Brain Injury (TBI) and Chronic Traumatic Encephalopathy (CTE), Covid-19, long Covid-19, Celiac Disease, Inflammatory Bowel Disease (IBD), encephalitis, chronic obstructive pulmonary disease (COPD), inflammatory osteolysis, Crohn's disease, ulcerative colitis, allergic disorders, septic shock, pulmonary fibrosis (e.g., idiopathic pulmonary fibrosis), inflammatory vacultides (e.g., polyarteritis nodosa, Wegner's granulomatosis, Takayasu's arteritis, temporal arteritis, and lymphomatoid granulomatosus), post-traumatic vascular angioplasty (e.g. restenosis after angioplasty), undifferentiated spondyloarthropathy, undifferentiated arthropathy, arthritis, inflammatory osteolysis, chronic hepatitis, chronic inflammation resulting from chronic viral or bacterial infections, and acute inflammation, such as sepsis.
144. The pharmaceutical composition for use according to any one of items 139 to 143, said method comprising administration of at least one additional anti-inflammatory agent.
145. The pharmaceutical composition for use according to item 144, wherein said anti-inflammatory agent comprises an anti-inflammatory peptide or protein, or an anti-inflammatory nucleic acid.
146. The pharmaceutical composition for use according to item 145, wherein said anti-inflammatory nucleic acid comprises DNA, RNA.
147. The pharmaceutical composition for use according to item 146, wherein said anti-inflammatory RNA comprises noncoding RNAs (ncRNAs), or microRNAs (miRNAs).
148. The pharmaceutical composition for use according to item 147, wherein said miRNAs comprise one or more of miR-199a-3p, miR-143-3p, miR-21-5p, miR-125b-5p, let-7b-5p, miR-29a-3p, let-7i-5p, miR-125a-5p, miR-16-5p, miR-221-3p, miR-432-5p, miR-127-3p, miR-382-5p, miR-146a-5p, miR-431-5p, let-7a-5p, miR-199b-3p, miR-100-5p, miR-6504-3p, miR-26a-5p, let-7a-5p, let-7f-5p, miR-26a-5p, miR-431-5p, miR-126-3p, miR-181a-5p, let-7e-5p.
149. The pharmaceutical composition for use according to item 148, wherein said miRNAs comprise miR-21-5p and/or let-7b-5p.
150. The pharmaceutical composition for use according to any one of items 147 to 149, wherein said miRNAs are negative regulators of inflammation.
151. The pharmaceutical composition for use according to any one of items 139 to 150, wherein the subject suffers from Covid-19 associated inflammation.
152. The pharmaceutical composition for use according to item 151, wherein the inflammation is a neuroinflammation of the brain and/or choroid plexus.
153. The pharmaceutical composition for use according to item 151, wherein the subject suffers from long Covid-19 associated inflammation.
154. The pharmaceutical composition for use according to any one of items 139 to 150, wherein the subject suffers from pneumonitis, i.e. an inflammation of the lung.
155. The pharmaceutical composition for use according to any one of items 139 to 150, wherein the subject suffers from hepatitis, i.e. an inflammation of the liver.
156. The pharmaceutical composition for use according to any one of items 139 to 150, wherein the subject suffers from nephritis, i.e. an inflammation of the kidney.
157. The pharmaceutical composition of any one of items 139 to 150 for use in a method of restoring health in a subject in need thereof, said method comprising administering a therapeutically effective amount of said composition to said subject.
158. The pharmaceutical composition of any one of items 139 to 150 for use in a method of regeneration in a subject in need thereof, said method comprising administering a therapeutically effective amount of said composition to said subject.
159. The pharmaceutical composition for use of item 158, wherein the subject suffers from stroke, myocardial infarction, age-related cognitive decline, or age-related loss of motor function.
160. The pharmaceutical composition for use according to any one of items 157 to 159, wherein the subject is above the age of 50, 60, 70 or 80 years.
161. The pharmaceutical composition for use according to any one of items 157 to 160, wherein the composition effects an increase of one or more telomeres of a cell sample of the subject.
162. The pharmaceutical composition for use according to any one of items 157 to 161, wherein the regeneration comprises reversing the status of one or more epigenetic marks to a status correlated with a subject of younger age.
163. The pharmaceutical composition for use according to item 162, wherein the one or more epigenetic marks are DNA methylation marks.
164. The pharmaceutical composition according to any one of items 116 to 122 for use in a method of treating cartilage degeneration in subject in need thereof, said method comprising administering a therapeutically effective amount of said composition to said subject.
165. The pharmaceutical composition for use according to item 164, wherein the subject suffers from osteoarthritis.
166. The pharmaceutical composition for use according to any one of items 116 to 122 for use in a method of treating Covid-19 or long Covid-19 in a subject in need thereof, wherein method comprises administration of said pharmaceutical composition to the subject.
167. The pharmaceutical composition according to any one of items 116 to 122 for use in preventing or treating senescence of a cell of the subject.
168. The pharmaceutical composition according to any one of items 116 to 122 for use in a method of promoting wound healing in a subject.
169. The pharmaceutical composition according to any one of items 116 to 122 for use in a method of increasing vascularization of a wound bed in a subject in need thereof.
170. The pharmaceutical composition according to any one of items 116 to 122 for use in a method of increasing epithelization of a wound bed in a subject in need thereof.
171. The pharmaceutical composition for use according to any one of items 167 to 170, said method comprising administering a therapeutically effective amount of said composition to the subject.
172. The pharmaceutical composition for use of item 169 or 170, wherein the wound is healed in less time than the wound would heal without being treated with said composition.
173. The pharmaceutical composition for use according to any one of items 123 to 172, wherein the subject is a mammal, preferably, wherein the mammal is a human, a canine, a feline, a porcine or an equine.
174. The pharmaceutical composition for use of item 173, wherein the subject is a human.
175. The pharmaceutical composition for use according to any one of items 123 to 172, wherein the pharmaceutical composition is administered locally or systemically to the subject.
176. The pharmaceutical composition for use according to item 175, wherein the method comprises administering the pharmaceutical composition systemically in an amount ranging from 10³-10⁶ EVs per kilogram of total body weight.
177. The pharmaceutical composition for use according to item 175 or 176, wherein the route of administration is selected from intravenous, intramuscular, subcutaneous, rectal, vaginal, inhalation, oral, buccal, sublingual, transdermal, ocular, intrathecal or nasal administration.
178. The pharmaceutical composition for use according to item 175, wherein the local administration comprises providing the pharmaceutical composition in an amount ranging from 10³-10¹¹ EVs into a localized tissue or anatomical space.
179. Non-therapeutic use of the pharmaceutical composition of any one of items 116 to 122 for preventing hair loss or promoting hair growth in a subject.
180. The non-therapeutic use of item 179, wherein the subject is a mammal, optionally wherein the subject is a human.
181. The non-therapeutic use according to item 179 or 180, comprising topical administration of the pharmaceutical composition to the afflicted area in need of preventing hair loss or promoting hair growth in the subject.
182. The non-therapeutic use according to any one of items 179 to 181, wherein the topical administration comprises administration of at least 10³, 10⁴, 10⁵, 10⁶, 10⁷, 10⁸, 10⁹ EVs to the subject.
183. Non-therapeutic use of the pharmaceutical composition of any one of items 116 to 122 for improving the appearance of wrinkles of the skin of a subject.
184. The non-therapeutic use according to item 183, wherein the subject is human.
185. The non-therapeutic use according to item 183 or 184, wherein the abundance and/or depth of the wrinkles is reduced.
186. The non-therapeutic use according to any one of items 183 to 185, comprising topical administration of the pharmaceutical composition to the afflicted area in need of improving the appearance of wrinkles of the skin of the subject.
187. The non-therapeutic use according to item 186, wherein the topical administration comprises administration of at least 10³, 10⁴, 10⁵, 10⁶, 10⁷, 10⁸, 10⁹ 10¹⁰, 10¹¹ EVs to the subject.
188. The non-therapeutic use according to item 187, wherein the topical administration is by cream, gel, ointment, foam, aerosol or patch.
189. A dosage form comprising the pharmaceutical composition according to any one of items 116 to 122.
190. The dosage form according to item 189, wherein said dosage form is suitable for administration by injection, infusion, drops, patch, cream or ointment.
191. The dosage form according to 189 or 190, wherein said dosage form is selected from the group comprising tablet, capsule, gel-capsule, aerosol, liquid solution, eye-drop, film, cream, gel, film, or ointment.
192. Use of the pharmaceutical composition according to any one of items 116 to 122 for the manufacture of a medicament.
193. A stable lyophilized formulation comprising the plurality of EVs of any one of items 1-25 or the pharmaceutical composition of any one of items 116 to 122.
194. The stable lyophilized formulation of item 193, wherein said formulation is suitable for administering to a subject, wherein the subject is optionally an animal.
195. The stable lyophilized formulation according to item 193 or 194, said formulation comprising at least 106 EVs, at least 10⁷ EVs, at least 10⁸ EVs, or at least 10⁹ EVs.
196. The stable lyophilized formulation according to any one of items 193 to 195, wherein the lyophilized EVs maintain at least 60% of the functional efficacy after resuspension relative to the EVs prior to lyophilization.
197. The pharmaceutical composition for use according to any one of items 123 to 178, wherein the pharmaceutical composition is administered systemically.
198. The pharmaceutical composition for use according to item 197, wherein the pharmaceutical composition is administered by intravenous injection.
199. The pharmaceutical composition for use according use according to any one of items 123 to 178, wherein the pharmaceutical composition is administered locally.
200. The pharmaceutical composition for use according to item to any one of items 123 to 178, wherein the pharmaceutical composition is administered via the rectal, nasal, buccal, vaginal, subcutaneous, intracutaneous, intravenous, intraperitoneal, intramuscular, intraarticular, intrasynovial, intrasternal, intrathecal, intralesional, or intracranial route, or via an implanted reservoir.
201. The pharmaceutical composition for use according to item to any one of items 123 to 178, wherein the pharmaceutical composition is administered by topical application, optionally, wherein said topical application comprises administration of eye-drops.
202. The pharmaceutical composition for use according to items 139 to 149, wherein the inflammatory disease is rheumatoid arthritis.
203. The pharmaceutical composition for use according to items 139 to 149, wherein the inflammatory disease is psoriasis.
204. The pharmaceutical composition for use according to items 139 to 149, wherein the inflammatory disease is migraine.
205. The pharmaceutical composition for use according to items 139 to 149, wherein the inflammatory disease is Covid-19.
206. The pharmaceutical composition for use according to items 139 to 149, wherein the inflammatory disease is rosacea.
207. The pharmaceutical composition for use according to items 139 to 149, wherein the inflammatory disease is melasma.
208. The pharmaceutical composition for use according to items 139 to 149, wherein the inflammatory disease is diabetic neuropathy.
209. The method according to items 61 to 66, wherein steps (b) and (c) of the method are performed in a bioreactor.
210. The method according to item 209, wherein the culture is performed at large scale.
211. The method according to item 210, wherein the large scale culture comprises a total volume of at least 1 L of medium.
212. The method according to items 61 to 66, wherein Muse cells are cultured on microcarriers.
213. The method according to item 212, wherein the microcarriers are coated with L-Lysine or collagen.

### REFERENCES

[1]P. P. Bruno, F. Carpino, G. Carpino, and A. Zicari, “An overview on immune system and migraine,” Eur Rev Med Pharmacol Sci, vol. 11, no. 4, pp. 245-248, 2007.
[2]I. B. McInnes, C. D. Buckley, and J. D. Isaacs, “Cytokines in rheumatoid arthritis—shaping the immunological landscape,” Nat Rev Rheumatol, vol. 12, no. 1, Art. no. 1, Jan. 2016, doi: 10.1038/nrrheum.2015.171.
[3]J. E. Gudjonsson, A. Johnston, and C. N. Ellis, “Novel systemic drugs under investigation for the treatment of psoriasis,” Journal of the American Academy of Dermatology, vol. 67, no. 1, pp. 139-147, Jul. 2012, doi: 10.1016/j.jaad.2011.06.037.
[4]A. Hasanvand, “COVID-19 and the role of cytokines in this disease,” Inflammopharmacol, vol. 30, no. 3, pp. 789-798, Jun. 2022, doi: 10.1007/s10787-022-00992-2.
[5]S. Montazersaheb et al., “COVID-19 infection: an overview on cytokine storm and related interventions,” Virol J, vol. 19, no. 1, p. 92, May 2022, doi: 10.1186/s12985-022-01814-1.
[6]D. Rodrigues-Braz, M. Zhao, N. Yesilirmak, S. Aractingi, F. Behar-Cohen, and J.-L. Bourges, “Cutaneous and ocular rosacea: Common and specific physiopathogenic mechanisms and study models,” Mol Vis, vol. 27, pp. 323-353, May 2021.
[7]R. Sarkar, A. Bansal, and P. Ailawadi, “Future therapies in melasma: What lies ahead?,” IJDVL, vol. 86, p. 8, Jan. 2020, doi: 10.4103/ijdvl.IJDVL_633_18.
[8]H. Zheng et al., “Proinflammatory cytokines predict the incidence of diabetic peripheral neuropathy over 5 years in Chinese type 2 diabetes patients: A prospective cohort study,” eClinicalMedicine, vol. 31, Jan. 2021, doi: 10.1016/j.eclinm.2020.100649.
[9]Y. I. Lee, S. Choi, W. S. Roh, J. H. Lee, and T.-G. Kim, “Cellular Senescence and Inflammaging in the Skin Microenvironment,” Int J Mol Sci, vol. 22, no. 8, p. 3849, Apr. 2021, doi: 10.3390/ijms22083849.
[10]K. Baghaei et al., “Isolation, differentiation, and characterization of mesenchymal stem cells from human bone marrow,” Gastroenterol Hepatol Bed Bench, vol. 10, no. 3. pp. 208-213, 2017.
[11]E. Lázaro-Ibáñez et al., “Different gDNA content in the subpopulations of prostate cancer extracellular vesicles: Apoptotic bodies, microvesicles, and exosomes,” The Prostate, vol. 74, no. 14, pp. 1379-1390, 2014, doi: 10.1002/pros.22853.
[12]J. wei Li, L. Wei, Z. Han, and Z. Chen, “Mesenchymal stromal cells-derived exosomes alleviate ischemia/reperfusion injury in mouse lung by transporting anti-apoptotic miR-21-5p,” European Journal of Pharmacology, vol. 852, pp. 68-76, Jun. 2019, doi: 10.1016/j.ejphar.2019.01.022.
[13]D. Ti et al., “LPS-preconditioned mesenchymal stromal cells modify macrophage polarization for resolution of chronic inflammation via exosome-shuttled let-7b,” Journal of Translational Medicine, vol. 13, no. 1, pp. 1-14, 2015, doi: 10.1186/s12967-015-0642-6.
[14]J. Kannaiyan, P. Shukla, and F. Mahuvawalla, “Acute Toxicity Evaluation and Safety Study of Allogeneic Wharton's Jelly-Derived Mesenchymal Stromal Cells Therapy in the Animal Model,” Rehabilitation Science, vol. 7, no. 2, Art. no. 2, May 2022, doi: 10.11648/j.rs.20220702.12.
[15]D. H. Ha et al., “Toxicological evaluation of exosomes derived from human adipose tissue-derived mesenchymal stem/stromal cells,” Regulatory Toxicology and Pharmacology, vol. 115, p. 104686, Aug. 2020, doi: 10.1016/j.yrtph.2020.104686.
[16]C. J. Morris, “Carrageenan-Induced Paw Edema in the Rat and Mouse,” in Inflammation Protocols, P. G. Winyard and D. A. Willoughby, Eds. Totowa, NJ: Humana Press, 2003, pp. 115-121. doi: 10.1385/1-59259-374-7:115.
[17]B. Shrum et al., “A robust scoring system to evaluate sepsis severity in an animal model,” BMC Research Notes, vol. 7, no. 1, p. 233, Apr. 2014, doi: 10.1186/1756-0500-7-233.
[18]G. Matute-Bello, C. W. Frevert, and T. R. Martin, “Animal models of acute lung injury,” American Journal of Physiology-Lung Cellular and Molecular Physiology, vol. 295, no. 3, pp. L379-L399, Sep. 2008, doi: 10.1152/ajplung.00010.2008.
[19]C. W. McIlwraith, D. D. Frisbie, and C. E. Kawcak, “The OARSI histopathology initiative: recommendations for histological assessments of osteoarthritis in the horse,” Osteoarthritis Cartilage, vol. 18, no. Suppl 3, 2010.
[20]G. Hannum et al., “Genome-wide Methylation Profiles Reveal Quantitative Views of Human Aging Rates,” Mol Cell, vol. 49, no. 2, pp. 359-367, 2013, doi: 10.1016/j.molcel.2012.10.016.
[21]S. Horvath and K. Raj, “DNA methylation-based biomarkers and the epigenetic clock theory of ageing,” Nat Rev Genet, vol. 19, no. 6, pp. 371-384, Jun. 2018, doi: 10.1038/s41576-018-0004-3.
[22]R. Dhingra et al., “Evaluating DNA methylation age on the Illumina MethylationEPIC Bead Chip,” PLoS One, vol. 14, no. 4, p. e0207834, Apr. 2019, doi: 10.1371/journal.pone.0207834.
[23] Weidner, C I, “Aging of blood can be tracked by DNA methylation changes at just three CpG sites,” Genome Biology, vol. 15, no. R24, Feb. 2014, doi: https://doi.org/10.1186/gb-2014-15-2-r24.
[24]G. Biziulevičienė, G. Puidokaitė, A. Šiaurys, and M. Mauricas, “An anti-inflammatory effect of murine fetal liver cells in BALB/c mouse contact hypersensitivity model,” International Immunopharmacology, vol. 7, no. 6, pp. 744-749, Jun. 2007, doi: 10.1016/j.intimp.2007.01.012.

What is claimed is:

1. A method for preparing EVs from Muse cells comprising the steps of:
(a) providing Muse cells;
(b) culturing the Muse cells of (a) in a first medium for a time period sufficient to expand Muse cells in an undifferentiated state;
(c) culturing the Muse cells of (b) in a second medium for a time period sufficient to induce production of EVs, wherein the EVs are released into the second medium; and
(d) harvesting the EVs for one or more times, wherein each harvesting comprises collecting the second medium comprising the EVs to obtain a harvest.

2. The method of claim 1, wherein:
(i) the undifferentiated Muse cells of (b) express SSEA-3, optionally wherein the undifferentiated Muse cells of (b) further express one or more of CD105, CD90, and CD73.
(ii) the Muse cells of (a) are provided as a single cell suspension; and/or
(iii) the culturing of Muse cells of (b) comprises expanding the Muse cells by a factor of at least 2, 5, 10, 20, 30, 40, 50 or 60.

3. The method of any one of claim 1 or 2, wherein:
(i) each harvesting of step (d) comprises replacing the medium comprising the EVs with a fresh second medium;
(ii) the harvesting (d) is repeated once, twice, three times or more; and/or
(iii) after harvesting (d) the Muse cells are re-plated at sub-confluent density, and repeating steps (b)-(d).

4. The method of any one of claims 1 to 3, further comprising a step of:
(e) processing of the harvest obtained in (d); and optionally
(f) subjecting the processed harvest of step (e) to one or more purification steps, wherein the purification steps optionally comprise ultrafiltration.

5. The method of any one of claims 1 to 4, wherein the Muse cells are obtained from umbilical cord, cord blood, placenta or Wharton's jelly.

6. A plurality of extracellular vesicles (EVs) obtainable by the method of any one of claims 1 to 5.

7. The plurality of EVs of claim 6, wherein the EVs have a mean diameter of about 1000 nm or less, optionally of less than 1000 nm, 900 nm, 800 nm, 700 nm, 600 nm, 500 nm, 400 nm, 300 nm or 200 nm.

8. A pharmaceutical composition comprising a pharmaceutically effective amount of a plurality of EVs of any one of claim 6 or 7 and a pharmaceutically acceptable carrier.

9. The plurality of EVs of any one of claim 6 or 7 or the pharmaceutical composition of claim 8 for use in therapy.

10. The plurality of EVs of any one of claim 6 or 7 or the pharmaceutical composition of claim 8 for use in a method of treating inflammation or an inflammatory disorder in a subject in need thereof, said method comprising administering a therapeutically effective amount of said composition or said plurality of EVs to said subject.

11. The plurality of EVs or the pharmaceutical composition for use of any of claim 9 or 10, said method comprising administration of at least one additional anti-inflammatory agent, optionally wherein said anti-inflammatory agent is an anti-inflammatory miRNA.

12. The plurality of EVs or the pharmaceutical composition for use of any of claims 9 to 11, wherein said miRNAs comprise one or more of miR-199a-3p, miR-143-3p, miR-21-5p, miR-125b-5p, let-7b-5p, miR-29a-3p, let-7i-5p, miR-125a-5p, miR-16-5p, miR-221-3p, miR-432-5p, miR-127-3p, miR-382-5p, miR-146a-5p, miR-431-5p, let-7a-5p, miR-199b-3p, miR-100-5p, miR-6504-3p, miR-26a-5p, let-7a-5p, let-7f-5p, miR-26a-5p, miR-431-5p, miR-126-3p, miR-181a-5p, let-7e-5p.

13. Non-therapeutic use of the plurality of EVs of any one of claim 6 or 7 or the pharmaceutical composition of claim 8 for:
(i) preventing hair loss or promoting hair growth in a subject; and/or
(ii) improving the appearance of wrinkles of the skin of a subject.

14. A plurality of extracellular vesicles (EVs) obtained from Muse cells or a tissue comprising Muse cells.

15. The plurality of EVs according to claim 14, wherein the Muse cells are obtained from placenta, umbilical cord, cord blood and/or Wharton's jelly.

16. A method for enriching Muse cells in culture comprising the steps of:
(a) providing a mixture of cells comprising Muse cells from a tissue in a seeding medium;
(b) culturing the cells of (a) in a expansion medium for a time period sufficient to expand Muse cells in an undifferentiated state,
wherein Muse cells after step (b) are enriched at least by a factor of 2, 3, 4, 5, 10, 100, 10³, 10⁴, or 10⁵ relative to the amount of Muse cells present in step (a).

17. Method of claim 16, wherein the seeding medium of (a) comprises one or more of human cord blood plasma, human platelet lysate, insulin, and/or transferrin at a concentration of 1-25%, and/or
wherein the expansion medium of (b) comprises one or more of human cord blood plasma, human platelet lysate, fibroblast growth factor, stem cell factor, VEGF, transferrin, selenium, insulin, human growth hormone, nerve growth factor, vitamin C, D, E, A, and valproic acid.

18. Method of any one of claim 16 or 17, wherein the cells of (a) are obtained from mesenchymal stem cells, adipose-derived stem cells, bone-marrow-derived stem cells, placenta, umbilical cord, cord blood, Wharton's jelly, cartilage, adipose tissue, bone marrow or dermal fibroblastic tissue.

19. Method of any one of claims 16 to 18, wherein the culture of step (b) comprises at least 10%, at least 20%, at least 40%, at least 50%, at least 60%, at least 70%, or at least 80 Muse cells after enrichment.

20. Composition comprising at least 10% Muse cells obtainable by the method of any one of claims 16 to 19
